SEQUENCE LISTING (40 sequences)

SEQ ID NO: 1 - DNA, 417 bp, Mus musculus
atgtcctggagacgggtcattctcctgtcatctctcttggccctggtgctcctgtgtatgctacaggaggggaccagcgcttctgtggggagcaggcaggcagctgcagagggggtgcaggaaggtgtgaaacagaagattttcatgcaagaatctgatgcctccaatttcctcaagaggcgtggcaagcggtctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatctga

SEQ ID NO: 2 - DNA, 351 bp, Mus musculus
atgtcctggagacgggtcattctcctgtcatctctcttggccctggtgctcctgtgtagtgtgaaacagaagattttcatgcaagaatctgatgcctccaatttcctcaagaggcgtggcaagcggtctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatctga

SEQ ID NO: 3 - protein, 138 aa, Mus musculus
MSWRRVILLSSLLALVLLCMLQEGTSASVGSRQAAAEGVQEGVKQKIFMQESDASNFLKRRGKRSPKSRDEVNAENRQRLRDDELRREYYEEQRNEFENFVEEQRDEQEERTREAVEQWRQWHYDGLYPSYLYNRQNI

SEQ ID NO: 4 - protein, 138 aa, Rattus norvegicus
MSWRQVILLSSLSALVLLCMLQEGTSASVGSRQAAGEEVQEGMKQKIFMQESDASNFLKRRGKRSPKSRDEVTAENRQKLRDDELRREYYEEQRNEFENFVEEQRDEQEERTREAVEQWRQWHYDGLYPSYLYNRQNI

SEQ ID NO: 5 - protein, 138 aa, Homo sapiens
MTWRQAVLLSCFSAVVLLSMLREGTSVSVGTMQMAGEEASEDAKQKIFMQESDASNFLKRRGKRSPKSRDEVNVENRQKLRVDELRREYYEEQRNEFENFVEEQNDEQEERSREAVEQWRQWHYDGLHPSYLYNRHHT

SEQ ID NO: 6 - DNA, 21 bp, Artificial Sequence
  Description of Artificial Sequence Synthetic primer
atgtcctggagacgggtcatt

SEQ ID NO: 7 - DNA, 21 bp, Artificial Sequence
  Description of Artificial Sequence Synthetic primer
tacaaccgccaaaacatctga

SEQ ID NO: 8 - DNA, 23 bp, Artificial Sequence
  Description of Artificial Sequence Synthetic primer
cctgtcatctctcttggccctgg

SEQ ID NO: 9 - DNA, 21 bp, Artificial Sequence
  Description of Artificial Sequence Synthetic primer
acgaggagcaaaggaacgagt

SEQ ID NO: 10 - DNA, 22 bp, Artificial Sequence
  Description of Artificial Sequence Synthetic primer
gtcccgagatgaagttaatgcg

SEQ ID NO: 11 - DNA, 21 bp, Artificial Sequence
  Description of Artificial Sequence Synthetic primer
acgaggagcaaaggaacgagt

SEQ ID NO: 12 - DNA, 1630 bp, Mus musculus
ggcggactagtggccttggaagctgcaagggacccgcgcactgcggccagagaacagcgacctgccttccccagcagcaaggctgtgagtggcaagatgggaccgggctcacagcgcgcgcttttcctcctcttgctgctcctcgccagccccggagcgcgggctttccagagctgtctcaacaaacagcagctcctcaccaccatccgccagctgcagcagctgctgaaaggccaggagacccgcttcaccgaagcgatccgaaacatgaagagccggctggctgcactgcaaaacacggttaacaaaatgaccccggatgcaccaccagtttcctgcccagctctggaggcccctccggatggcaagaagtttggaagcaagtacttagtggaccatgaagtctattttacctgcaaccctgggttccagctggttgggcccagcagcgtggtgtgtcttgctaatggtagctggacaggagagcagccccgctgcagagatatcagtgaatgctccagccagccttgtcacaatggagggacgtgtgtggaaggcatcaaccactacagatgcatctgtcctccaggaaaaactgggaatggctgtcagcatcagacccaggctgcggccccagatggcggcgaggcggtgacccgccttcagccgcgcacccgctgcgcgcaggtggagcgggaacagcattgcagctgcgaggcgggattccacctgagcagcaccacgggcggccacagcgtctgccaggatgtgaatgagtgtgagatctatgggcagaaaggacgcccccggctctgcatgcatgcctgtgtgaacacccctggttcctaccgatgtacctgcccgagtggataccggatcctggctgatgggaagagctgtgaggatgttgatgagtgtgcaggcccacaacacatgtgcccccgggggaccacatgcatcaacactggaggaggcttccagtgtgtcaaccctgagtgtcctgagggcagcggcaatataagctacgtgaagacatctccctttcagtgcgagcgaaacccttgtcccatggacagcaggccatgccgccacctgcccaagaccatctccttccattacctctctctcccttccaagttgaagacacccatcacgctcttccgcatggccacagcctcaattcctggccatcctgggcccaacagcctgcgctttgggatcgtgggtgggaacagccgtggccacttcgtaatgcagcgctcagaccggcagacaggagagctcatccttacacagaccctggaggggcctcagactctggaggttgacgtagacatgtcggaatacctggagcgctccttccaggccaaccatgtatccaaggtcaccatctttgtttctcgctatgacttctgaggatgccatggcagtcgggaggctggggtttgagatctgggctgatttcacttccccaaggacacattgtgggcaagagctgtggtggtcattcttttcttggtcatgttcttacctactctgcttgttcattcaggcttctagatcaatgctgtgttctgacccaggggctgccacagaagggaagccacaaacaaatgctgtatctaccatc

SEQ ID NO: 13 - protein, 567 aa, Rattus norvegicus
MTTGPALSSSEEGLMSEMPPAWCHTARADHSTPGTSPEMIPDTQGHWMPLRSRGLGALPGAFQAPQPLLSRLRHRLAPPRIVGTVSCRRLRTIVALGAARDSCTVGTQGRPGNSDLPAFPTSQVVRGKMGPGSQRTLVLLLLLLASPGARAFQSCLNKQQLLTTVRQLQQLLKGQETRFTEGIRNMKSRLTALQNTVSKMTPDAPPVSCPALDAPPNGKKFGSKYLVDHEVHFTCNPGFQLVGPSSVVCLANGTWTGEQPRCRDTSECSSQPCHNGGTCVEGVHHYRCICPPGKTGNRCQHQTQAAAPGGVAGDSAYSRAPRCAQVEREQHCSCEAGFHLSGTAGGHSVCQDVNECEIYGQEGRPRLCMHACVNTPGSYRCTCPSGYRILADGKSCEDVDECAGPQHMCPRGTTCINTGGGFQCVNPECPEGSGNISYVKTSPFQCERNPCPMDSRPCRHLPKTISFHYLSLPSNLKTPITLFRMATASVPGHPGPNSLRFGIVGGNSRGHFVMQRSDRQTGELILIQTLEGPQTLEVEVDMSEYLERSFQASHVSKVTIFVSRYDF

SEQ ID NO: 14 - protein, 439 aa, Mus musculus
MGPGSQRALFLLLLLLASPGARAFQSCLNKQQLLTTIRQLQQLLKGQETRFTEAIRNMKSRLAALQNTVNKMTPDAPPVSCPALEAPPDGKKFGSKYLVDHEVYFTCNPGFQLVGPSSVVCLANGSWTGEQPRCRDISECSSQPCHNGGTCVEGINHYRCICPPGKTGNGCQHQTQAAAPDGGEAVTRLQPRTRCAQVEREQHCSCEAGFHLSSTTGGHSVCQDVNECEIYGQKGRPRLCMHACVNTPGSYRCTCPSGYRILADGKSCEDVDECAGPQHMCPRGTTCINTGGGFQCVNPECPEGSGNISYVKTSPFQCERNPCPMDSRPCRHLPKTISFHYLSLPSKLKTPITLFRMATASIPGHPGPNSLRFGIVGGNSRGHFVMQRSDRQTGELILTQTLEGPQTLEVDVDMSEYLERSFQANHVSKVTIFVSRYDF

SEQ ID NO: 15 - protein, 439 aa, Homo sapiens
MVPSSPRALFLLLLILACPEPRASQNCLSKQQLLSAIRQLQQLLKGQETRFAEGIRHMKSRLAALQNSVGRVGPDALPVSCPALNTPADGRKFGSKYLVDHEVHFTCNPGFRLVGPSSMVCLPNGTWTGEQPHCRGISECSSQPCQNGGTCVEGVNQYRCICPPGRTGNRCQHQAQTAAPEGSVAGDSAFSRAPRCAQVERAQHCSCEAGFHLSGAAGDSVCQDVNECELYGQEGRPRLCMHACVNTPGSYRCTCPGGYRTLADGKSCEDVDECVGLQPVCPQGTTCINTGGSFQCVSPECPEGSGNVSYVKTSPFQCERNPCPMDSRPCRHLPKTISFHYLSLPSNLKTPITLFRMATASAPGRAGPNSLRFGIVGGNSRGHFVMQRSDRQTGDLILVQNLEGPQTLEVDVDMSEYLDRSFQANHVSKVTIFVSPYDF

SEQ ID NO: 16 - DNA, 2685 bp, Mus musculus
aagaggtaatcctgcagctgtgccatctgcgttaaactgctgcgtggatcgtgccagttcaccgtggagggagagatgctcatcgagccaaattgatcattgcagccgcagggcagtgacatctgtctctaagtcctccctaggagcgcgacccgcactgtctccttccaggagcccgtcatttcctcgacttttgagaggtgtctctccccagcccgaccgtccagatgcgtttttgcctcttctcatttgccctcatcattctgaactgtatggattacagccagtgccaaggcaaccgatggagacgcaataagcgagctagttatgtatcaaatcccatttgcaagggttgtttgtcttgttcgaaggacaatggttgcagccgatgtcaacagaagttgttctttttccttcgaagagaaggaatgcgtcagtatggagagtgcctgcattcctgcccatcagggtattatggacaccgagccccagatatgaacagatgtgcacgatgcagaatagaaaactgtgattcttgctttagcaaagacttttgtacgaagtgcaaagtaggcttttatttgcatagaggccgctgctttgatgaatgtccagatggttttgcaccgttagatgagactatggaatgtgtagaaggttgtgaagttggtcattggagcgaatgaggaacgtgtagcagaaacaaccgcacgtgtggatttaaatggggtctggaaaccagaacacggcagattgttaaaaagccagcaaaagacacaataccatgtccgaccattgcggagtccaggagatgcaagatggccatgaggcactgtccaggaggaaagagaacaccaaaggcaaaagagaagagaaacaagaagaagaggcggaagctgattgagagagcccaagagcagcacagcgtcttcctcgctacagacagagtgaaccaataaaatacaagaaatagctggggcattttgaggttttctgttttgtttatgttgttgttttgcaaaagtgcacaaagctactctccagtccacactggtggacagcattcctgatcctctgaccagtatccattttcagtaatgctgcagagggaggtgcccaagcatggactcagcgttatttatgctttgattggaatctggggcctgtgatggcaggagcttgttgagctgagtcagcgggagctgatgcatctgtactcttgtgatgagcacagtgtgtcataagaacctgtccctggcacggtggacccacaggaggcacaaggctgtagatcaccaccagagaatgcacctgtgcctattttgatggatggcaatgctaagcaagcaagcactgttcacttgtgactttcatttctcacactgtgcactgtcaaagacaaatgtgcatggaaaaatgtttagtgtcacctcatggcgttctcagcatcagtgaccttcaaacggtcctacaatgagactgtgttctagctaggggtatgctgtggaaattcctgccacatttcatcttagtgctaacatgtacagattctgctgcgctacattcaaagctcattactgtatatttatgctttctctgtgtaacaagttatacctgataagatgtcactttgtttctagtggttcttaaccatggtctggtacatggctattctagttttggaaattaacaagtgttttgttgcctcttgttttcttttgttcctatcatttttggcgggggtgggatgggcttgattctaaccgtaagtataggataagctagttttgtatatagagtcaaatgactgatgtcagaggatcagtgctgatagaacttccccagttcatgtcaccatacgcacagagagaaagcagcatgaggcatcttgccatcagaagccaaatttcttttgagtcccaaaattgatgacttatgaaatatagctgaaaacaagatttgggtgtagttacttgtatttattatacaatttccaattacattttttttcaaactcaaaataacccatgactttgagtgataggtcacttggcaatgttcttgaattactggggaagctgttgtcactaagataatgagagagaaaatagaatggcttcgcccaagtgagagccacatcttacatttctctgttgaatcggaatcaactatattagaacagaagcctgatggaagctttctagttaacacacacaaggccatggtttcaaaaacatctttgtccccttaggtcagtttgtccttagattatgaattggcaggttctaattgcattatttccctggctgatccaggaaaaagttagaacaaaataagttgcatagttttgaggaaacatccaaagcaaggcgaagcctttccttgccttgcattggcaaaactacctctttagcatttatgttgattcagaaacatcttgctgatatgtgtagatgttttaagcttcattgtgaaaatattgatgcaagataagccatatatgaatgttgtattcaactttagggcttgaaattaatcctaaagtgttcacctctctccatgtctatttacactctgttcctatttactaagagggtaggggtctccttaatatcatacttcattgttaataagtcaatgcttgttatgtttcttggctgttgtttttgtgctaaaaaaaaaaaa

SEQ ID NO: 17 - DNA, 930 bp, Mus musculus
ggtctaaggaacatggccccggggccgtcagccacacaggggatcctgctgctgctgcctctcctgcctctgtcccaggtgacgctgggttccgcggaccgcaactgtgacccctcggatcagtgcccgccccaggcccgctggagcagcctatggcatgtggggctcatcctgctcgccatactcctgatgcttttgtgtggggtcacagccagctgtgtacgattctgctgcctcaggaagcagacgcacacccagtcacacacgccagcggcgtggcagccctgtgacgggacagtcatcccagtggacagtgatagccccgcacacagcactgtgacctcctacagttccgtgcagtacccactgggcatgcggctgcccctgtactttggagagccagaccctgactccatggtccctcccacctacagcctctatgcgtctgagctgccaccctcctacgatgaagttgtgaagatgataaaagccagggaggaagtggcagctccctctgagaaaaccaactctctgcctgaggccttggagccagagaccactggagggccccaggagccaggccccagtgcccagcggccctagtcaagcccgcttgaaaagggtgggagttctctcttctggagccattaaaccattgcctggattcctggcacttaggaccaaggagccccacctgtgtcatccttcccctgggaaggccagccatccagcacattgctgcggacgcggcctcgttgctatgcaatgcctggccagccagcctgcccctgtattggcccctcttcttcacccttcccatccatcgggggcccagcaggccctcatcccggaggagcctggatcttgggagctcaaaccccactggcatgccagccctttgaggggaaaataaagtttacgtaaacatgt

SEQ ID NO: 18 - DNA, 3071 bp, Mus musculus
aggcgatcctgcatgctttggagctcctgttgatcagaaactacagccccaaaagatctttcttcattgctttgggccatgatgaggaggtgtccggggaaaagggggctcagaagatctcagcactcttacaggcaaggggtgtccagctagccttccttgtggatgaagggagctttatcttggaaggcttcattccaaacctcgagaagccagttgccatgatttcagtcactgagaagggtgcccttgacctcatgctgcaagtaaacatgactccaggccactcttcagctcccccaaaggagacaagcattggcattctttctgccgctgtcagccgactggagcagacaccaatgccgaatatgtttggaggagggccattgaagaagacaatgaagctactggcaaatgagttttccttccctatcaatatagtcttgagaaacctgtggctatttcatcccattgtgagcaggataatggagaggaaccccataacaaatgcgctggtccgaactaccacagccctcaccatgttcaatgcaggaatcaaggtgaatgtcatccctccattggctcaggctacaatcaactgccgaattcacccttcgcagacagtacatgaggtcctagaacttgtcaagaacaccgtggctgatgacagagtccagctgcatgtgttgagatcctttgaacccctgcccatcagcccctctgatgaccaggccatgggctaccagctgcttcaagagaccatacgatctgtcttcccggaagtcgacatcgtggtccccggtatttgtattgccaatacggacacccgacactatgccaacatcaccaatggcatgtaccggttcaacccccttcccctgaaccctcaggacttcagtggtgtccatggaatcaatgagaaagtttccgttcagaactaccagaaccaggtgaagttcatctttgagttcatccaaaatgccgacacttacaaagagccagttcctcatctgcatgaactatgaggcaaggatccagctgggtgagggatgcccagcactgggctcaggactaacctaaagggagagagagctggtattaatgaagggtttggtggaaaccatcctgaacagagttcacctgcctgacttccctcctccactcaccagggattgaggtcccaggaggaaagggagatgaacagaatctgccctgctgtcctcctgacatctgcgttcttcctctgagtggcaactactgccttctggtgtgtcctgcctgttggtctgtctcacgccgattattaactattccactttcccagcctgtcttaacaaatacagattttggaaagacctcaagcaggtttaaatactagagtattgacttcaggtacctgaggctggactgtcttctagaatgccctttctcctgtcttctctccgactcgggagctgccccttggcctctctcttcctgcctctgcttccttcctccagtctccagctcattccacatctggagttaagactcaaacttagaagctcccatgatcacagtcaaatagaagggcctgtgtaatgaaaatgtcagctggtctctgacaggtgagcttgaggtggagactttgagagaaccccagaactcctggttccagagacttgggctcttgggactttgttcccctgccctgccctgttcctgccacctgctgtccggccccaccccaggaagcattactctacagaagcatttccctggcacagtgtacgggctgagctcccaagagaggaacactcaaaatccagcttctcagcggtgcccagagccagcagcgccctggtcctcgtgtgtcccatttcagaatgaccaaggggaagtggaggacaaggaggacaagagaagcaaagggaggtagcgtccattaaagtatagcagagctcctgtcttcgctctggacgtcaaccctaggctatttgaatgtgcccagtagctgccaggtggcgggcactgggaagcagcctttcttggctcaagaccagcatctctgctctgtggctcacaagggagtggaggtcacaaaggaagacacagaaatggagcttggaaggctccgtctcttcccctctccagactaggaccttgtgtgtctcccaaggctttgacagttctaggtttgcttgcctcctcctcagagatctccattctccctcactcccaagtccttaccttccccccaggatatgaacggttccttgaagctctggcttaatgggtaacgtttagaaactgtggggattattccagctttttagctactcctgctaagattcacattccttccccgactctccctttttggacaattcagtacttattcagccttcattgaagggcaagcttaagaacaaaaatagtccttcacttttgtgcagagccctacgagatagtcttatagacgcttcatctcagccctgtgaacacgctatcattactccttctttcagagatgggctcgattctggaaacagtgttcaaaccttcattactttgttcccagcctcatcagtattctctgatcctgcatcacctactccccatcctacatcacctactccccactgatttcttctttaattctcatgactactgaggcatgagtgtgtaatcacatgagggaagtattcacagggagggaacatagccggctggggcttgcttttgtagggttattaggattagacaaggaatggggctgagctccattaattaatggctttttaaggagagaatttgagaaaatagacacatttaatctgatttggaaaatgtaccatgaatacatgtgtcaaaatatcacagtacttcactaaattaaggggaaaagagaacagaagacagacgggttcatacatatattccctgtgtcttgccatgtgaagtcctataccatctcttatgcgaggaaacctattcatcagggggcaaactagtgttccccacatgattttaatgctccagagctgtgagccaaaataaaccatgctcaacccggaaaaaaaaaaaaaaaa

SEQ ID NO: 19 - DNA, 940 bp, Homo sapiens
ctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggagaaaaaaaaaaaaaaaaaaaaaaaaaaaaaaaaaaaaaaaaaaaaaaaaaaaaaaa

SEQ ID NO: 20 - DNA, 711 bp, Homo sapiens
gcctggggtcacagggatgccgcggctcctccgcttgtccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcaaaatttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacatttatttaaaaa

SEQ ID NO: 21 - protein, 213 aa, Homo sapiens
MPRLLRLSLLWLGLLLVRFSRELSDISSARKLCGRYLVKEIEKLCGHANWSQFRFEEETPFSRLIAQASEKVEAYSPYQFESPQTASPARGRGTNPVSTSWEEAVNSWEMQSLPEYKDKKGYSPLGKTREFSSSHNINVYIHENAKFQKKRRNKIKTLSNLFWGHHPQRKRRGYSEKCCLTGCTKEELSIACLPYIDFKRLKEKRSSLVTKIY

SEQ ID NO: 22 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 23 - protein, 480 aa, Homo sapiens [sequence omitted]

SEQ ID NO: 24 - DNA, 1715 bp, Homo sapiens
gaattccagcggcggcgccgttgccgctgccgggaaacacaaggaaagggaaccagcgcagcgtggcgatgggcgggggtagagccccgccggagaggctgggcggctgccggtgacagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcgaaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttacccctgcc

SEQ ID NO: 25 - DNA, 1703 bp, Homo sapiens
gcagcagcagagaatccaaaccctaaagctgatatcacaaagtaccatttctccaagttgggggctcagaggggagtcatcatgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatgtcagcaatcagattgtggcatgctgggtaactggaaaaaataataaaaatcggcttcctacagccagcagcacagtcacccatggaactgttggctttggattaaatgtggaattgaacgactacccagaagtgttctggaaagaagcgagatgtgtggcctgcctcaccgtcctcacccatcaaaagcaccagcaggcacgttaactcgaattctcacaaggaaaaggccattaaagctcaaggtgcatttcaaactccaggctac

SEQ ID NO: 26 - DNA, 5061 bp, Homo sapiens
atggctcagatatttagcaacagcggatttaaagaatgtccattttcacatccggaaccaacaagagcaaaagatgtggacaaagaagaagcattacagatggaagcagaggctttagcaaaactgcaaaaggatagacaagtgactgacaatcagagaggctttgagttgtcaagcagcaccagaaaaaaagcacaggtttataacaagcaggattatgatctcatggtgtttcctgaatcagattcccaaaaaagagcattagatattgatgtagaaaagctcacccaagctgaacttgagaaactattgctggatgacagtttcgagactaaaaaaacacctgtattaccagttactcctattctgagcccttccttttcagcacagctctattttagacctactattcagagaggacagtggccacctggattacctgggccttccacttatgctttaccttctatttatccttctacttacagtaaacaggctgcattccaaaatggcttcaatccaagaatgcccacttttccatctacagaacctatatatttaagtcttccgggacaatctccatatttctcatatcctttgacacctgccacaccctttcatccacaaggaagcttacctatctatcgtccagtagtcagtactgacatggcaaaactatttgacaaaatagctagtacatcagaatttttaaaaaatgggaaagcaaggactgatttggagataacagattcaaaagtcagcaatctacaggtatctccaaagtctgaggatatcagtaaatttgactggttagacttggatcctctaagtaagcctaaggtggataatgtggaggtattagaccatgaggaagagaaaaatgtttcaagtttgctagcaaaggatccttgggatgctgttcttcttgaagagagatcgacagcaaattgtcatcttgaaagaaaggtgaatggaaaatccctttctgtggcaactgttacaagaagccagtctttaaatattcgaacaactcagcttgcaaaagcccagggccatatatctcagaaagacccaaatgggaccagtagtttgccaactggaagttctcttcttcaagaagttgaagtacagaatgaggagatggcagctttttgtcgatccattacaaaattgaagaccaaatttccatataccaatcaccgcacaaacccaggctatttgttaagtccagtcacagcgcaaagaaacatatgcggagaaaatgctagtgtgaaggtctccattgacattgaaggatttcagctaccagttacttttacgtgtgatgtgagttctactgtagaaatcattataatgcaagccctttgctgggtacatgatgacttgaatcaagtagatgttggcagctatgttctaaaagtttgtggtcaagaggaagtgctgcagaataatcattgccttggaagtcatgagcatattcaaaactgtcgaaaatgggacacagaaattagactacaactcttgaccttcagtgcaatgtgtcaaaatctggcccgaacagcagaagatgatgaaacacccgtggatttaaacaaacacctgtatcaaatagaaaaaccttgcaaagaagccatgacgagacaccctgttgaagaactcttagattcttatcacaaccaagtagaactggctcttcaaattgaaaaccaacaccgagcagtagatcaagtaattaaagctgtaagaaaaatctgtagtgctttagatggtgtcgagactcttgccattacagaatcagtaaagaagctaaagagagcagttaatcttccaaggagtaaaactgctgatgtgacttctttgtttggaggagaagacactagcaggagttcaactaggggctcacttaatcctgaaaatcctgttcaagtaagcataaaccaattaactgcagcaatttatgatcttctcagactccatgcaaattctggtaggagtcctacagactgtgcccaaagtagcaagagtgtcaaggaagcatggactacaacagagcagctccagtttactatttttgctgctcatggaatttcaagtaattgggtatcaaattatgaaaaatactacttgatatgttcactgtctcacaatggaaaggatctttttaaacctattcaatcaaagaaggttggcacttacaagaatttcttctatcttattaaatgggatgaactaatcatttttcctatccagatatcacaattgccattagaatcagttcttcaccttactctttttggaattttaaatcagagcagtggaagttcccctgattctaataagcagagaaagggaccagaagctttgggcaaagtttctttacctctttgtgactttagacggtttttaacatgtggaactaaacttctatatctttggacttcatcacatacaaattctgttcctggaacagttaccaaaaaaggatatgtcatggaaagaatagtgctacaggttgattttccttctcctgcatttgatattatttatacaactcctcaagttgacagaagcattatacagcaacataacttagaaacactagagaatgatataaaagggaaacttcttgatattcttcataaagactcatcacttggactttctaaagaagataaagcttttttatgggagaaacgttattattgcttcaaacacccaaattgtcttcctaaaatattagcaagcgccccaaactggaaatggggtaatcttgccaaaacttactcattgcttcaccagtggcctgcattgtacccactaattgcattggaacttcttgattcaaaatttgctgatcaggaagtaagatccctagctgtgacctggattgaggccattagtgatgatgagctaacagatcttcttccacagtttgtacaagctttgaaatatgaaatttacttgaatagttcattagtgcaattccttttgtccagggcattgggaaatatccagatagcacacaatttatattggcttctcaaagatgccctgcatgatgtacagtttagtacccgatacgaacatgttttgggtgctctcctgtcagtaggaggaaaacgacttagagaagaacttctaaaacagacgaaacttgtacagcttttaggaggagtagcagaaaaagtaaggcaggctagtggatcagccagacaggttgttctccaaagaagtatggaacgagtacagtccttttttcagaaaaataaatgccgtctccctctcaagccaagtctagtggcaaaagaattaaatattaagtcgtgttccttcttcagttctaatgctgtccccctaaaagtcacaatggtgaatgctgaccctctgggagaagaaattaatgtcatgtttaaggttggtgaagatcttcggcaagatatgttagctttacagatgataaagattatggataagatctggcttaaagaaggactagatctgaggatggtaattttcaaatgtctctcaactggcagagatcgaggcatggtggagctggttcctgcttccgataccctcaggaaaatccaagtggaatatggtgtgacaggatcctttaaagataaaccacttgcagagtggctaaggaaatacaatccctctgaagaagaatatgaaaaggcttcagagaactttatctattcctgtgctggatgctgtgtagccacctatgttttaggcatctgtgatcgacacaatgacaatataatgcttcgaagcacgggacacatgtttcacattgactttggaaagtttttgggacatgcacagatgtttggcagcttcaaaagggatcgggctccttttgtgctgacctctgatatggcatatgtcattaatgggggtgaaaagcccaccattcgttttcagttgtttgtggacctctgctgtcaggcctacaacttgataagaaagcagacaaacctttttcttaacctcctttcactgatgattccttcagggttaccagaacttacaagtattcaagatttgaaatacgttagagatgcacttcaaccccaaactacagacgcagaagctacaattttctttactaggcttattgaatcaagtttgggaagcattgccacaaagtttaacttcttcattcacaaccttgctcagcttcgtttttctggtcttccttctaatgatgagcccatcctttcattttcacctaaaacatactcctttagacaagatggtcgaatcaaggaagtctctgtttttacatatcataagaaatacaacccagataaacattatatttatgtagtccgaattttgtgggaaggacagattgaaccatcatttgtcttccgaacatttgtcgaatttcaggaacttcacaataagctcagtattatttttccactttggaagttaccaggctttcctaataggatggttctaggaagaacacacataaaagatgtagcagccaaaaggaaaattgagttaaacagttacttacagagtttgatgaatgcttcaacggatgtagcagagtgtgatcttgtttgtactttcttccaccctttacttcgtgatgagaaagctgaagggatagctaggtctgcagatgcaggttccttcagtcctactccaggccaaataggaggagctgtgaaattatccatctcttaccgaaatggtactcttttcatcatggtgatgcatatcaaagatcttgttactgaagatggagctgacccaaatccatatgtcaaaacatacctacttccagataaccacaaaacatccaaacgtaaaaccaaaatttcacgaaaaacgaggaatccgacattcaatgaaatgcttgtatacagtggatatagcaaagaaaccctaagacagcgagaacttcaactaagtgtactcagtgcagaatctctgcgggagaattttttcttgggtggagtaaccctgcctttgaaagatttcaacttgagcaaagagacggttaaatggtatcagctgactgcggcaacatacttgtaa

SEQ ID NO: 27 - DNA, 2823 bp, Homo sapiens
agattcactggtgtggcaagttgtctctcagactgtacatgcattaaaattttgcttggcattactcaaaagcaaaagaaaagtaaaaggaagaaacaagaacaagaaaaaagattatattgattttaaaatcatgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcatgagatttatattaagcgttcataacttcctaaaacatggaaggttttcccctcaacaattttgaagctgtgaaattaagtaccacaggctataggcctagagtatgctacagtcacttaagcataagctacagtatgtaaactaaaagggggaatatatgcaatggttggcatttaaccatccaaacaaatcatacaagaaagttttatgatttccagagtttttgagctagaaggagatcaaattacatttatgttcctatatattacaacatcggcgaggaaatgaaagcgattctccttgagttctgatgaattaaaggagtatgctttaaagtctatttctttaaagttttgtttaatatttacagaaaaatccacatacagtattggtaaaatgcaggattgttatataccatcattcgaatcatccttaaacacttgaatttatattgtatggtagtatacttggtaagataaaattccacaaaaatagggatggtgcagcatatgcaatttccattcctattataattgacacagtacattaacaatccatgccaacggtgctaatacgataggctgaatgtctgaggctaccaggtttatcacataaaaaacattcagtaaaatagtaagtttctcttttcttcaggggcattttcctacacctccaaatgaggaatggattttctttaatgtaagaagaatcatttttctagaggttggctttcaattctgtagcatacttggagaaactgcattatcttaaaaggcagtcaaatggtgtttgtttttatcaaaatgtcaaaataacatacttggagaagtatgtaattttgtctttggaaaattacaacactgcctttgcaacactgcagtttttatggtaaaataatagaaatgatcgactctatcaatattgtataaaaagactgaaacaatgcatttatataatatgtatacaatattgttttgtaaataagtgtctccttttttatttactttggtatatttttacactaaggacatttcaaattaagtactaaggcacaaagacatgtcatgcatcacagaaaagcaactacttatatttcagagcaaattagcagattaaatagtggtcttaaaactccatatgttaatgattagatggttatattacaatcattttatatttttttacatgattaacattcacttatggattcatgatggctgtataaagtgaatttgaaatttcaatggtttactgtcattgtgtttaaatctcaacgttccattattttaatacttgcaaaaacattactaagtataccaaaataattgactctattatctgaaatgaagaataaactgatgctatctcaacaataactgttacttttattttataatttgataatgaatatatttctgcatttatttacttctgttttgtaaattgggattttgttaatcaaatttattgtactatgactaaatgaaattatttcttacatctaatttgtagaaacagtataagttatattaaagtgttttcacatttttttgaaagacaaaaa

SEQ ID NO: 28 - DNA, 3010 bp, Mus musculus
cacgcgtccgcccacgcgtccgctgaaaggagactgtaaaaagtggctctggtgtgtggagctgtggccagggcccctaccactcagctgggagacccaagacgatactgtatccgaggagcctcctgcatgtcctgctgccctgagctcactcaagctaggtgacagcgtgtgaatgctgccaccatgaatgaggtatctgtcatcaaagaaggctggctccacaaacgtggtgaatacatcaagacctggaggccacggtacttccttctgaagagtgatggatctttcattgggtataaggagaggcccgaggcccctgaccagaccttaccccccctgaacaatttctctgtagcagaatgccagctgatgaagactgagaggccacgacccaacacctttgtcatacgctgcctgcagtggaccacagtcatcgagaggaccttccatgtagactctccagatgagagggaagagtggatgcgggctatccagatggtcgccaacagtctgaagcagcggggcccaggtgaggacgccatggattacaagtgtggctcccccagtgactcttccacatctgagatgatggaggtagctgtcaacaaggcacgggccaaagtgaccatgaatgacttcgattatctcaaactcctcggcaagggcaccttcggcaaggtcattctggttcgagagaaggccactggccgctattatgccatgaagatcctgcgcaaggaggtcatcattgcaaaggatgaagtcgcccacacagtcacagagagccgggttctgcagaataccaggcaccccttccttacagccctcaagtatgccttccagacccatgaccgcctatgctttgtgatggagtatgccaacgggggtgagctgtttttccacctctctcgggagcgagtcttcacggaggatcgggcgcgcttttatggagcagagattgtgtcagctctggagtatttgcactcgagagatgtggtgtaccgtgacatcaagctggaaaaccttatgttggacaaagatggccacatcaagatcactgactttggcttgtgcaaagagggcatcagtgatggagccaccatgaaaaccttctgtggtaccccggagtacttggcgcctgaggtgctagaggacaatgactatgggcgagcagtggactggtgggggctgggtgtggtcatgtatgagatgatgtgtggccgcctgccattctacaaccaggaccacgagcgcctctttgagctcattcttatggaggagatccgcttcccgcgcacactcgggccagaggccaagtccctgctggctggactgctgaagaaggacccaaagcagaggctcggcggaggtcccagtgatgcgaaggaggtcatggagcatagattcttcctcagcatcaactggcaggacgtggtacagaaaaagctcctgccacccttcaaacctcaggtcacttcagaagtggacacaaggtactttgatgacgagttcaccgcccagtccatcacaatcacacccccagaccgatatgacagcctggacccgctggaactggaccagcggacgcacttcccccagttctcctactcagccagcatccgagagtgagcagccctctgccaccacaggacacaagcatggccgtcatccactgcctgggtggctttttaaaaaaaaaactttattttgccttttggtttgtgttcccttccctgtcccctcatcccaaatccagtttcttcttcagtccttgctcagacttagtggtcctggcggccctctcctctgggctcccctctgttccaggtgagcgctggggaccgcccagggacaggctgttgggaggatggagagtgaggtcattcctcaccactagcctccaagagggtgaagtggggagccatggggtcacatggatttggaggcacgccagcggaggacacatctagaagttcccctcactcccagcacctcagctgccttccatgctgtcctctgggcaatcttatctgctgccaccccacctggggcacaaaagcagtaatgtccagggacaggcagaagcctttggaagctgaggggtctgggtttgcttctatggagaatggatcttcaagggggatccaggagcaggagctgccttcctcttcctcccaagctactctcttgattctcaataaagcaggaggaatgttcgggggcagccctcctgcccacagcccagtgccttaccagggcttccttctagccagccttgcctcccgctcggcccctttggatggctccgttctctgcctggggcagagggaagccaaccttggctgttacacatctcacaccaagacagtattgggccccttggacttgactggggtctgaggggacagagatggggataggtgtctgatacacaatggggtggagtctgcctcatctccagggcacctgttctggagccttaaagtgaatgtttgagttagagttgtgtcagtgctgtgggtgcgggtgtgcacctgacctagcagtgagcatcccaggctcctgggatgctggtgcccacagctgtatgcagcacttgtgtgagtgggctcaggctcagccccagcctgagtgtttgatcccacggggtgcagtgagtgcaggcgatgtgcctgctgctctggccatgtctctccctcctcaatagctctgggtttttggaaatgagttctcctggttttgctctcccacacacccccagaataggtggatttcagaaaggcagtagggtggggtggttgggatggttatgggggtcatctgttttttactttgtatgtgtgtatgtttatctgagagttttcgcccatccctgctggcctttccttactcctcgtatttgtacggtacaagcaataaagacactcatttcagacccaggaaaaaaaaaaaaaaaaaaaaaaaaaaaaaaaaaaaaaaaaaaaaaaaaaaaaaaaaaaaaaaaaaa

SEQ ID NO: 29 - DNA, 4751 bp, Mus musculus
gcaaagcaccatttctccaagttggggggctcagaggggagccatcatgagcgatgttaccattgtgaaagaaggttgggttcagaagaggggagaatatataaaaaactggaggccaagatacttccttttgaagacagatggctcattcataggctataaggagaaacctcaagatgtggacttaccttatcccctcaacaacttctcagtggcaaaatgtcagttaatgaaaacagaacgaccaaagccaaatacatttattatcagatgtcttcagtggaccactgttatagagagaacatttcatgtagatacaccagaggaaagagaagagtggacggaagctatccaagccgtagccgaccgattgcagaggcaagaggaggagaggatgaattgtagcccagcctcacagattgataatataggagaagaagagatggatgcgtctacaacccatcataaaagaaagacgatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactcttactgaaagcagagtactaaagaacaccagacatccatttttaacatccttgaaatattccttccagacaaaagaccgtttgtgttttgtgatggaatatgttaatggcggagagctgtttttccatttgtcgagagagcgagtgttctctgaggaccgcacacgtttctatggtgcagaaattgtctctgctttggactatctacattctggaaagattgtgtaccgtgatctcaagttggagaatttgatgctagataaggatggccatataaaaattacggattttgggctttgcaaagaagggatcacagatgcagctaccatgaagacattctgtggcacaccagagtacctggcaccagaggtattagaagataatgactatggccgagccgtggactggtggggcttaggtgttgtcatgtatgaaatgatgtgtggaaggttgcctttctacaaccaggatcatgagaaactctttgaattaatactaatggaagacattaaattcccccgaacactctcttcagatgcaaaatcattgctttcagggctcttgataaaggatccaaataaacgccttggtggagggccagatgatgcaaaagaaatcatgaggcatagttttttttctggagtaaactggcaagatgtatatgacaaaaagcttgtacctccttttaagcctcaagtaacatctgaaacagacacccgatattttgatgaagaatttacagctcagactattacaataacaccacctgaaaagtatgacgacgacggcatggacggcatggacagcgagcggcggccacacttccctcagttctcctactctgcaagcggacgggaataagttcctttcagtctgtttctacactgtcatcttagactttgcctgagactgattcctggacatctctaccagtcctcgctcttacagttagcaggggcaccttctgacatccctgaccagccaagggtcttcaccctcaccacctttcactcacatgaaaccatatacacagacactccagttttgtttttgcatgaaattgtatctcagtctaaggtctcatgctgttgctgctactgtcttactattatagcaaccttcagaagtaatttcacaatctttgggagtcatgagcccattgttcatttgtgcatcaagtgtcatcttttggtttttggttttccctagcagtgaaggctaaatgagatacactgattctaggtacattgttaactttctaggagataaatcaagaactaattagactaagaagatttagtttatatttctgaacaagcaattgttgaagggtggtggtgtgtgtatgtaagagtttagcagtgtatgtctgaccagcacatgaagtgtgtatcactgacatttggctggaaaatcttaggaaacattgagaggactgaagggccaatgagatacacatttttttttttttaaatcctgctggctattgtggaggaagcccagtgccacccatggatgcccaggtttcaagggcaaatggacattgttcaaccaagtactgacggtgcagagtcccctagagagtcttaggatctactttgtaccagagagtattttcccctgggcttggcaacctttttcccttctttatatttatcacctctgatggctgaagaatgtagacagtataatgatcctgctgtcgccaaaatctatacaccaaggtgaacaggtgtttgccttaccagttttggagtttttactttcagttataggtaaattagctttttctcagatgttaaaaccttgaatgtcctttatggttttgtttatattacagtagtatttattttttagtggtaagaattgtgttgtcatgttagcaagcgcagctccaattcacagatcattgcctgcgttttcttttgacccatgtgcaaggaatgtacacacccattagaatcatgcactttttctcctatgttaaaaacaatggtaaggctctgggactgtatctgttggttagaatctggctagaatctctgggagaagagatgctgtgatttaaccccttggagctgaaatgagagcatccccaactacccagtgccaagggatggatgtgagaggatagtctggcagaaagtaatggagaaagctgagacccaaatgatgatgagaaatgaactctactcggcaaaatgaacacatcacaagtattcacacagtaggaagtaaaggataagaagctgatgtgtttattcttatcaaggtacattcattactgaatcaagccatcagggaaaacaacaaaacaaaacacctctagcttttatttttctgtacatattcatgtctcccaacccaaacatttctcactgagaggggacatgtatgcaaacctcatctttctccttcattaatgatgatcttcagattaaccctttggtgctaggagctgacactttccaaagcagactatgacgtccaaggatcaaagcctctcttgcagcaagcaagagcaactgctgctccaaggcgtgatcatggaagtggaagaccacagtgtgacacagtggtactgcattgtctttttcagtaggaagtcatgttctcatgcaagtgtcttggcctctaggctcagacttagagaagcagagccgctgctggcagtggctggccgctgggactgctggggtgtaaagggcattttcctaaggcagctaagacagactcagacatagatttttgtcctaatttttcatgtttctacctgggtgagtttaccctcagtgggaagtaggaagttaaacaggtgtattcttacatctgctgtcttctgttttcctacatagaagtatgttgaatgttgcacattgacaaggaagtagattagatggtaagtccttttaagttaccctgcatccattctgtaagaaaggaggaggagaggaagaaaagcaagctgagcctggccgatgttcagaacatttactccctcagagggagcagacactgtacactaagcacacagcttcttttgttttgtctctccttccaagtcttaaggtaatttcatgaccttggccaaggatactttgtgaagattaatgaggggacattgacaatgcctcaggctggccactcctcacactggcccttggtccccagccattcaaggggttactcatcccatccctgactacctgagtgtcactacaggtgggttcttggctttgagttcaagaattctttccttacaccctccaccagatgtcagtggaaaggcagaagtagaaccgaggtgtgggaggagattctaaaagcttgtgttgcccaggcctctagcttagggttctggcaattgagacggcctgaaacctgacagcatcatgtccgggcagctctggctttgtgagtccagctcctaccaactccaccttgacctcttgtctccatgcagggctgtctgggaggaaactggccacttctgctcagactgctgccagcgctctcacagctttgctctctacactaatgacatagattattccagtattgttcccatttcccacctgacctccagcttctcggagctgacttcttgcaggggccacatgcttctttccctcactaactgcagggtctccaccacacctcagtgtacacactttgctgctaccgtctgtactgtctacatcacggttcccttagcttgctcctggtagtgcattacaggcaagcatgaaatgtaaagtatttatttaaataaaaaggaaacctctgaattggtcatcgagtcacctccctgtagatttgtagtctgtgacatatttcgactttctagtcctgctagatccatataagatctggtcatctggttaagggtttgaagcagacatagctatgaacccggatggtatcatcactgtcacggctggccacactacagatgtttatgtccttacgtttatgacccacagggtgtgaagtaacttccaagaacctgattttgtacactgtccactctcagcatctcacactctctgatagggacacacattacttccttcgtacagacagcttaaataaagccctatgtcaatctgcaaaaaaaaaaaaaaaa

SEQ ID NO: 30 - protein, 152 aa, Mus musculus
MRFCLFSFALIILNCMDYSQCQGNRWRRNKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLDETMECVEGCEVGHWSE

SEQ ID NO: 31 - protein, 196 aa, Mus musculus
MAPGPSATQGILLLLPLLPLSQVTLGSADRNCDPSDQCPPQARWSSLWHVGLILLAILLMLLCGVTASCVRFCCLRKQTHTQSHTPAAWQPCDGTVIPVDSDSPAHSTVTSYSSVQYPLGMRLPLYFGEPDPDSMVPPTYSLYASELPPSYDEVVKMIKAREEVAAPSEKTNSLPEALEPETTGGPQEPGPSAQRP

SEQ ID NO: 32 - protein, 340 aa, Mus musculus
AILHALELLLIRNYSPKRSFFIALGHDEEVSGEKGAQKISALLQARGVQLAFLVDEGSFILEGFIPNLEKPVAMISVTEKGALDLMLQVNMTPGHSSAPPKETSIGILSAAVSRLEQTPMPNMFGGGPLKKTMKLLANEFSFPINIVLRNLWLFHPIVSRIMERNPITNALVRTTTALTMFNAGIKVNVIPPLAQATINCRIHPSQTVHEVLELVKNTVADDRVQLHVLRSFEPLPISPSDDQAMGYQLLQETIRSVFPEVDIVVPGICIANTDTRHYANITNGMYRFNPLPLNPQDFSGVHGINEKVSVQNYQNQVKFIFEFIQNADTYKEPVPHLHEL

SEQ ID NO: 33 - DNA, 2304 bp, Mus musculus
ggtggcttttggggagagacatggctgagctacttgctagcttgcccgcctgggcagctgtgctccttctctttttcgctacggtctccggatccactggccctagaagcagggaaaatcggggggcgtcccggatcccttcccagttcagcgaggaggagcgtgtcgctataaaagaggcgctgaaaggtgccatccagattcccacagtgtctttcagccacgaggaatccaacaccacagcccttgctgagtttggagaatatatccgcaaagccttccctacagtgttccacagcagccttgtccaacatgaagtcgtggcaaagtatagccacctgttcaccatccaaggctcagaccccagtttgcagccctacatgctgatggctcacattgatgtggttcctgccccggaagaaggatgggaggtgcccccgttctcaggcctggaacgcaatggcttcatctatggccggggtgcgctggacaacaaaaactctgtgatggcgatcctgcatgctttggagctcctgttgatcagaaactacagccccaaaagatctttcttcattgctttgggccatgatgaggaggtgtccggggaaaagggggctcagaagatctcagcactcttacaggcaaggggtgtccagctagccttccttgtggatgaagggagctttatcttggaaggcttcattccaaacctcgagaagccagttgccatgatttcagtcactgagaagggtgcccttgacctcatgctgcaagtaaacatgactccaggccactcttcagctcccccaaaggagacaagcattggcattctttctgccgctgtcagccgactggagcagacaccaatgccgaatatgtttggaggagggccattgaagaagacaatgaagctactggcaaatgagttttccttccctatcaatatagtcttgagaaacctgtggctatttcatcccattgtgagcaggataatggagaggaaccccataacaaatgcgctggtccgaactaccacagccctcaccatgttcaatgcaggaatcaaggtgaatgtcatccctccattggctcaggctacaatcaactgccgaattcacccttcgcagacagtacatgaggtcctagaacttgtcaagaacaccgtggctgatgacagagtccagctgcatgtgttgagatcctttgaacccctgcccatcagcccctctgatgaccaggccatgggctaccagctgcttcaagagaccatacgatctgtcttcccggaagtcgacatcgtcgtccccggtatttgtattgccaatacggacacccgacactatgccaacatcaccaatggcatgtaccggttcaacccccttcccctgaaccctcaggacttcagtggtgtccatggaatcaatgagaaagtttccgttcagaactaccagaaccaggtgaagttcatctttgagttcatccaaaatgccgacacttacaaagagccagttcctcatctgcatgaactatgagctgagacttcatagttgaatgcgacagagaactgaaagaacgctaagatgagggagcagctggcacacaagatcatctgaaaacagcagtgttagatcgggcctcctacatcagggacagaagagaagttcagcaaagaccctttcttgggtcctgtcttttgttccttctacctaagccttgtccaggattccctatctcctaggcactgtgatacatcaaggccatcggtgctgatctaactagcctgtgaaataagctatgcagtagaacaagtggaaaccagaggcggtgcaatggaaaacattatgcaaatgtgaaagttgttagtcattagggaaatgcaaattttaactaccccgagaaatcactttatattcaccaggttggtcgtaatcaaaaaaaaaaaaaaatggaaaataacaaatgttggcaaggatatagagacattggaagcctcgtgcattcgctggtgggaatgtgaaatggcacagcagctgtggagtcagctgtggaaacggttggtgtttcctttgaagttaaatggagaattaccgtattaccctgcaattccacttcaaagcgtacggtcaagagaaatgaaaacaacaggtgttcaaacacctttttgttgacccaaaaatgacatcacagaacttttttaagtacaaaaacttttatgtatacaattaaaaatatatacatgtctatgctcaaagactttgtaatgtaaattgggagataaaataaatgtaataaaacaagatgttc

SEQ ID NO: 34 - protein, 503 aa, Mus musculus
MAELLASLPAWAAVLLLFFATVSGSTGPRSRENRGASRIPSQFSEEERVAIKEALKGAIQIPTVSFSHEESNTTALAEFGEYIRKAFPTVFHSSLVQHEVVAKYSHLFTIQGSDPSLQPYMLMAHIDVVPAPEEGWEVPPFSGLERNGFIYGRGALDNKNSVMAILHALELLLIRNYSPKRSFFIALGHDEEVSGEKGAQKISALLQARGVQLAFLVDEGSFILEGFIPNLEKPVAMISVTEKGALDLMLQVNMTPGHSSAPPKETSIGILSAAVSRLEQTPMPNMFGGGPLKKTMKLLANEFSFPINIVLRNLWLFHPIVSRIMERNPITNALVRTTTALTMFNAGIKVNVIPPLAQATINCRIHPSQTVHEVLELVKNTVADDRVQLHVLRSFEPLPISPSDDQAMGYQLLQETIRSVFPEVDIVVPGICIANTDTRHYANITNGMYRFNPLPLNPQDFSGVHGINEKVSVQNYQNQVKFIFEFIQNADTYKEPVPHLHEL

SEQ ID NO: 35 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 36 - DNA, 703 bp, Homo sapiens
gcctggggtcacagggatgccgcggctcctccgcttgtccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcattttttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacattta

SEQ ID NO: 37 - DNA, 2794 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 38 - DNA, 2878 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 39 - protein, 1686 aa, Homo sapiens
MAQIFSNSGFKECPFSHPEPTRAKDVDKEEALQMEAEALAKLQKDRQVTDNQRGFELSSSTRKKAQVYNKQDYDLMVFPESDSQKRALDIDVEKLTQAELEKLLLDDSFETKKTPVLPVTPILSPSFSAQLYFRPTIQRGQWPPGLPGPSTYALPSIYPSTYSKQAAFQNGFNPRMPTFPSTEPIYLSLPGQSPYFSYPLTPATPFHPQGSLPIYRPVVSTDMAKLFDKIASTSEFLKNGKARTDLEITDSKVSNLQVSPKSEDISKFDWLDLDPLSKPKVDNVEVLDHEEEKNVSSLLAKDPWDAVLLEERSTANCHLERKVNGKSLSVATVTRSQSLNIRTTQLAKAQGHISQKDPNGTSSLPTGSSLLQEVEVQNEEMAAFCRSITKLKTKFPYTNHRTNPGYLLSPVTAQRNICGENASVKVSIDIEGFQLPVTFTCDVSSTVEIIIMQALCWVHDDLNQVDVGSYVLKVCGQEEVLQNNHCLGSHEHIQNCRKWDTEIRLQLLTFSAMCQNLARTAEDDETPVDLNKHLYQIEKPCKEAMTRHPVEELLDSYHNQVELALQIENQHRAVDQVIKAVRKICSALDGVETLAITESVKKLKRAVNLPRSKTADVTSLFGGEDTSRSSTRGSLNPENPVQVSINQLTAAIYDLLRLHANSGRSPTDCAQSSKSVKEAWTTTEQLQFTIFAAHGISSNWVSNYEKYYLICSLSHNGKDLFKPIQSKKVGTYKNFFYLIKWDELIIFPIQISQLPLESVLHLTLFGILNQSSGSSPDSNKQRKGPEALGKVSLPLCDFRRFLTCGTKLLYLWTSSHTNSVPGTVTKKGYVMERIVLQVDFPSPAFDIIYTTPQVDRSIIQQHNLETLENDIKGKLLDILHKDSSLGLSKEDKAFLWEKRYYCFKHPNCLPKILASAPNWKWGNLAKTYSLLHQWPALYPLIALELLDSKFADQEVRSLAVTWIEAISDDELTDLLPQFVQALKYEIYLNSSLVQFLLSRALGNIQIAHNLYWLLKDALHDVQFSTRYEHVLGALLSVGGKRLREELLKQTKLVQLLGGVAEKVRQASGSARQVVLQRSMERVQSFFQKNKCRLPLKPSLVAKELNIKSCSFFSSNAVPLKVTMVNADPLGEEINVMFKVGEDLRQDMLALQMIKIMDKIWLKEGLDLRMVIFKCLSTGRDRGMVELVPASDTLRKIQVEYGVTGSFKDKPLAEWLRKYNPSEEEYEKASENFIYSCAGCCVATYVLGICDRHNDNIMLRSTGHMFHIDFGKFLGHAQMFGSFKRDRAPFVLTSDMAYVINGGEKPTIRFQLFVDLCCQAYNLIRKQTNLFLNLLSLMIPSGLPELTSIQDLKYVRDALQPQTTDAEATIFFTRLIESSLGSIATKFNFFIHNLAQLRFSGLPSNDEPILSFSPKTYSFRQDGRIKEVSVFTYHKKYNPDKHYIYVVRILWEGQIEPSFVFRTFVEFQELHNKLSIIFPLWKLPGFPNRMVLGRTHIKDVAAKRKIELNSYLQSLMNASTDVAECDLVCTFFHPLLRDEKAEGIARSADAGSFSPTPGQIGGAVKLSISYRNGTLFIMVMHIKDLVTEDGADPNPYVKTYLLPDNHKTSKRKTKISRKTRNPTFNEMLVYSGYSKETLRQRELQLSVLSAESLRENFFLGGVTLPLKDFNLSKETVKWYQLTAATYL

SEQ ID NO: 40 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

(19) United States
(12) Patent Application Publication
(10) Pub. No.: US 2011/0191871 A1
(43) Pub. Date: Aug. 4, 2011

(54) METHODS TO IDENTIFY FACTORS ASSOCIATED WITH MUSCLE GROWTH AND USES THEREOF

(75) Inventors: Kenneth Walsh, Carlisle, MA (US); Noriyuki Ouchi, Boston, MA (US); Yasuhiro Izumiya, Boston, MA (US)

(73) Assignee: TRUSTEES OF BOSTON UNIVERSITY, Boston, MA (US)

(21) Appl. No.: 12/281,115

(22) PCT Filed: Feb. 23, 2007
(86) PCT No.: PCT/US07/04835

§ 371 Date: Aug. 28, 2008

Related U.S. Application Data
(60) Provisional application No. 60/777,654, filed on Feb. 28, 2006.

(51) Int. Cl. C12Q 1/68; C12N 5/10; A01K 67/00

(52) U.S. Cl. 800/13; 435/6.1; 435/325; 435/6.18

(57) ABSTRACT

The present invention relates to methods to identify factors associated with muscle growth, angiogenesis, obesity, insulin sensitivity body weight, fat mass, muscle mass and cardiovascular function. In particular, the methods of the present invention relates to assays to identify such factors using a transgenic animal model and/or a cell-based assay.

### CROSS REFERENCE TO RELATED APPLICATIONS

This application claims the benefit under 35 U.S.C. 119(e) of U.S. Provisional Patent Application Ser. No. 60/777,654, filed Feb. 28, 2006, the contents of which are herein incorporated by reference in their entirety.

### FIELD OF THE INVENTION

This invention is directed to assays to identify factors and proteins involved in muscle growth, in particular using a transgenic animal model assay and/or cell-based assays.

### BACKGROUND

Systemic muscle atrophy occurs upon fasting and in a variety of diseases such as cachexia, cancer, AIDS, prolonged bed rest, and diabetes (1). One strategy for the treatment of atrophy is to induce the pathways normally leading to skeletal muscle hypertrophy.

Skeletal muscle hypertrophy plays an important role in normal postnatal development and the adaptive response to physical exercise (2). This process is associated with blood vessel recruitment, such that capillary density is either maintained or increased in the growing muscle (3-6). Conversely, myofiber atrophy that occurs with aging, disuse, and myopathic disease is associated with capillary loss (7, 8). It has been shown that angiogenesis and compensatory muscle hypertrophy are temporally coupled, suggesting that these two processes may be controlled by common regulatory mechanisms (9).

Muscle accumulation inversely correlates with fat mass. For example, skeletal muscle-specific expression of IGF-1 (10), ski (11) or Akt1 (12). However, the hormonal regulatory mechanisms by which skeletal muscle controls lipolysis and fatty acid mobilization and uptake by muscle is poorly understood. Furthermore, proper skeletal function is also important for maintenance of normal glucose metabolism (13-15). Skeletal muscle resistance to insulin-stimulated glucose uptake is the earliest known manifestation of non-insulin-dependent (type 2) diabetes mellitus (16, 17). Increasing skeletal muscle activity can improve insulin sensitivity and prevent the progression from impaired insulin tolerance in most type 2 diabetic patients (18).

Collectively, these data show that increasing muscle mass is associated with angiogenesis, fat mass reduction and improved insulin sensitivity. However the molecules secreted by growing muscle that orchestrate these processes are largely unknown. Identification of such proteins is useful in that manipulation of expression of such gene products may allow for the treatment or prevention of various diseases and/or disorders.

Akt is a serine-threonine protein kinase that is activated by various extracellular stimuli through the phosphatidylinositol 3-kinase (PI 3-kinase) pathway (19). Numerous studies have implicated Akt signaling in the control of organ size and cellular hypertrophy (20, 21). With mammalian cell cultures, it has been shown that an oncogenic Akt-Gag fusion protein promotes glucose transport and protein synthesis in L6 myotubes (22) and that constitutive activation of Akt signaling promotes a hypertrophic phenotype in muscle both in vitro (23) and in vivo (24). Similarly, Akt signaling has also been shown to control the smooth muscle cell hypertrophy that is associated with hypertension (25, 26), and PI 3-kinase signaling has been implicated in cardiac myocyte hypertrophy (27). It has been shown that myogenic Akt signaling regulates blood vessel recruitment during skeletal myofiber growth in vitro and in vivo (28). Hypertrophy of cultured C2C12 myotubes in response to insulin-like growth factor 1 or insulin and dexamethasone results in a marked increase in the secretion of vascular endothelial growth factor (VEGF). Myofiber hypertrophy and hypertrophy-associated VEGF synthesis were specifically inhibited by the transduction of a dominant-negative mutant of the Akt1 serine-threonine protein kinase. Conversely, transduction of constitutively active Akt1 increased myofiber size and led to a robust induction of VEGF protein production. The activation of Akt1 signaling in normal mouse gastrocnemius muscle was sufficient to promote myofiber hypertrophy, which was accompanied by an increase in circulating and tissue-resident VEGF levels and high capillary vessel densities at focal regions of high Akt transgene expression. In a rabbit hind limb model of vascular insufficiency, intramuscular activation of Akt1 signaling promoted collateral and capillary vessel formation and an accompanying increase in limb perfusion. These data suggest that myogenic Akt signaling controls both fiber hypertrophy and VEGF synthesis, illustrating a mechanism through which blood vessel recruitment can be coupled to normal tissue growth. However, it is likely that other as-yet-unidentified angiogenic regulatory factors also participate in this process.

There is a need for the identification of muscle secreted factors that participate in the process of muscle growth and may also influence angiogenesis, insulin sensitivity and fat mass reduction.

### SUMMARY OF THE INVENTION

The present invention relates to methods to identify factors associated with muscle growth angiogenesis, obesity, insulin sensitivity and cardiovascular function based on the expression of muscle related proteins in muscle cells. In particular, one aspect of the present invention relates to assays to identify such factors using a transgenic animal model. Another aspect of the present invention relates to assays identifying such factors using a cell-based assay.

The inventors have discovered that exogenous expression of a muscle related protein, for example the active isoform of Akt1 in skeletal muscle induces muscle growth, e.g., hypertrophy. Cells containing an active isoform of Akt1, i.e an Akt1 isoform wherein activation by upstream factors are unnecessary, under the control of an inducible muscle-specific promoter can be utilized to identify factors associated with muscle growth. Identification of secreted factors whose expression is induced by muscle growth is, for example, useful for the treatment of muscle related disorders. Such secreted factors may be administered to subjects in order to promote muscle growth. Furthermore, muscle growth is associated with angiogenesis, insulin sensitivity and fat mass regulation. Accordingly, identification of factors associated with muscle growth also provides factors associated with angiogenesis, insulin sensitivity and/or fat mass regulation. These factors can be used for a range of therapies, such as for example, treating obesity, angliogensis disorders, muscle disorders etc. Using, for example, a transgenic animal expressing Akt1, the inventors have identified a range of secreted factors which induce muscle growth and are useful in the treatment of obesity, angiogenesis disorders insulin-related disorders and muscle degenerative disorders.

Accordingly, the present invention provides a method for identifying factors associated with muscle growth and/or participate in the process of muscle growth. Another aspect of the invention, method are provided for identifying factors associated with muscle growth that influence angiogenesis, insulin sensitivity and fat mass reduction.

Factors include proteins, e.g., secreted proteins, membrane bound proteins, etc., functional RNAs, e.g., microRNAs, ribozymes, etc.

In some embodiments, the present invention provides methods for identifying muscle secreted factors or proteins, herein referred to MSP (muscle secreted proteins) that participate in the process of muscle growth. In other embodiments, factors associated with muscle growth and/or participate in the process of muscle growth are receptors and/or intracellular signaling molecules involved in muscle growth, angiogenesis, insulin sensitivity or fat mass reduction.

One aspect of the present invention relates to the use of muscle cells comprising a muscle related transgene. In some embodiments, the muscle related transgene comprises a nucleic acid construct, wherein said construct comprises is a nucleic acid sequence encoding a muscle related gene operatively linked to a muscle promoter. In some embodiments, the muscle related gene is a positive regulator of muscle growth, for example but not limited to a constitutively-active form of muscle related protein, for example but not limited to constitutively active Akt1. In some embodiments, the muscle related protein is a protein that positively regulates muscle growth, for example an active form of Akt1, Akt2, Akt3 or PI-3 kinase or homologues or variants thereof. In some embodiments, the muscle promoter is a smooth muscle or skeletal muscle promoter, and in some embodiments the muscle related proteins are operatively linked to an inducible muscle-specific promoter.

In alternative embodiments, the muscle related transgene comprises a nucleic acid construct, wherein said construct comprises a nucleic acid sequence encoding an inhibitor to a muscle related gene operatively linked to a muscle promoter. In such an embodiment, the muscle related gene is a negative inhibitor of muscle growth, for example but not limited to myostatin and variants thereof. In such embodiments, the inhibitor is a nucleic acid inhibitor, for example a RNAi, siRNA, shRNAi, miRNA, antisense nucleic acid, antisense oligonucleotide (ASO) or neutralizing antibody or fragments or analogues thereof.

The present invention provides methods to identify factors associated with muscle growth, influence angiogenesis, insulin sensitivity and fat mass reduction, using gene and/or protein expression analysis.

In one embodiment, the method of the present invention compares the expression profile of muscle cells comprising a muscle related transgene and expressing such a transgene for a period of time with an expression profile of muscle cells not comprising and/or not expressing the muscle-related transgene. The expression profile can be done by gene expression analysis or protein expression analysis. Genes and/or proteins that are identified as differentially expressed between muscle cells comprising and expressing the muscle-related transgene for a period of time and muscle cells not comprising and/or expressing the muscle-related transgene are identified as coding for factors associated with muscle growth, angiogenesis, insulin sensitivity and fat mass reduction.

Such factors may be further selected, e.g., by electronic sequence analysis of the genes. Factors include proteins, e.g., secreted proteins, membrane bound proteins, etc., functional RNAs, e.g., microRNAs, ribozymes, etc. The muscle cells that do not express the transgene may comprise the transgene in their genome. In some embodiments, the muscle cells comprising and/or expressing the muscle related transgene can express the transgene for specific period of time, for example muscle cells can constitutively or conditionally express such a transgene, or express such a transgene for period of time before and/or after a period of repressed transgene expression

In some embodiments, a secondary expression analysis may be performed, for example secondary gene expression and/or secondary protein expression analysis. The secondary expression analysis further utilizes (i) muscle cells comprising and expressing the muscle-related transgene and (ii) muscle cells comprising the muscle related transgene, wherein the expression of the muscle-related transgene in the muscle cells is repressed for a period of time subsequent to expression of the muscle-related transgene. In other embodiments, the expression of the muscle related transgene in muscle cells of (ii) is repressed but the muscle related transgene was expressed for a period of time prior to its repression. In some embodiments, the muscle cells have expression or are repressed for expression of the muscle related transgene for specific periods of time, for example, about 1 hr, about 2 hrs, about 6 hrs, about 12 hrs, about 24 hrs, about 2 days, about 3 days, about 4 days, about 5 days, etc.

The set of genes or proteins differentially expressed between muscle cells expressing the muscle related transgene and muscle cells repressed for expression of the muscle related transgene subsequent to expression of the muscle related transgene are compared to the set of genes or proteins differentially expressed between muscle cells expressing, at least for a period of time, the muscle related transgene and muscle cells not expressing the muscle related transgene. Genes differentially expressed can be identified as factors associated with muscle growth. Additionally, tests can be readily performed to confirm the function.

Comparisons of genes and/or protein expression profiles between muscle cells (i) comprising and expressing the muscle related transgene or (ii) comprising and not expressing the muscle related transgene, or (iii) not comprising or not expressing the muscle related transgene may take place using cells of various genetic backgrounds. For example, identification of factors associated with muscle growth by determination of differentially expressed genes and/or proteins may utilize muscle cells derived from animals, for example, with predisposition to diabetes, with angiogenesis defect, with a muscle disorder, with a disruption of gene associated with muscle growth, etc. Comparison may be made between cells of the same genetic background or different genetic backgrounds. In some embodiments, the cells are muscle cells, for example skeletal muscle cells, and in some embodiments the muscle cells are smooth muscle cells. In alternative embodiments, any cell can be used, for example cells from tissues or organs from a transgenic animal comprising and expressing at for at least a period of time the muscle related transgene of the invention. In such embodiments the cells are, for example, liver cells, adipose cells, cardiac cells, muscle cells, brain cells, skin cells and the like.

In some embodiments of the present invention, the muscle cells useful in the identification of such factors and/or proteins that affect muscle growth, angiogenesis, obesity, insulin sensitivity and cardiovascular function are muscle cells comprising the muscle related transgene of the present invention. In some embodiments, the muscle cells are cultured muscle cells, for example, a muscle cell line, for example but not limited to C2C12 cells. In alternative embodiments, the muscle cells are skeletal muscle cells and in alternative embodiments the muscle cells are smooth muscle cells. In another embodiment, the muscle cells are derived from a mammal, for example primary muscle cells and/or myocytes. In some embodiments, the muscle cells are human muscle cells or animal muscle cells, for example transgenic animal muscle cells. In an alternative embodiment, the muscle cells can be part of an animal, for example, the muscle cells are part of a transgenic animal, for example but not limited to, the muscle cells are part of a transgenic animal expressing the muscle related transgene of the present invention.

Another aspect of the present invention relates to an assay using a transgenic animal to identify such proteins that affect muscle growth, angiogenesis, obesity, insulin sensitivity and cardiovascular function. The factors can be used to treat various disorders, such as for example, obesity, muscle disorders, insulin related disorders, angliogenesis disorders etc.

Accordingly, also encompassed in the present invention is a transgenic animal expressing the muscle related transgene of the present invention. This transgenic animal may be useful in the identification of additional factors which affect muscle growth, angiogenesis, insulin sensitivity and fat accumulation.

In some embodiments, the transgenic animal comprises a nucleic acid construct of a muscle related transgene, where the muscle related transgene is a nucleic acid sequence encoding a muscle related gene operatively linked to a muscle promoter, for example a skeletal muscle promoter or a cardiac muscle promoter. In some embodiments, the muscle related gene is a constitutively-active form of muscle related protein, for example but not limited to a constitutively active isoform of Akt1, Akt2, Akt3 or PI-3 kinase. In some embodiments, the muscle related protein is a protein that positively regulates muscle growth, for example Akt1, Akt2, Akt3 or PI-3 kinase or homologues or variants thereof. In some embodiments, the muscle promoter is a smooth muscle or skeletal muscle promoter, and in some embodiments the muscle related protein is operatively linked to an inducible muscle-specific promoter.

The muscle related transgene comprising a nucleic acid or gene encoding a protein associated with muscle growth, for example but not limited to Akt1, e.g., constitutively active isoform of Akt1, may be operatively linked to a muscle specific inducible promoter. One can use any cell, preferably a muscle cell. In one embodiment, the cell is grown in a cell culture. In another embodiment, the cell is present in an animal, for example a transgenic animal. The transgenic animal may comprise one or more exogenous DNAs or transgenes. The one or more transgenes may be operatively linked. The transgenic animal may contain a transgene wherein an inducible first promoter regulates transcription of a muscle related protein, for example a constitutively active isoform of Akt1. The transgenic animal may further contain a transgene wherein a muscle-specific second promoter regulates transcription of an inducer of the first promoter. The inducible first promoter may require for transcription both the inducer transcribed from the second promoter and an additional exogenously added factor. Thus, addition of the exogenously added factor enables expression of the muscle related protein under the control of an inducible muscle-specific promoter.

The transgenic animal containing a muscle related transgene, for example a positive regulator of muscle growth under the transcriptional control of an inducible muscle promoter, for example transgenic animals with, for example but not limited to muscle-specific inducible Akt1, may be useful for the derivation of cell lines, e.g., muscle cell lines, e.g., skeletal muscle cell lines. The transgenic animal may further contain additional transgenes. The additional transgenes may comprise the factors identified by the methods of the present invention.

In one embodiment, the present invention does not include expression of the muscle related transgene in muscle cells, for example smooth muscle. For instance, also encompassed within the present invention is identification of factors important in muscle growth, angiogenesis, muscle regeneration and fat mass, by administering an effective amount of an agent that functions as an inhibitor to a muscle related gene, wherein the muscle related gene is a negative regulator of muscle growth, for example but not limited to myostatin. An inhibitor for use in such an embodiment is, for example but not limited to, a neutralizing antibody to myostatin or a small molecule antagonist or a nucleic acid inhibitor to myokatin, for example but not limited to a RNAi or antisense oligonucleotide. In other words, the present invention also provides methods to identify factors associated with muscle growth, angiogenesis, muscle regeneration and fat mass, using cells and/or animals not comprising the muscle related transgene of the present invention, where the method involves administering an agent or inhibitor of a negative regulator of muscle growth. Accordingly, the expression profile of cells administered an agent or inhibitor of a negative regulator of muscle growth is compared to cells not administered such an agent or inhibitor, and the differentially regulated genes identify genes and/or gene products important in muscle growth, angiogenesis, muscle regeneration and fat mass are identified.

Using the methods of the present invention, one may determine if the muscle related transgene produces genes and/or gene products identified by the methods of the present invention ameliorate the symptoms and/or cure a disease or disorder. For instance, the genes and/or gene products identified by the methods of the present invention, or agonists thereof, can be assessed in models of angiogenesis, obesity, glucose intolerance and insulin tolerance and muscle degeneration and an improvement in symptoms of the disease or disorders identifies potential therapeutic targets for such disorders.

### BRIEF DESCRIPTION OF THE FIGURES

FIGS. 1A-C show generation of skeletal muscle-specific conditional Akt1 TG mice. (FIG. 1A) Schematic illustration of binary TG system. FIG. 1B shows DOX-dependent expression of Akt1 transgene. Top: Temporal profile of DOX treatment. Bottom: Western blot analysis of transgene expression in gastrocnemius muscle. FIG. 1C shows western blot analysis of transgene expression in different tissues.

FIGS. 2A-D shows conditional activation of Akt1 in skeletal muscle caused reversible muscle hypertrophy. FIG. 2A Top shows Temporal profile of DOX treatment. FIG. 2A Bottom shows representative gross appearance of the DTG mice. FIG. 2B Top shows time course of transgene expression. FIG. 2B bottom shows Time course of gastrocnemius muscle weight. Results are presented as mean±SEM (n=4-12 each) *P<0.05 vs. day 0; #P<0.05 vs. day 14. FIG. 2C shows histological analysis. Top: H&E staining of gastrocnemius muscle sections. Scale bars: 100 μm. Bottom, Left: Distribution of mean cross-sectional areas of muscle fibers. Right: Mean cross-sectional areas of muscle fibers. Results are presented as mean±SEM (n=6). *P<0.05 vs. control. FIG. 2D Top shows Representative western blot and histological analysis of gastrocnemius muscle in DTG 2 or 6 weeks after Akt1 activation. Scale bars: 100 μm. Bottom: Gastrocnemius muscle weight and mean cross-sectional areas of muscle fibers. Results are presented as mean±SEM (n=6). *P<0.05 vs. control.

FIGS. 3A-C shows Akt1-mediated type II muscle growth led to increase peak force output. FIG. 2A shows representative images of gastrocnemius sections from control and DTG mice 2 weeks after Akt1 activation stained with anti-HA antibody and MHC isoform antibodies, for MHC type I, MHC Type IIa and MHC Type IIb, with FIG. 1A showing Akt1 transgene expression induces the growth and hypertrophy of MHC Type IIb fibers (panels) and quantified in the histogram. FIG. 3B shows Forearm grip strength measurements. Results are presented as mean±SEM (n=6) *P<0.05 vs. control. FIG. 3C shows forced treadmill exercise test, with the left graph showing Running time, and the right showing running distance. Results are presented as mean±SEM (n=4) *P<0.05 vs. control.

FIGS. 4A-D show Akt1-mediated type II muscle growth regressed diet-induced obesity. FIG. 4A Left shows representative gross appearance and ventral view of the control and DTG mice fed HF diet, and FIG. 4A Right shows body weight of control and DTG mice (n=12). FIG. 4B Left shows Representative MRI images of each group of mice which were shown at a level of the right renal pelvis, and FIG. 4B Right shows quantified measurements of total fat volume (n=4). FIG. 4C shows Histological analysis. H&E stained gastrocnemius muscle (Top) and white adipose tissue (Bottom) sections. Scale bars: 200 μm. FIG. 4D shows Gastrocnemius muscle and inguinal fat pad weight. Results are presented as mean±SEM (n=6). *P<0.05.

FIGS. 5A-D show Akt1-mediated type II muscle growth improved diet-induced severe insulin resistance. FIG. 5A shows blood glucose levels in Fasting (left) and fed (right) period. FIG. 5B shows fasting serum insulin levels. (FIG. 4C) Glucose tolerance tests. (FIG. 5D) Glucose uptake in vivo skeletal muscle. Results are presented as mean±SEM (n=8). *P<0.05 vs. HF diet fed control mice.

FIGS. 6A-C shows Akt1-mediated type IIb muscle growth led to increased energy consumption independent of food intake or activity level. (FIG. 6A) Food consumption was measured over 6 weeks after Akt1 activation in skeletal muscle (n=12 in each group). (FIG. 6B) Ambulatory activity levels (n=6 in each group). (FIG. 6C) left: 02 consumption (V02), right: respiratory exchange ratio measured by metabolic measuring system for 24 hours without food in control (white bars) and DTG (black bars) mice 4 weeks after DOX treatment (n=6 in each group). Results are presented as mean±SEM. DTG=MyoMouse.

FIGS. 7A-D shows Akt1-mediated type II muscle growth increased lipid oxidation in liver. FIG. 7A: Histological analysis. Oil red-O stained liver sections. Scale bars: 200 μm. FIG. 7B shows Total fatty acid β-oxidation of palmitic acid in liver. FIG. 7C shows fasting serum ketone body levels. FIG. 7D shows quantitative real-time PCR analysis in liver. Results are presented as mean±SEM (n=6).

FIG. 8 shows an alternative approach for the isolation of myokines: Myogenic cell lines transduced with an activated form of Akt.

FIG. 9 shows Akt1 induction by administration of Dox in drinking water leads predominantly to the hypertrophy of Type lib muscle fibers that are characterized as glycolytic/fast twitch. Less growth of oxidative/fast twitch muscle fibers (Type I, Type IIa) is evident.

FIG. 10A-F shows Akt-mediated type IIb muscle hypertrophy increased lipid oxidation in liver, but not muscle. FIG. 10A shows relative mRNA expression associated with fatty acid oxidation and mitochondrial biogenesis in gastrocnemial skeletal muscle (n=4 in each group. *P<0.05 vs. HF/HS diet fed control. ˜0.05 vs. normal diet fed control). FIG. 10B is Histological analysis. Oil red-O stained liver sections. Scale bars: 100 pm. FIG. 10C is Total fatty acid ˜3-oxidation of palmitic acid in liver (n=8 in each group). FIG. 10D s Expression of genes associated with fatty acid ˜3-oxidation in liver (n=4 in each group). (E) Serum (left) and urine (right) ketone body levels (n=9 to 12 in each group). FIG. 10F shows Serum lactate levels (n=6 in each group). Results are presented as mean±SEM. DTG=MyoMouse.

FIG. 11 shows evidence for satellite cell proliferation following Akt1 activation in MyoMice. FIG. 11A shows evidence for satellite cell proliferation at 2 weeks after transgene activation. BrdU incorporation into DNA was evident in histological sections of MyoMice 2, 4, 6, 8 and 10 weeks (w) after activation of Akt1, but not in control (cont) mice. Evidence for increased numbers of MyoD-positive satellite cells in MyoMice 2 weeks after transgene induction was also detected (not shown)

FIG. 12 is a schematic of tissues for differential gene expression analysis. Microarray analyses on muscle, liver and adipose tissues of diet-induced obese Akt1-mediated skeletal muscle expression (MyoMice) before and after Akt transgene expression. Analysis of such tissues enables identification of potential receptors and proteins secreted from liver and adipose tissue in response to skeletal muscle growth.

FIGS. 13A-C shows the effect of adenovirus expressing MSP3 on ischemia-induced angiogenic response in wild-type mice. FIG. 13B. shows a mouse hindlimb ischemia model, and Adenovirus-expressed MSP3 promotes blood vessel growth in the ischemic limb as monitored by Laser Doppler analysis (FIG. 13B) on legs and feet immediately before surgery and on postoperative days 0, 3, 7, 14, and 28 as illustrated in FIG. 11A. FIG. 11C shows intramuscular injection of an adenoviral vector expressing MSP3, but not MSP6 (FGF-21) or β-galactosidase, stimulates reperfusion in ischemic hindlimb of mice as assessed by laser Doppler analysis.

FIG. 14 shows the effect of Adv-MSP3 on capillary density in WT mice. Adenovirus-expressed clone 2 (MSP3) promotes microvessel formation as assessed by CD31-staining in histological section from ischemic limb. An adenoviral vector expressing FGF21 does not display this activity.

FIGS. 15A-D shows glucose tolerance test. MSP3 is a candidate metabolic regulator. Intramuscular injection of Adeno-MSP3 improves glucose sensitivity in a diet-induced obesity mouse model. FIGS. 15A and 15B shows adenovirus-encoded MSP3 appears functionally equivalent to adenovirus-delivered FGF-21 (also known as MSP6). FIGS. 15C and 15D shows MSP3 improves glucose sensitivity and metabolic response, which is not observed for other MSPS; MSP5, MSP2, MSP4 and MSP1. β-gal is the negative control.

FIG. 16 shows the full-length nucleotide sequence of MSP3. Nucleotide sequence of MSP3 showing “long” (SEQ ID NO: 1) and “short” (SEQ ID NO:2) alternatively-spliced forms.

FIG. 17 shows the position of MSP3 long (SEQ ID NO:1) and short (SEQ ID NO:2) and on chromosome 2.

FIG. 18 shows the alignments of MSP3 amino acid sequences between mouse (SEQ ID NO: 3), rat (SEQ ID NO: 4), and human (SEQ ID NO: 5). Amino acid sequence identity between mouse and rat is 94% and the sequence identity between mouse and human is 79%. The boxed area is the predicted signal sequence.

FIG. 19 shows PCR primers for detecting total expression of MSP3 (i.e for detecting both the long (SEQ ID NO:1) and short (SEQ ID NO:2) isoforms of MSP3. The location of the forward primer 3 is SEQ ID NO:10, and the reverse primer 3 is SEQ ID NO:11) are shown on the SEQ ID NO:1 and SEQ ID NO:2.

FIGS. 20A-B show tissue-specific expression profile of MSP3 in adult mouse tissues. FIG. 20A shows expression profile of total MSP3 (combined long and short forms) by RT-PCR. Expression in heart, brain, lung, thymus, lymph node, eye and skeletal muscle. Upregulation by Akt expression in C2C12 myogenic cells. FIG. 20B shows expression profile of MSP3 long and short forms in adult mouse tissues by RT-PCR.

FIG. 21 shows alternative splice isoform specific PCR Primers: Design of PCR Primers to differentially detect long and short forms of MSP3. Location and design of primers to detect long and short forms of (MSP3) clone 2 are shown, with the positions of Forward Primer 1 (SEQ ID NO:6), Forward Primer 2 (SEQ ID NO:8), Reverse Primer 1 (SEQ ID NO:9) and Reverse Primer 2 (SEQ ID NO:10) on the long form (SEQ ID NO:1) and short form (SEQ ID NO:2) of MSP3 shown.

FIGS. 22A-D shows MSP5 promotes angiogenesis in ischemic hind limb repair as shown by FIG. 22D by laser Dopper analysis. Effect of adenovirus expressing MSP5 (clone 5) on ischemia-induced angiogenic response in wild-type mice. FIG. 22D shows intramuscular injection of an adenoviral vector expressing MSP5, but not MSP1 or β-galactosidase, stimulates reperfusion in ischemic hindlimb of mice as assessed by laser Doppler analysis.

FIGS. 23A-B shows MSP5 promotes myofiber hypertrophy. FIG. 23A outline the protocol to assess MSP5-mediated hypertrophy of C2C12 myocytes in vitro was done by transfecting adenovirus expressing MSP5 or MSP3 or MyrAkt or β-gal 4 days after differentiation of C2C13 myocytes, and FIG. 23B shows the morphology of cells 4 days post-transfection. FIG. 23C shows quantitative analysis of myofiber width 4 days after transfection with Adv-expressing MSP5, MSP3, MyrAkt or β-gal (as shown in FIG. 23B) was examined microscopically. Transduction with MSP5 or myrAkt leads to detectable increases in myotube size, but adenoviral vectors expressing MSP3 or β-galactosidase has no effect.

FIG. 24 shows transduction with adenoviral vectors expressing MSP5 or myrAkt1 promotes 3H-leucine incorporation into protein as compared to baseline incorporation in the absence of virus or C2C12 cells transfected with Adenovirus expressing β-gal or MSP3. Representative results from duplicate experiments (left and right panels) is shown.

FIG. 25 shows MSP5 transfected C2C12 cells promote VEGF expression. Transduction of C2C12 cells with adenoviral vectors expressing MSP5 or myrAkt1, but not MSP3 (clone 2), activate VEGF expression in C2C12 cells.

FIGS. 26A-B shows Insulin-like 6 is regulated by Akt in muscle in vitro (FIG. 26B) and in vivo (FIG. 26A). FIG. 26A shows Insulin-like 6 transcript is dramatically upregulated 24-fold in MyoMice 2 weeks after transgene induction and FIG. 26B shows a 10-fold in C2C12 cells following transduction with Adeno-myrAkt1.

FIGS. 27A-D show that other relaxin family members, such as Insl3, Insl5, relaxin, Insl7 (relaxin 3) are not regulated following Akt transgene induction in MyoMice, as determined by RT-PCR of transcript expression levels.

FIGS. 28A-B shows Insulin-like 6 (Insl6) transcript is upregulated during muscle regeneration following cardiotoxin administration to tibialis anterius (TA) muscle. FIG. 28A shows Akt is also upregulated by this injury, whereas VEGF-A transcript is downregulated (upper panel). FIG. 28B shows other relaxin family members, Insl3, Insl5, relaxin, Insl7 (relaxin 3) are not regulated in cardiotoxin-injured mouse muscle (bottom panel).

FIGS. 29A-D shows Adenovirus-expressing insulin-like 6 (Insl6) does not affect C2C12 differentiation or hypertrophy. FIG. 29A shows C2C12 cells were transfected with Adv-Insl6 or βgal (Gal) at 240 multiplicities of infection (MOI) and morphology, and FIG. 29B shows the number of multi-nucleated myotubes, and FIG. 29E shows myotube width is not affected with transduction with Adv-Insl6. FIG. 29C shows Creatine kinase levels and FIG. 29D shows Leucine incorporation was also compared in C2C12 cells transfected with Adv-Insl6 or Adv-βgal control (Gal).

FIGS. 30A-B shows adenovirus-expressing insulin-like 6 stimulated the proliferation of rat skeletal muscle satellite cells. FIG. 30A shows Thymidine (³H-thymidine) incorporation is increased in Adv-Insl6 transfected cells compared to β-gal control transfected cells. FIG. 30 shows Western blot (WB) shows activation of satellite cell proliferation is accompanied by an increase in Rb protein (p-Rb) proliferation.

FIGS. 31A-B shows Insl6 facilitates TA muscle regeneration after cardiotoxin (CTX) injury. FIG. 31A shows administration of Adeno-Insl6 4 days after cardiotoxin administration improves tibialis arterius (TA) muscle regeneration compared to β-gal control. Improved regeneration is most notable at 7 and 14 days in histological sections (FIG. 31A). FIG. 31B shows at 7 days Insl6 overexpression repressed creatine kinase release into sera (lower left panel) which was not observed at 14 days (lower right panel).

FIG. 32 is similar to FIG. 31A, where administration of Adeno-Insl6 3 days after cardiotoxin (CTX) administration, Insl6 significantly promotes muscle regeneration of tibialis arterius (TA) 1 week following injury compared to β-gal control.

FIGS. 33A-D shows Insl6 reduces expression of TNFα and TNFβ1 and promotes collagen3 expression. Administration of Adeno-Insl6 results in a 200-fold increase in Insl6 expression FIG. 33A) in the muscle, and reduces TNFα (0.2 fold, p<0.03)(FIG. 33B), and TNFβ1 (0.9 fold, p>0.8) (FIG. 33D) and increases collagen 3 (1.8 fold, p>0.6) (FIG. 33D C) in muscle after injury. Insl6 and TNFα are 1 week post Adv-Insl6 injection (n=2)

### DETAILED DESCRIPTION OF THE INVENTION

The present invention is based on the discovery that skeletal muscle secretes factors that favorably affect muscle growth, angiogenesis, obesity, insulin sensitivity and cardiovascular function. The inventors have discovered methods to identify proteins that affect muscle growth, angiogenesis, obesity, insulin sensitivity and cardiovascular function, where one method of the present invention uses a transgenic animal model and another method of the present invention uses a cell-based assay.

In one aspect of the present invention, an assay using a transgenic animal model to identify such proteins that affect muscle growth, angiogenesis, obesity, insulin sensitivity and cardiovascular function is provided. In some embodiments, the transgenic animal model comprises a transgene comprising a muscle related protein operatively linked to a muscle promoter, for instance an inducible muscle-specific promoter. In some embodiments, the muscle related protein is, for example, a constitutively-active form of muscle related protein, where the muscle related protein positively regulates muscle growth. Such a positive regulator of muscle growth useful as a muscle related protein is, for example but not limited to the constitutively active Akt1 is expressed from an inducible promoter in muscle. The inventors have discovered that activation of such a transgene leads to muscle growth that is functionally stronger, and this muscle growth is accompanied by new blood vessel growth. Activation of such a transgene also induces muscle growth in animals, for example muscle hypertrophy of MHC Type IIb fibers and muscle regeneration. Furthermore, activation of such muscle related transgenes in obese animals, for example animals made obese by feeding a high-fat/high-sucrose diet leads to reductions in overall body weight and fat mass and in an improvement in insulin sensitivity. Accordingly, using such transgenic animals expressing a muscle related transgene that induces muscle growth, the inventors can identify factors that affect, for example but not limited to, muscle growth, fat mass, angiogenesis, body weight, and insulin and glucose sensitivity.

In another aspect of the present invention, a cell-based assay to identify such proteins that affect muscle growth, angiogenesis, obesity, insulin sensitivity and cardiovascular function is provided. In some embodiments, the cell-based assay comprises a muscle cell comprising a constitutively active-form of a muscle related protein, for example constitutively active Akt1 is expressed from an inducible promoter in muscle. In some embodiments, the muscle cell is a skeletal muscle cell, and in some embodiments the muscle cell is a primary muscle cells and in some embodiments, the muscle cell is a muscle cell line, for example a myogenic cell line for example, C2C12 cells. In some embodiments, the muscle cell is a human muscle cell.

In some embodiments, the constitutively active muscle related protein is, for example but not limited to, Akt1, Akt2, Akt3, PI-3 kinase, S6-kinase and mTOR or homologues or variants thereof.

In some embodiments, the muscle related gene is a positive regulator of muscle growth, for example but not limited to a constitutively-active form of muscle related protein, for example but not limited to constitutively active Akt1. In some embodiments, the muscle related protein is a protein that positively regulates muscle growth, for example Akt1, Akt2, Akt3, PI-3 kinase, S6-kinase and mTOR or homologues or variants thereof. In some embodiments, the muscle promoter is a smooth muscle or skeletal muscle promoter, and in some embodiments the muscle related proteins are operatively linked to an inducible muscle-specific promoter.

In alternative embodiments, the muscle related transgene comprises a nucleic acid construct, wherein said construct comprises is a nucleic acid sequence encoding an inhibitor to a muscle related gene operatively linked to a muscle promoter. In such an embodiment, the muscle related gene is a negative inhibitor of muscle growth, for example but not limited to myostatin and homologues and variants thereof. In such embodiments, the inhibitor is a nucleic acid inhibitor, for example a RNAi, siRNA, shRNAi, miRNA, antisense nucleic acid, antisense oligonucleotide (ASO) or neutralizing antibody or fragments or analogues thereof. In some embodiments, the muscle-related protein is myostatin or homologues or variants thereof.

The present invention further provides methods to identify factors secreted by muscle that control lipolysis and fat volume, satellite cell recruitment and muscle fiber growth, insulin sensitivity, bone growth and angiogenesis. Muscle cells comprising the transgene of the present invention encoding a gene associated with muscle growth, for example but no limited to a constitutively activated Akt1, under the control of an inducible promoter, for example, an inducible muscle-specific promoter are used for expression analysis, for example gene and/or protein expression analysis.

Another aspect of the invention relates to methods to identify proteins that favorably affect muscle growth, angiogenesis, obesity, insulin sensitivity and cardiovascular function using the assays of the present invention. The present invention relates to comparison of the gene expression profile of cells and/or tissues from transgenic animals and/or transgenic cells, expressing the muscle-related proteins with cells/and tissues where the muscle-related protein is not expressed. In some embodiments secondary gene expression is performed.

In further embodiments, differentially expressed genes are assessed both functionally and characteristically. For example, a functional assessments is, for example their ability to, when expressed, modulate muscle mass and/or modulate angiogenesis, modulate obesity, modulate fat mass, and/or modulate the recruitment of satellite cells and modulate muscle regeneration. As used herein, the term “modulate” refers to an increase or decrease in the metabolic function being assessed. Characteristic assessment is, for example, screening differentially expressed genes for possession of a secretion signal and/or lack of transmembrane domains, or other structural domains.

In some embodiments, the gene expression profile is obtained from any tissue or cell from a transgenic animal of the present invention. In some embodiments, the tissue is muscle tissue. In alternative embodiments, the tissue is fat tissue, liver tissue, cardiac tissue, spleen tissue, neurological tissue and the like.

The muscle secreted factors, also referred to herein as “factors associated with muscle growth” identified by the methods of the present invention may be useful for the treatment of a number of pathological conditions including muscle-wasting diseases, obesity, diabetes, tissue ischemia and bone disease, and muscle degenerative diseases, atrophy and disorders associated with angiogenesis.

In one embodiment, the method of the present invention provides methods for modulating muscle mass in an organism. In one embodiment, the present invention provide methods for increasing muscle mass, the method comprising administrating or delivery of the genes and/or gene products (i.e. proteins), or agonists thereof, identified by the methods of the present invention and characterized to increase muscle growth (muscle hypertrophy) and/or increase muscle regeneration. In an alternative embodiment, the if the muscle secreted protein identified by the methods of the present invention was characterized to decrease muscle mass, then the present invention provides methods to increase muscle mass by administering an agent that functions as an antagonist or inhibitor of such an identified muscle secreted protein.

In yet another embodiment, the present invention relates to methods for modulating glucose and/or insulin insensitivity in an organism. For example, one embodiment the present invention provides methods to increase insulin and glucose sensitivity, the method comprising of administering or delivering the genes and/or gene products (i.e. proteins), or homologues or agonists thereof, identified by the methods of the present invention and characterized to function as a metabolic regulator to increase insulin and/or glucose sensitivity.

In yet another embodiment, the present invention relates to methods for treating obesity in an organism. The method comprises administration or delivery of the genes and/or gene products (i.e. proteins), of functional derivates or agonists thereof, identified by the methods of the present invention and characterized to function to decrease muscle mass and/or fat mass and/or increase VO2 and/or increase fatty acid or administration or agonists thereof.

In yet another embodiment, the present invention relates to methods for modulating angiogenesis in an organism. In one embodiment, one can increase angiogenesis by administering or delivering the genes and/or gene products (i.e. proteins), or functional derivatives or agonists thereof, identified by the methods of the present invention and characterized to increase blood vessel formation and angiogenesis, or administration or agonists thereof. In an alternative embodiment, the present invention relates to methods for decreasing angiogenesis in an organism, the method comprises administration or delivery of an inhibitor and/or antagonist to at least one genes and/or gene products (i.e. proteins) identified by the methods of the present invention and characterized to increase blood vessel formation and angiogenesis.

### Definitions

Unless defined herein, terms used herein have their ordinary meanings, and can be further understood in the context of the specification.

A “transgenic animal” (e.g., a mouse or rat) is an animal having in some or all of its cells a transgene, which transgene was introduced into the animal or an ancestor of the animal at a prenatal, e.g., an embryonic stage. A “transgene” is a DNA that is integrated into the genome of a cell from which a transgenic animal develops.

As used herein, the term “operatively linked,” refers to a functional relationship between two or more nucleic acid (e.g., DNA) segments: for example, a promoter or enhancer is operatively linked to a coding sequence if it stimulates the transcription of the sequence in an appropriate host cell or other expression system. Generally, sequences that are operatively linked are contiguous. However, enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

As used herein, “muscle” or “muscle cell” refers to any cell that contributes to muscle tissue. Myoblasts, satellite cells, myotubes, and myofibril tissues are all included in the term “muscle cells”. Muscle cells may include those within skeletal, cardiac and smooth muscles.

As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only intact antibody molecules but also fragments of antibody molecules retaining antigen binding ability. Such fragments are also well known in the art and are regularly employed both in vitro and in vivo. In particular, as used herein, the term “antibody” means not only intact immunoglobulin molecules of any isotype (IgA, IgG, IgE, IgD, IgM) but also the well-known active fragments F(ab′) (2), Fab, Fv, scFv, Fd, V (H) and V (L). For antibody fragments, see, for example “Immunochemistry in Practice” (Johnstone and Thorpe, eds., 1996; Blackwell Science), p. 69.

As used herein, a “factor associated with muscle growth” refers to a gene or gene product identified by the methods of the present invention. The gene products may be proteins, e.g., secreted proteins, membrane bound proteins, etc. Alternatively, the gene products may be functional RNAs, e.g., microRNAs, ribozymes, etc.

As used herein, a “positive regulator of muscle growth” refers to a gene and/or gene product where its expression results in muscle growth, and lack of its expression results in no muscle growth.

As used herein, a “negative regulator of muscle growth” refers to a gene and/or gene product where its expression results in no muscle growth, and lack of its expression results in muscle growth.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G.” a thymine “T” or a cytosine “C”) or RNA (e.g. an A, a G. an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The term “nucleic acid” also refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. These definitions generally refer to a single-stranded molecule, but in specific embodiments will also encompass an additional strand that is partially, substantially or fully complementary to the single-stranded molecule. Thus, a nucleic acid may encompass a double-stranded molecule or a double-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “is.” The term “gene” refers to the segment of DNA involved in producing a polypeptide chain, it includes regions preceding and following the coding region as well as intervening sequences (introns) between individual coding segments (exons). A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence. The term “enhancer” refers to a cis-acting regulatory sequence involved in the transcriptional activation of a nucleic acid sequence. An enhancer can function in either orientation and may be upstream or downstream of the promoter.

As used herein, the term “gene product(s)” is used to refer to include RNA transcribed from a gene, or a polypeptide encoded by a gene or translated from RNA.

The terms “polypeptide” and “protein” are used interchangeably to refer to a polymer of amino acid residues, and are not limited to a minimum length. Thus, peptides, oligopeptides, dimers, multimers, and the like, whether produced biologically, recombinantly, or synthetically and whether composed of naturally occurring or non-naturally occurring amino acids, are included within the definition. Both full-length proteins and fragments thereof are encompassed by the definition. The terms also include co-translational (e.g., signal peptide cleavage) and post-translational modifications of the polypeptide, such as, for example, dissulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications may be deliberate, as through site-directed mutagenesis, or may be accidental, such as through mutations of hosts that produce the proteins, or errors due to PCR amplification or other recombinant DNA methods. Recombinant, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide with which it is associated in nature. The term recombinant as used with respect to a protein or polypeptide, means a polypeptide produced by expression of a recombinant polynucleotide. The term recombinant as used with respect to a host cell means a host cell into which a recombinant polynucleotide has been introduced.

The term a “dominant negative form” of a molecule, is a structurally altered protein that exerts the opposite phenotypic action on a cell relative to the wild-type protein. For example a dominant negative form of myostatin, is a variant of myostain that is capable of inhibiting normal signaling of that

The term “functional derivative” refers to an entity which possess a biological activity (either functional or structural) that is substantially similar to a biological activity of the entity or molecule its is a functional derivative of. The term functional derivative is intended to include the fragments, variants, analogues or chemical derivatives of a molecule.

The term “functional derivatives” is intended to include the “fragments,” “variants,” “analogs,” or “chemical derivatives” of a molecule. A “fragment” of a molecule, is meant to refer to any polypeptide subset of the molecule. Fragments of, for example a muscle secreted protein, which have the activity and which are soluble (i.e not membrane bound) are also encompassed for use in the present invention. A “variant” of a molecule, for example a muscle secreted is meant to refer to a molecule substantially similar in structure and function to either the entire molecule, or to a fragment thereof. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical. An “analog” of a molecule, for example an analogue of a muscle secreted protein is meant to refer to a molecule substantially similar in function to either the entire molecule or to a fragment thereof As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa. (1990).

The term “entity” refers to any structural molecule or combination of molecules.

The terms “subject” refers to an animal, for example a human, to whom treatment, including prophylactic treatment, with the pharmaceutical composition according to the present invention, is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, amlady, disorder, sickness, illness, complaint, inderdisposion, affection.

An “insulin-resistant disorder” is a disease, condition, or disorder resulting from a failure of the normal metabolic response of peripheral tissues (insensitivity) to the action of exogenous insulin, i.e., it is a condition where the presence of insulin produces a subnormal biological response. In clinical terms, insulin 15 resistance is present when normal or elevated blood glucose levels persist in the face of normal or elevated levels of insulin. It represents, in essence, a glycogen synthesis inhibition, by which either basal or insulin stimulated glycogen synthesis, or both, are reduced below normal levels. Insulin resistance plays a major role in Type 2 diabetes, as demonstrated by the fact that the hyperglycemia present in Type 2 diabetes can sometimes be reversed by diet or weight loss sufficient, apparently, to restore the sensitivity of peripheral 20 tissues to insulin. The term includes abnormal glucose tolerance, as well as the many disorders in which insulin resistance plays a key role, such as obesity, diabetes mellitus, ovarian hyperandrogenism, and hypertension. “Diabetes mellitus” refers to a state of chronic hyperglycemia, i.e., excess sugar in the blood, consequent upon a relative or absolute lack of insulin action. There are three basic types of diabetes mellitus, 25 type I or insulin-dependent diabetes mellitus (IDDM), type II or non-insulin-dependent diabetes mellitus (NIDDM), and type A insulin resistance, although type A is relatively rare. Patients with either type I or type II diabetes can become insensitive to the effects of exogenous insulin through a variety of mechanisms. Type A insulin resistance results from either mutations in the insulin receptor gene or defects in post-receptor sites of action critical for glucose metabolism. Diabetic subjects can be easily recognized by the physician, and are 30 characterized by hyperglycemia, impaired glucose tolerance, glycosylated hemoglobin and, in some instances, ketoacidosis associated with trauma or illness. The term “Non-insulin dependent diabetes mellitus” or “NIDDM” refers to Type II diabetes. NIDDM patients have an abnormally high blood glucose concentration when fasting and delayed cellular uptake of glucose following meals or after a diagnostic test known as the glucose tolerance test. NIDDM is diagnosed based on 35 recognized criteria (American Diabetes Association, Physician's Guide to Insulin-Dependent (Type I) Diabetes, 1988; American Diabetes Association, Physician's Guide to Non-Insulin-Dependent (Type II) Diabetes, 1988).

The term “antagonist” or “inhibitor” are used interchangeably herein, refers to any agent or entity capable of inhibiting or suppressing the expression or activity of a protein, polypeptide portion thereof, or polynucleotide. Thus, the antagonist may operate to prevent transcription, translation, post-transcriptional or post-translational processing or otherwise inhibit the activity of the protein, polypeptide or polynucleotide in any way, via either direct of indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide.

The term “inhibiting” as used herein does not necessarily mean complete inhibition of expression and/or activity. Rather, expression or activity, of the protein, polypeptide or polynucleotide is inhibited to an extent, and/or for a time, sufficient to produce the desired effect.

The term “agonist” refers to any agent or entity capable of activating or enhancing the expression or activity of a protein, polypeptide portion thereof, or polynucleotide. Thus, an agonist may operate to promote gene expression, for example promote gene transcription, translation, post-transcriptional or post-translational processing or otherwise activate the activity of the protein, polypeptide or polynucleotide in any way, via either direct or indirect action. An agonist may for example be a nucleic acid, peptide, or any other suitable chemical compound or molecule or any combination of these. Additionally, it will be understood that in indirectly promoting the activity of a protein, polypeptide of polynucleotide, an agonist may affect the activity of the cellular molecules which may in turn act as regulators or the protein, polypeptide or polynucleotide itself. Similarly, an agonist may affect the activity of molecules which are themselves subject to the regulation or modulation by the protein, polypeptide of polynucleotide. An agonist also refers to any agent that is capable of causing an increase in the activity of a gene and/or gene product in a cell, whether it was present in the cell or absent in the cell prior to adding such an agent. For example, an agent that activates a specific muscle secreted protein is an agent that can activate the expression of the specific muscle secreted protein nucleic acid already present in a cell, or an agent can be a nucleic acid encoding specific muscle secreted protein or a functional derivative thereof, or an agent can be a polypeptide of the specific muscle secreted protein, regardless of whether specific muscle secreted protein is already present in the cell, or an agent can be a specific muscle secreted protein mimetic or functional derivative, for example an analogue of the specific muscle secreted protein.

The term “activating” or “activates” are used interchangeably herein, refers to the general increase in activity of a protein, polypeptide portion thereof, or polynucleotide or a metabolic regulator of the present invention. Activation does not necessarily mean complete activation of expression and/or activity of the metabolic regulator, rather, a general or total increase in the expression or activity of the protein, polypeptide or polynucleotide that is activated to an extent, and/or for a time, sufficient to produce the desired effect.

The term “RNAi” as used herein refers to RNA interference (RNAi) a RNA-based molecule that inhibits gene expression. RNAi refers to a means of selective post-transcriptional gene silencing by destruction of specific mRNA by small interfering RNA molecules (siRNA). The siRNA is typically generated by cleavage of double stranded RNA, where one strand is identical to the message to be inactivated.

The term “shRNA” as used herein refers to short hairpin RNA which functions as RNAi and/or siRNA species but differs in that shRNAi species are double stranded hairpin-like structure for increased stability.

The cells used in the invention can also be cultured cells, e.g. in vitro or ex vivo. For example, cells cultured in vitro in a culture medium. Alternatively, for ex vivo cultured cells, cells can be obtained from a subject, where the subject is healthy and/or affected with a disease. Cells can be obtained, as a non-limiting example, by biopsy or other surgical means know to those skilled in the art. Cells used in the invention can present in a subject, e.g. in vivo. For the invention on use on in vivo cells, the cell is preferably found in a subject and display characteristics of the disease, disorder or malignancy pathology

As used herein, the term “treating” includes reducing or alleviating at least one adverse effect or symptom of a condition, disease or disorder associated with inappropriate proliferation, for example cancer.

As used herein, the terms “administering,” and “introducing” are used interchangeably herein and refer to the placement of the agents targeting metabolic regulators of the present invention into a subject by a method or route which results in at least partial localization of the agents metabolic regulators at a desired site. The compounds of the present invention can be administered by any appropriate route which results in an effective treatment in the subject.

The term “regeneration” means regrowth of a cell population, organ or tissue, and in some embodiments after disease or trauma.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The articles “a” and “an” are used herein to refer to one or to more than one (i.e., to at least one) of the grammatical object of the article. By way of example, “an element” means one element or more than one element.

### Muscle Related Proteins

In one embodiment, the invention provides transgenic animals wherein at least one transgene is incorporated into the nuclear DNA of the animal. The transgene comprises the coding sequence of an AKT gene, e.g., human AKT1, e.g. mouse Akt1, e.g., AKT1 paralogs such as Akt2 or Akt3, operably linked to an inducible promoter, e.g., tetracycline operator sequences, for regulated expression of the transgene. The transgenic animal further comprises a transgene comprising exogenous DNA coding for an inducer of the inducible promoter, e.g., tetracycline transactivator protein, e.g., operably linked to a tissue specific promoter, e.g., a muscle-specific promoter, e.g., muscle creatine kinase (MCK) promoter. Alternatively, the AKT transgene may be operably linked to a tissue-specific promoter.

AKT (also known as PKB or Rac-PK beta), a serine/threonine protein kinase, is the cellular homologue of the product of the v-akt oncogene. AKT comprises an N-terminal pleckstrin homology (PH) domain, a kinase domain and a C-terminal “tail” region. Three isoforms of human AKT kinase (AKT-1, -2 and -3) have been reported so far [(Cheng, J. Q., Proc. Natl. Acad. Sci. USA 1992, 89, 9267-9271); (Brodbeck, D. et al., J. Biol. Chem. 1999, 274, 9133-9136)]. The PH domain binds 3-phosphoinositides, which are synthesized by phosphatidyl inositol 3-kinase (PI3K) upon stimulation by growth factors such as platelet derived growth factor (PDGF), nerve growth factor (NGF) and insulin-like growth factor (IGF-1) [(Kulik et al., Mol. Cell. Biol., 1997, 17, 1595-1606,); (Hemmings, B. A., Science, 1997, 275, 628-630)]. Lipid binding to the PH domain promotes translocation of AKT to the plasma membrane and facilitates phosphorylation by another PH-domain-containing protein kinases, PDK1 at Thr308, Thr309, and Thr305 for the AKT isoforms 1, 2 and 3, respectively. A second, as of yet unknown, kinase is required for the phosphorylation of Ser473, Ser474 or Ser472 in the C-terminal tails of AKT-1, -2 and -3 respectively, in order to yield a fully activated AKT enzyme. Once localized to the membrane, AKT mediates several functions within the cell including the metabolic effects of insulin (Calera, M. R. et al., J. Biol. Chem. 1998, 273, 7201-7204) induction of differentiation and/or proliferation, protein synthesis and stress responses (Alessi, D. R. et al., Curr. Opin. Genet. Dev. 1998, 8,55-62).

The muscle related protein can be, for example, a human or non-human mammalian protein. Suitable muscle related proteins are, for example, Akt 1 proteins, also known under alternative names as; RAC, PRKBA, Akt and PKB. Akt1 can be identified by RefSeq ID NO: NM-005163; NM_{—}001014431; NM_{—}0014432 (SEQ ID NO: 22) and by NP_{—}001014431; NP_{—}001014432; NP_{—}005154 (SEQ ID NO: 23). The muscle related protein also can be a modified muscle related protein, such as, for example, those disclosed in U.S. Patent Publication Nos. 2004/0048255 and 2004/0132156 (the disclosures of which are incorporated by reference herein). Akt nucleic acid and protein sequences are known to one skilled in the art, and homologues, variants and functional derivatives of Akt are also encompassed for use in the methods of the present invention.

In some embodiments, the muscle related transgene is Akt2 (RefSeq No: NM_{—}007434) (SEQ ID NO: 27); Akt3 (RefSeq No: NM_{—}011785) (SEQ ID NO: 28) or PI-3Kinase (RefSeq No: NM-002645) (SEQ ID NO: 25) or homologues or variants or functional derivatives thereof, for example human homologues. In some embodiments, the muscle related transgene is mTor or S6-kinase, or homologues, variants or functional derivatives thereof which are commonly known by person of ordinary skill in the art.

A human muscle related transgene can be, for example, but not limited to Akt1, Akt2, Akt3, PI-3 Kinase, mTor or S6-kinase etc or homologues or variants and functional derivatives thereof. In other embodiments, the muscle related transgene encodes a non-human muscle related protein or a functionally active fragment thereof. The non-human muscle related can be, for example, a mouse, rat, hamster, gerbil, rabbit, bovine, dog, chicken, monkey or other mammalian muscle related. The non-human muscle related transgene can be, for example from mouse.

In some embodiments, the muscle related proteins can be, for example, a negative regulator of muscle growth. An example of such a negative regulator of muscle growth is, for example but not limited to, myostatin, wherein expression of myostatin results in no muscle growth, and lack of myostatin expression results in muscle growth. In such embodiments where the muscle related protein is a negative regulator of muscle growth, the muscle related transgene useful in the present invention is a dominant negative form of the negative regulator of muscle growth, for example a dominant negative form of myostatin and/or a inhibitor of myostatin, for example a nucleic acid inhibitor, for example an RNAi or antisense oligonucleotide of myostatin. In some embodiments, an inhibitor to a negative regulator of muscle growth is a neutralizing antibody, for example but not limited to a neutralizing antibody to myostatin. As used herein, a “dominant negative form” is a variant and/or homologue of a protein that is functionally inactive.

In such embodiments, a muscle related transgene is a negative regulator of muscle growth, for example but not limited to myostatin (NM_{—}005259) SEQ ID NO:26) or homologues or variants thereof, for example a dominant negative form of myostatin.

### Muscle Related Transgenes

The muscle related protein is typically encoded by a muscle related transgene. A “muscle related transgene” refers to a nucleic acid encoding a muscle related protein or a functionally active fragment thereof. The muscle related transgene can be, for example, a portion of genomic DNA, cDNA, mRNA, RNA or a fragment thereof encoding a functional muscle related protein (e.g., a full length muscle related protein) or a functionally active fragment or functional derivative of a muscle related protein. The term “functional derivative” refers to a fragment, homologue, derivative or analog having one or more functions associated with a full-length (wild-type) muscle related polypeptide (e.g., muscle related enzyme activity).

The muscle related transgene can be from the same species as the transgenic animal (e.g., a mouse muscle related transgene overexpressed in a mouse). In some embodiments, the muscle related transgene is a cognate heterologous muscle related transgene. A cognate heterologous muscle related transgene refers to a corresponding gene from another species; thus, if murine muscle related is the reference, human muscle related is a cognate heterologous gene (as is porcine, ovine, or rat muscle related, along with muscle related genes from other species). In some embodiments, the muscle related transgene can encode a human muscle related protein or a functionally active fragment thereof.

The muscle related protein can be a “homologous” or “heterologous polypeptide.” A “heterologous polypeptide,” also referred to as a “xenogenic polypeptide,” is a polypeptide having an amino acid sequence found in an organism not consisting of the transgenic nonhuman animal. As used herein, the term “polypeptide” refers to a polymer of amino acids and its equivalent and does not refer to a specific length of the product; thus, peptides, oligopeptides and proteins are included within the definition of a polypeptide. A derivative is a polypeptide having conservative amino acid substitutions, as compared with another sequence. Derivatives further include other modifications of proteins, including, for example, modifications such as glycosylations, acetylations, phosphorylations, and the like.

A transgene containing various gene segments encoding a cognate heterologous protein sequence may be readily identified, e.g. by hybridization or DNA sequencing, as being from a species of organism other than the transgenic animal. In some embodiments, the cognate muscle related transgene is at least 75%, at least 80%, at least 85%, at least 90% or at least 95% identical to the homologous muscle related transgene. As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection. The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

In some embodiments, the muscle related protein is at least 75%, at least 80%, at least 85%, at least 90% or at least 95% similar to the homologous muscle related protein. As used herein, “similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

The term “substantial similarity” in the context of polypeptide sequences, indicates that the polypeptide comprises a sequence with at least 60% sequence identity to a reference sequence, or 70%, or 80%, or 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ by one or more conservative substitutions.

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

A muscle related transgene also can be identified, for example, by expression of the muscle related transgene from an expression library. (See, e.g., Sambrook et al. (2001). Molecular cloning: a laboratory manual, 3rd ed. (Cold Spring Harbor, N.Y., Cold Spring Harbor Laboratory Press); Ausubel et al., supra.) A mutated endogenous gene sequence can be referred to as a heterologous transgene; for example, a transgene encoding a mutation in a murine muscle related gene which is not known in naturally-occurring murine genomes is a heterologous transgene with respect to murine and non-murine species. The muscle related transgene also can encode a modified muscle related, such as, for example, those disclosed in U.S. Patent Publication Nos. 2004/0048255 and 2004/0132156 (the disclosures of which are incorporated by reference herein).

In some embodiments, the muscle related transgene is expressed from an expression construct comprising a transcriptional unit. A “transcriptional unit” refers to a polynucleotide sequence that comprises a muscle related transgene (e.g., the structural gene (exons)) or a functionally active fragment thereof, a cis-acting linked promoter and other cis-acting sequences necessary for efficient transcription of the structural sequences, distal regulatory elements necessary for appropriate tissue-specific and developmental transcription of the structural sequences (as appropriate), and additional cis sequences for efficient transcription and translation (e.g., polyadenylation site, mRNA stability controlling sequences, or the like). Regulatory or other sequences useful in expression vectors can form part of the transgene sequence. This includes intronic sequences and polyadenylation signals, if not already included. The promoter and other cis-acting sequences are operatively linked to the structural gene.

In some embodiments, the promoter is a tissue-specific promoter, for example a muscle specific promoter. Exemplary muscle-specific promoter are, for example but not limited to, α-myosin heavy chain which is specific for cardiac muscle, the myosin light chain-2 gene control region which is active in skeletal muscle (Shani, Nature 314:283-86 (1985)); and the gonadotropic releasing hormone gene control region which is active in the hypothalamus (Mason et al., Science 234:1372-78 (1986)). In an exemplary embodiment, the promoter is the MKC promoter for expression in skeletal muscle cells. In alternative embodiments, the muscle promoter is a smooth muscle promoter, for example but no limited to SM22a, which directs expression in vascular smooth muscle cells.

In some embodiments, the muscle specific promoter is an inducible muscle-specific promoter. For example, the muscle related transgene, for example, Akt1, e.g., constitutively active isoform of Akt1, may include one or more regulatory elements that provides regulated or conditional expression, e.g., tissue specific or inducible expression, of an operatively linked nucleic acid. One skilled in the art can readily determine an appropriate tissue-specific promoter or enhancer that allows expression of the transgene in a desired tissue. Any of a variety of inducible promoters or enhancers can also be included in the vector for regulatable expression of the transgene. An “inducible” promoter is a system that allows for controllable and careful regulation of gene expression. See, Miller and Whelan, Human Gene Therapy, 8:803-815 (1997). The phrase “inducible promoter” or “inducible system” as used herein includes systems wherein promoter activity can be regulated using an externally delivered agent.

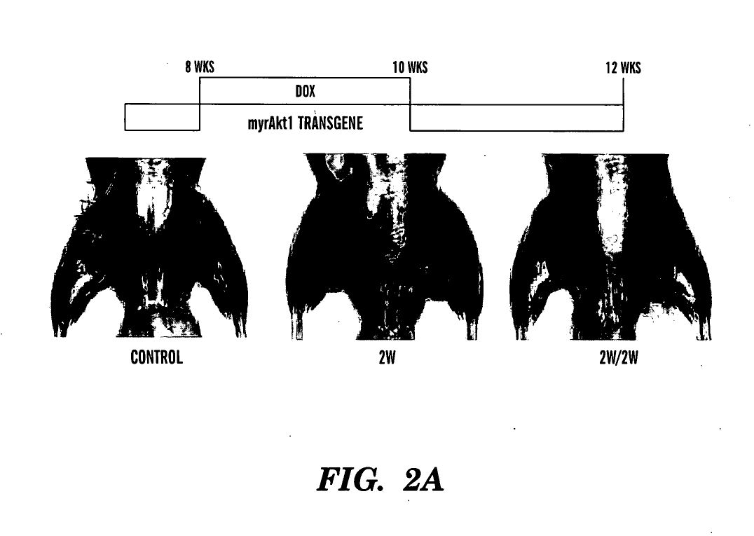

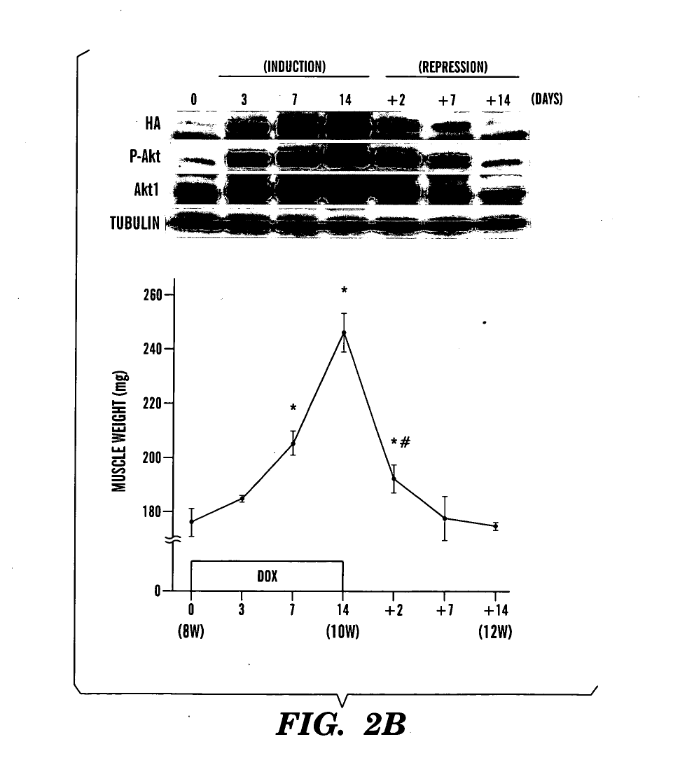

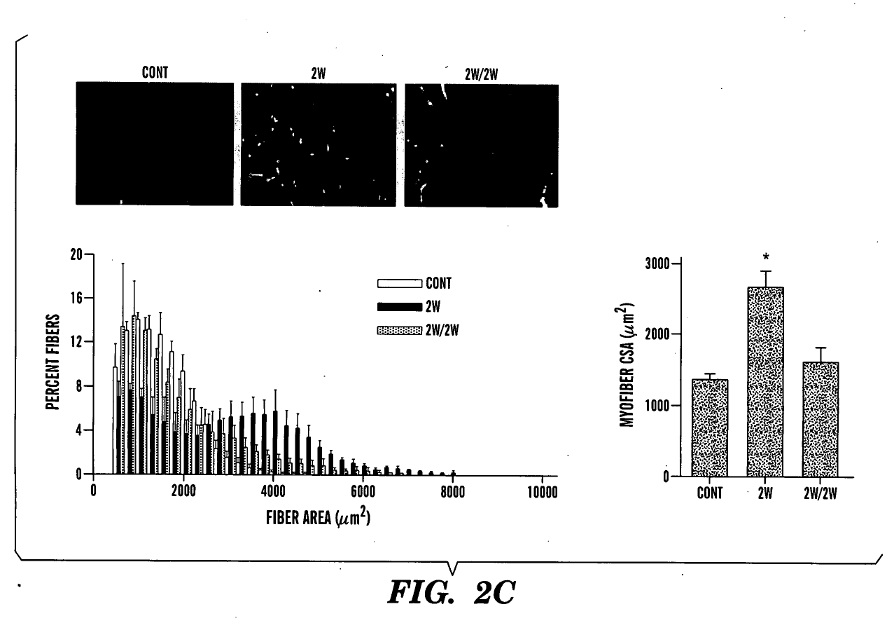

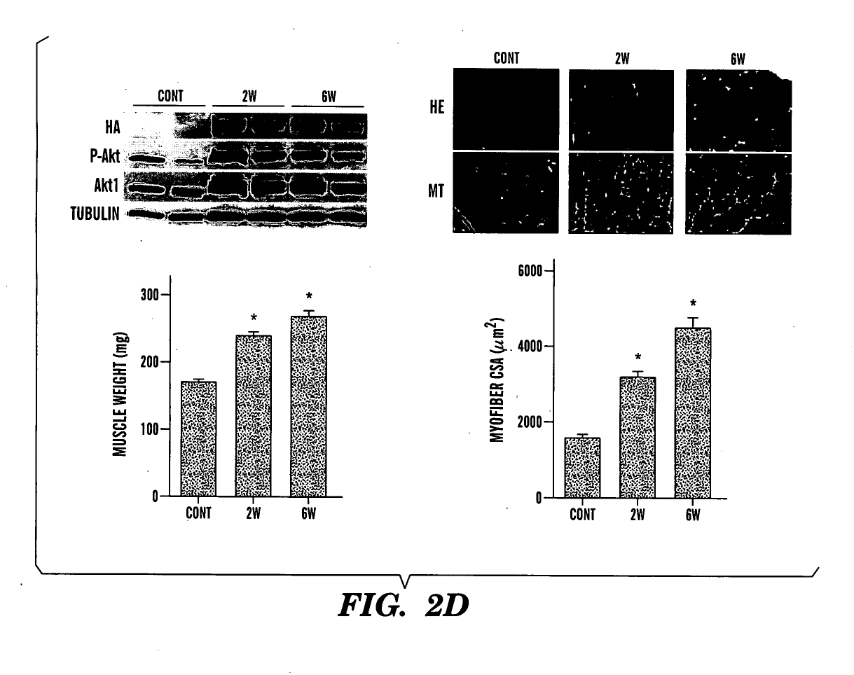

Such systems include, for example, systems using the lac repressor from E. coli as a transcription modulator to regulate transcription from lac operator-bearing mammalian cell promoters (Brown et al. Cell, 49:603-612, 1987); systems using the tetracycline repressor (tetR) (Gossen and Bujard, Proc. Natl. Acad. Sci. USA 89: 5547-5551, 1992; Yao et al., Human Gene Ther. 9:1939-1950, 1998; Shokelt et al., Proc. Natl. Acad. Sci. USA 92:6522-6526, 1995). Other such systems include, for example but not limited to; FK506 dimer, VP16 or p65 using castradiol, RU486/mifepristone, diphenol muristerone or rapamycin (see, Miller and Whelan, supra, at FIG. 2). Yet another example is an ecdysone inducible system (see, e.g. Karns et al, MBC Biotechnology 1:11, 2001). Inducible systems are available, e.g., from Invitrogen, Clontech, and Ariad. Systems using a repressor with the operon are preferred.

Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.); metalothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammary tumor virus (MMTV) induced by steroids such as glucocortocoid and estrogen (Lee et al., Nature, 294:228-232 (1981); and heat shock promoters inducible by temperature changes. Other example systems include a Gal4 fusion inducible by an antiprogestin such as mifepristone in a modified adenovirus vector (Burien et al., Proc. Natl. Acad. Sci. USA, 96:355-360 (1999). Another such inducible system utilizes the drug rapamycin to induce reconstitution of a transcriptional activator containing rapamycin binding domains of FKBP12 and FRAP in an adeno-associated virus vector (Ye et al., Science, 283:88-91 (1999)). Other inducible systems are known by persons skilled in the art and are useful in the methods of the present invention.

Another example of a regulated expression system is the cre/loxP recombinase system of bacteriophage P1. For a description of the cre/loxP recombinase system, see, e.g., Lakso et al. PNAS 89:6232-6236 (1992). Another example of a recombinase system is the FLP recombinase system of S. cerevisiae (O'Gorman. et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

One would adapt these “inducible” systems so that the muscle related protein, for example Akt1, for example constitutively active isoform of is regulated by the addition of a external agent, but is only expressed in muscle cells. Such a promoter system is termed “inducible muscle-specific promoter” herein. Thus, in some embodiments, the muscle related transgene is operatively linked to an inducible promoter that regulates the expression of the muscle regulated transgene in muscle. Such a system may be comprised on one or more exogenous DNAs or transgenes, which may be may be operatively linked. An exemplary system is disclosed in the Examples, for example, the muscle related protein e.g. a constitutively active form of Akt1 is operatively linked to a first promoter, which is an inducible promoter. In such an exemplary system, the transgenic animal may further contain another transgene wherein a second promoter which is muscle-specific promoter and induced on the addition of an exogenous agent regulates the transcription of an inducer of the first promoter. Thus by using such a system, for example, the expression of the muscle related protein, for example Akt1 from the inducible first promoter only occurs when both the additional exogenously added factor and the inducer transcribed from the second promoter is present. Thus, addition of the exogenously added factor enables expression of the muscle related protein under the control of an inducible muscle-specific promoter.

One embodiment of the present invention provides the use of a regulatory element such as a transcriptional regulatory element or an enhancer in the transgene. In one embodiment of the present invention, a “transcriptional regulatory element” or “TRE” is introduced for regulation of the gene of interest. As used herein, a TRE is a polynucleotide sequence, preferably a DNA sequence, that regulates (i.e., controls) transcription of an operably-linked polynucleotide sequence by an RNA polymerase to form RNA. As used herein, a TRE increases transcription of an operably linked polynucleotide sequence in a host cell that allows the TRE to function. The TRE comprises an enhancer element and/or pox promoter element, which may or may not be derived from the same gene. The promoter and enhancer components of a TRE may be in any orientation and/or distance from the coding sequence of interest, and comprise multimers of the foregoing, as long as the desired transcriptional activity is obtained. As discussed herein, a TRE may or may not lack a silencer element. For example,

Another embodiment of the present invention provides an “enhancer” for regulation of the gene of interest. An enhancer is a term well understood in the art and is a polynucleotide sequence derived from a gene which increases transcription of a gene which is operably-linked to a promoter to an extent which is greater than the transcription activation effected by the promoter itself when operably-linked to the gene, i.e. it increases transcription from the promoter. Having “enhancer activity” is a term well understood in the art and means what has been stated, i.e., it increases transcription of a gene which is operably linked to a promoter to an extent which is greater than the increase in transcription effected by the promoter itself when operatively linked to the gene, i.e., it increases transcription from the promoter.

The activity of a regulatory element such as a TRE or an enhancer generally depends upon the presence of transcriptional regulatory factors and/or the absence of transcriptional regulatory inhibitors. Transcriptional activation can be measured in a number of ways known in the art (and described in more detail below), but is generally measured by detection and/or quantization of mRNA or the protein product of the coding sequence under control of (i.e., operatively linked to) the regulatory element. As discussed herein, the regulatory element can be of varying lengths, and of varying sequence composition. By transcriptional activation, it is intended that transcription will be increased above basal levels in the target cell by at least about 2-fold, preferably at least about 5-fold, preferably at least about 10-fold, more preferably at least about 20-fold. More preferably at least about 50-fold, more preferably at least about 100-fold, even more preferably at least about 200-fold, even more preferably at least about 400- to about 500-fold, even more preferably, at least about 1000-fold. Basal levels are generally the level of activity, if any, in a non-target cells, or the level of activity (if any) of a reporter construct lacking the TRE of interest as tested in a target cell type.

The transgene may also include a reporter gene, e.g., reporter genes include β-galactosidase, luciferase, Green fluorescent protein (GFP), chloramphenicol acetyltransferase (CAT), alkaline phosphatase, horse radish peroxidase and the like. In one embodiment, a reporter gene is fused to muscle related gene, for example Akt1, e.g., the constitutively active isoform of Akt1.

Regulatory or other sequences useful in expression vectors can form part of the transgenic sequence. This includes, but is not limited to, intronic sequences, transcription termination signals and polyadenylation signals, if not already included. A conditional regulatory sequence(s) can be operably linked to the transgene to direct expression of the transgene to particular cells.

### Transgenic Animal Model

One aspect of the invention provides methods for the identification of proteins that affect muscle growth angiogenesis, obesity, insulin sensitivity and cardiovascular function using a transgenic animal model as an assay. Accordingly, one aspect of the invention relates to the production and use of a transgenic animal expressing a muscle related protein as disclosed in the section entitled “muscle-related transgenes” above.

A transgenic animal, for example a transgenic non-human animal can be produced which contains selected systems that allow for regulated expression of the muscle related transgene, as discussed in the cre/LoxP recombinase system above.

A transgenic animal, e.g., an invertebrate, such as drosophila, e.g., a vertebrate, such as a mammal, such as a rodent, such as a mouse or a rat, is an animal having cells that contain a transgene, which transgene was introduced into the animal or an ancestor of the animal at a prenatal, e.g., an embryonic stage. A transgene is a DNA, e.g., Akt1, e.g., constitutively active isoform of Akt1, which is integrated into the nuclear genome of a cell from which a transgenic animal develops. The transgene may be integrated into all cells in the animal, including incorporation into the germline of the animal. Alternatively, the animal may be chimeric for the transgene. Methods for generating transgenic animals, particularly animals such as mice or rats, have become conventional in the art and are described, for example, in Ausubel et al. (eds) “Current Protocols in Molecular Biology” John Wiley & Sons, Inc., in Hogan, B., Manipulating the Mouse Embryo, (Cold Spring Harbor Laboratory Press, Cold Spring Harbor, N.Y., 1986) and in U.S. Pat. Nos. 5,614,396 5,487,992, 5,464,764, 5,387,742, 5,347,075, 5,298,422, 5,288,846, 5,221,778, 5,175,384, 5,175,383, 4,873,191, 4,870,009, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, W093/14200, WO 94/06908 and WO 94/28123 also provide information.

The present invention is not limited to a particular animal. A variety of human and non-human animals are contemplated. For example, in some embodiments, rodents (e.g., mice or rats) or primates are provided as animal models for alterations in fat metabolism and screening of compounds.

In other embodiments, the present invention provides commercially useful transgenic animals (e.g., livestock animals such as pigs, cows, or sheep) overexpressing the muscle related protein. It is contemplated that meat from such animals will have desirable properties such as lower fat content and higher muscle content. Any suitable technique for generating transgenic livestock may be utilized. In some preferred embodiments, retroviral vector infection is utilized (See e.g., U.S. Pat. No. 6,080,912 and WO/0030437; each of which is herein incorporated by reference in its entirety).

Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes.

Any techniques known in the art may be used to introduce the transgene, e.g., Akt1, e.g., constitutively active isoform of Akt1, expressibly into animals to produce the mammal lines of animals. Such techniques include, but are not limited to, pronuclear microinjection (U.S. Pat. No. 4,873,191); retrovirus mediated gene transfer into germ lines [Van der Putten, et al., 1985, Proc. Natl. Acad. Sci., USA 82, 6148-6152]; gene targeting in embryonic stem cells, such as homologous recombination mediated gene targeting [Thompson, et al., 1989, Cell 56, 313-321 and U.S. Pat. No. 5,614,396]; electroporation of embryos [Lo, 1983, Mol. Cell. Biol. 3, 1803-1814]; and sperm-mediated gene transfer [Nakanishi and Iritani, Mol. Reprod. Dev. 36:258-261 (1993); Maione, Mol. Reprod. Dev. 59:406 (1998); Lavitrano et al. Transplant. Proc. 29:3508-3509 (1997); Lavitrano et al., Proc. Natl. Acad. Sci. USA 99:14230-5 (2002); Lavitrano et al., Mol. Reprod. Dev. 64-284-91 (2003)). Similar techniques are also described in U.S. Pat. No. 6,376,743; U.S. Pat. Publ. Nos. 20010044937, 20020108132, and 20050229263.

In some embodiments, the muscle related transgene is integrated into the genome of a cell of the transgenic animal. The cell can be a somatic cell or a germline cell. In some embodiments, the muscle related transgene is integrated into the genome of a cell from which a transgenic animal develops and which remains in the genome of the mature animal in one or more cell types or tissues of the transgenic animal.

Muscle related transgenes can be overexpressed in a non-human animal such as a mammal. The mammal can be, for example, a rodent such as a mouse, hamster, guinea pig, rabbit or rat, a primate, a porcine, an ovine, a bovine, a feline, a canine, and the like. In specific embodiments, the transgenic animal can be a sheep, goat, horse, cow, bull, pig, rabbit, guinea pig, hamster, rat, gerbil, mouse, or the like. In other embodiments, the animal can be a bird. In some embodiments, the animal is a chimeric animals (i.e., those composed of a mixture of genetically different cells), a mosaic animals (i.e., an animal composed of two or more cell lines of different genetic origin or chromosomal constitution, both cell lines derived from the same zygote), an immature animal, a fetus, a blastula, and the like.

Transgenic, non-human animals containing a muscle related transgene can be prepared by methods known in the art. In general, a muscle related transgene is introduced into target cells, which are then used to prepare a transgenic animal. A muscle related transgene can be introduced into target cells, such as for example, pluripotent or totipotent cells such as embryonic stem (ES) cells (e.g., murine embryonal stem cells) or other stem cells (e.g., adult stem cells); germ cells (e.g., primordial germ cells, oocytes, eggs, spermatocytes, or sperm cells); fertilized eggs; zygotes; blastomeres; fetal or adult somatic cells (either differentiated or undifferentiated); and the like. In some embodiments, a muscle related transgene is introduced into embryonic stem cells or germ cells of animals (e.g., a rodent) to prepare a transgenic animal overexpressing muscle related.

Methods for generating transgenic animals via embryo manipulation and microinjection, particularly animals such as mice, are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986).

Embryonic stem cells can be manipulated according to published procedures (see, e.g., Teratocarcinomas and Embryonic Stem Cells: A Practical Approach, Robertson (ed.), IRL Press, Washington, D.C. (1987); Zjilstra et al., Nature 342:435-38 (1989); Schwartzberg et al., Science 246:799-803 (1989); U.S. Pat. Nos. 6,194,635; 6,107,543; and 5,994,619; each of which is incorporated herein by reference in their entirety). Methods for isolating primordial germ cells are well known in the art. For example, methods of isolating primordial germ cells from ungulates are disclosed in U.S. Pat. No. 6,194,635 (the disclosure of which is incorporated by reference herein in its entirety).

A muscle related transgene can be introduced into a target cell by any suitable method. For example, a muscle related transgene can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A muscle related transgene can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a muscle related transgene can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999); each incorporated by reference herein in its entirety.)

In some embodiments, a muscle related transgene can be microinjected into pronuclei of fertilized oocytes or the nuclei of ES cells. A typical method is microinjection of the fertilized oocyte. The fertilized oocytes are microinjected with nucleic acids encoding muscle related by standard techniques. The microinjected oocytes are typically cultured in vitro until a “pre-implantation embryo” is obtained. Such a pre-implantation embryo can contain approximately 16 to 150 cells. Methods for culturing fertilized oocytes to the pre-implantation stage include those described by Gordon et al. (Methods in Enzymology 101:414 (1984)); Hogan et al. (in Manipulating the Mouse Embryo, Cold Spring Harbor Laboratory Press, Cold Spring Harbor, N.Y. (1986)); Hammer et al. (Nature 315:680 (1986)); Gandolfi et al. (J. Reprod. Fert. 81:23-28 (1987)); Rexroad et al. (J. Anim. Sci. 66:947-53 (1988)); Eyestone et al. (J. Reprod. Fert. 85:715-20 (1989)); Camous et al. (J. Reprod. Fert. 72:779-85 (1989)); and Heyman et al. (Theriogenology 27:5968 (1989)) for mice, rabbits, pigs, cows, and the like. (These references are incorporated herein in their entirety.) Such pre-implantation embryos can be thereafter transferred to an appropriate (e.g., pseudopregnant) female. Depending upon the stage of development when the muscle related transgene, or a muscle related transgene-containing cell is introduced into the embryo, a chimeric or mosaic animal can result. Mosaic and chimeric animals can be bred to form true germline transgenic animals by selective breeding methods. Alternatively, microinjected or transfected embryonic stem cells can be injected into appropriate blastocysts and then the blastocysts are implanted into the appropriate foster females (e.g., pseudopregnant females).

A muscle related transgene also can be introduced into cells by infection of cells or into cells of a zygote with an infectious virus containing the mutant gene. Suitable viruses include retroviruses (see generally Jaenisch, Proc. Natl. Acad. Sci. USA 73:1260-64 (1976)); defective or attenuated retroviral vectors (see, e.g., U.S. Pat. No. 4,980,286; Miller et al., Meth. Enzymol. 217:581-99 (1993); Boesen et al., Biotherapy 6:291-302 (1994); these references are incorporated herein in their entirety), lentiviral vectors (see, e.g., Naldini et al., Science 272:263-67 (1996), incorporated by reference herein in its entirety), adenoviruses or adeno-associated virus (AAV) (see, e.g., Ali et al., Gene Therapy 1:367-84 (1994); U.S. Pat. Nos. 4,797,368 and 5,139,941; Walsh et al., Proc. Soc. Exp. Biol. Med. 204:289-300 (1993); Grimm et al., Human Gene Therapy 10:2445-50 (1999); the disclosures of which are incorporated by reference herein in their entirety).

Viral vectors can be introduced into, for example, embryonic stem cells, primordial germ cells, oocytes, eggs, spermatocytes, sperm cells, fertilized eggs, zygotes, blastomeres, or any other suitable target cell. In an exemplary embodiment, retroviral vectors which transduce dividing cells (e.g., vectors derived from murine leukemia virus; see, e.g., Miller and Baltimore, Mol. Cell. Biol. 6:2895 (1986)) can be used. The production of a recombinant retroviral vector carrying a gene of interest is typically achieved in two stages. First, a muscle related transgene can be inserted into a retroviral vector which contains the sequences necessary for the efficient expression of the muscle related transgene (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3[prime] regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Oocytes which have not undergone the final stages of gametogenesis are typically infected with the retroviral vector. The injected oocytes are then permitted to complete maturation with the accompanying meiotic divisions. The breakdown of the nuclear envelope during meiosis permits the integration of the proviral form of the retrovirus vector into the genome of the oocyte. When pre-maturation oocytes are used, the injected oocytes are then cultured in vitro under conditions that permit maturation of the oocyte prior to fertilization in vitro. Oocytes can be matured in vivo and employed in place of oocytes matured in vitro. Methods for the superovulation and collection of in vivo matured (e.g., oocytes at the metaphase 2 stage) oocytes are known for a variety of mammals (e.g., for superovulation of mice, see Hogan et al., in Manipulating the Mouse Embryo: A Laboratory Manual, 2nd ed., Cold Spring Harbor Laboratory Press, Cold Spring Harbor, N.Y. (1994), pp. 130-133; the disclosure of which is incorporated by reference herein in its entirety).

In some embodiments, a transgenic animal is prepared by nuclear transfer. The terms “nuclear transfer” or “nuclear transplantation” refer to methods of preparing transgenic animals wherein the nucleus from a donor cell is transplanted into an enucleated oocyte. Nuclear transfer techniques or nuclear transplantation techniques are known in the art. (See, e.g., Campbell et al., Theriogenology 43:181 (1995); Collas and Barnes, Mol. Reprod. Dev. 38:264-67 (1994); Keefer et al., Biol. Reprod. 50:935-39 (1994); Sims et al., Proc. Natl. Acad. Sci. USA 90:6143-47 (1993); Prather et al., Biol. Reprod. 37:59-86 (1988); Roble et al., J. Anim. Sci. 64:642-64 (1987); International Patent Publications WO 90/03432, WO 94/24274, and WO 94/26884; U.S. Pat. Nos. 4,994,384 and 5,057,420; the disclosures of which are incorporated by reference herein in their entirety.) For example, nuclei of transgenic embryos, pluripotent cells, totipotent cells, embryonic stem cells, germ cells, fetal cells or adult cells (i.e., containing a muscle related transgene) can be transplanted into enucleated oocytes, each of which is thereafter cultured to the blastocyst stage. (As used herein, the term “enucleated” refers to cells from which the nucleus has been removed as well as to cells in which the nucleus has been rendered functionally inactive.) The nucleus containing a muscle related transgene can be introduced into these cells by any suitable method. The transgenic cell is then typically cultured in vitro to the form a pre-implantation embryo, which can be implanted in a suitable female (e.g., a pseudo-pregnant female).

The transgenic embryos optionally can be subjected, or resubjected, to another round of nuclear transplantation. Additional rounds of nuclear transplantation cloning can be useful, when the original transferred nucleus is from an adult cell (e.g., fibroblasts or other highly or terminally differentiated cell) to produce healthy transgenic animals.

Other methods for producing a transgenic animal expressing a muscle related transgene include the use male sperm cells to carry the muscle related transgene to an egg. In one example, a muscle related transgene can be administered to a male animal's testis in vivo by direct delivery. The muscle related transgene can be introduced into the seminiferous tubules, into the rete testis, into the vas efferens or vasa efferentia, using, for example, a micropipette.

In some embodiments, a muscle related transgene can be introduced ex vivo into the genome of male germ cells. A number of known gene delivery methods can be used for the uptake of nucleic acid sequences into the cell. Suitable methods for introducing a muscle related transgene into male germ cells include, for example, liposomes, retroviral vectors, adenoviral vectors, adenovirus-enhanced gene delivery systems, or combinations thereof.

Following transfer of a muscle related transgene into male germ cells, a transgenic zygote can be formed by breeding the male animal with a female animal. The transgenic zygote can be formed, for example, by natural mating (e.g., copulation by the male and female vertebrates of the same species), or by in vitro or in vivo artificial means. Suitable artificial means include, but are not limited to, artificial insemination, in vitro fertilization (IVF) and/or other artificial reproductive technologies, such as intracytoplasmic sperm injection (ICSI), subzonal insemination (SUZI), partial zona dissection (PZD), and the like, as will be appreciated by the skilled artisan. (See, e.g., International Patent Publication WO 00/09674, the disclosure of which is incorporated by reference herein in its entirety.)

A variety of methods can be used to detect the presence of a muscle related transgene in target cells and/or transgenic animals. Since the frequency of transgene incorporation can be low, although reliable, the detection of transgene integration in the pre-implantation embryo can be desirable. In one aspect, embryos are screened to permit the identification of muscle related-transgene-containing embryos for implantation to form transgenic animals. For example, one or more cells are removed from the pre-implantation embryo. When equal division of the embryo is used, the embryo is typically not cultivated past the morula stage (32 cells). Division of the pre-implantation embryo (see, e.g., Williams et al., Theriogenology 22:521-31 (1986)) results in two “hemi-embryos” (hemi-morula or hemi-blastocyst), one of which is capable of subsequent development after implantation into the appropriate female to develop in utero to term. Although equal division of the pre-implantation embryo is typical, it is to be understood that such an embryo can be unequally divided either intentionally or unintentionally into two hemi-embryos. Essentially, one of the embryos which is not analyzed usually has a sufficient cell number to develop to full term in utero. In a specific embodiment, the hemi-embryo (which is not analyzed), if shown to be transgenic, can be used to generate a clonal population of transgenic animals, such as by embryo splitting.

One of the hemi-embryos formed by division of pre-implantation embryos can be analyzed to determine if the muscle related transgene has integrated into the genome of the organism. Each of the other hemi-embryos can be maintained for subsequent implantation into a recipient female, typically of the same species. A typical method for detecting a muscle related transgene at this early stage in the embryo's development uses these hemi-embryos in connection with allele-specific PCR, which can differentiate between a muscle related transgene and an endogenous transgene. (See, e.g., McPherson et al. (eds) PCR2: A Practical Approach, Oxford University Press (1995); Cha et al., PCR Methods Appl. 2:14-20 (1992); the disclosures of which are incorporated by reference herein.)

After a hemi-embryo is identified as a transgenic hemi-embryo, it optionally can be cloned. Such embryo cloning can be performed by several different approaches. In one cloning method, the transgenic hemi-embryo can be cultured in the same or in a similar media as used to culture individual oocytes to the pre-implantation stage. The “transgenic embryo” so formed (typically a transgenic morula) can then be divided into “transgenic hemi-embryos” which can be implanted into a recipient female to form a clonal population of two transgenic non-human animals. Alternatively, the two transgenic hemi-embryos obtained can be again cultivated to the pre-implantation stage, divided, and recultivated to the transgenic embryo stage. This procedure can be repeated until the desired number of clonal transgenic embryos having the same genotype are obtained. Such transgenic embryos can then be implanted into recipient females to produce a clonal population of transgenic non-human animals.

In addition to the foregoing methods for detecting the presence of a muscle related transgene, other methods can be used. Such methods include, for example, in utero and postpartum analysis of tissue. In utero analysis can be performed by several techniques. In one example, transvaginal puncture of the amniotic cavity is performed under echoscopic guidance (see, e.g., Bowgso et al., Bet. Res. 96:124-27 (1975); Rumsey et al., J. Anim. Sci. 39:386-91 (1974)). This involves recovering amniotic fluid during gestation. Most of the cells in the amniotic fluid are dead. Such cells, however, contain genomic DNA which can be subjected to analysis (e.g., by PCR) for the muscle related transgene as an indication of a successful transgenesis. Alternatively, fetal cells can be recovered by chorion puncture. This method also can be performed transvaginally and under echoscopic guidance. In this method, a needle can be used to puncture the recipient animal's placenta, particularly the placentonal structures, which are fixed against the vaginal wall. Chorion cells, if necessary, can be separated from maternal tissue and subjected to PCR analysis for the muscle related transgene as an indication of successful transgenesis.

The presence of a muscle related transgene also can be detected after birth. In such cases, the presence of a muscle related transgene can be detected by taking an appropriate tissue biopsy, such as from an ear or tail of the putative transgenic animal. The presence of a muscle related transgene can also be detected by assaying for expression of the muscle related transgene polypeptide in a tissue.

The location and number of integration events can be determined by methods known to the skilled artisan. (See, e.g., Ausubel et al., supra; Sambrook et al., supra.) For example, PCR or Southern blot analysis of genomic DNA extracted from infected oocytes and/or the resulting embryos, offspring and tissues derived therefrom, can be employed when information concerning the site of integration of the viral DNA into the host genome is desired. To examine the number of integration sites present in the host genome, the extracted genomic DNA can typically be digested with a restriction enzyme which cuts at least once within the vector sequences. If the enzyme chosen cuts twice within the vector sequences, a band of known (i.e., predictable) size is generated in addition to two fragments of novel length which can be detected using appropriate probes.

Clones of the non-human transgenic animals described herein can also be produced according to the methods described in Wilmut et al., Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring born of this female foster animal will be a clone of the animal from which the cell, e.g., the somatic cell, is isolated.

Other methods of preparing transgenic animals are disclosed, for example, in U.S. Pat. Nos. 5,633,076 or 6,080,912; and in International Patent Publications WO 97/47739, WO 99/37143, WO 00/75300, WO 00/56932, and WO 00/08132, the disclosures of which are incorporated herein by reference in their entirety.

A transgenic animal containing a muscle related transgene can used as a founder animal breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In a related aspect, a non-human transgenic animal overexpressing a muscle related transgene can be a source of cells to establish cell lines expressing or overexpressing the muscle related transgene. For example, cell lines can be derived from mice that overexpress a mouse or cognate, heterologous muscle related transgene.

In an alternative embodiment, a transgenic mouse model of the present invention can include administration of inhibitors of negative regulators of muscle growth. For example a transgenic animal model of the present invention is achieved by administering to an animal (not necessarily a transgenic animal) a inhibitor to a muscle related protein wherein the muscle related protein is a negative regulator of muscle growth. As an exemplary example, but not limited to, a transgenic model of the present invention can be administering an effective amount of inhibitory antibody to myostatin to the animal, wherein the inhibitory antibody reduces the expression and/or activity of myostatin resulting in muscle growth.

The transgenic animals of the invention can have other genetic alterations in addition to the presence of the muscle related transgenes. For example, the host's genome may be altered to affect the function of endogenous genes encoding muscle related proteins (e.g., endogenous Akt or Tpl2), contain marker genes, or other genetic alterations consistent with the goals of the present invention. For example, although not necessary to the operability of the invention, the transgenic animals described herein may have alterations to endogenous genes in addition to (or alternatively for Akt), the genetic alterations described above. For example, the host animals may be knockouts for myostatin as is consistent with the goals of the invention.

Clones of the transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring born of this female foster animal will be a clone of the animal from which the cell, e.g., the somatic cell, is isolated.

The invention also includes a population of cells isolated from the transgenic animal of the invention. For example, the transgenic animals of the invention can be used as a source of cells for cell culture.

### Cell-Based Assay

Another aspect of the invention provides methods for the identification of genes and/or gene products (i.e proteins) that affect muscle growth angiogenesis, obesity, insulin sensitivity and cardiovascular function using a cell-based assay. Accordingly, the present invention provides methods for the generation and production of such a cell-based assay. In one embodiment, the cell-based assay comprises cells expressing a muscle related protein as disclosed in the section entitled “muscle related transgenes” above.

In some embodiments, the muscle related protein is Akt. In some embodiments, the Akt is constitutively active form of Akt. In other embodiments, the Akt is Akt1, Akt2, Akt3 or homologues or variants thereof. In alternative embodiments, the muscle related protein is PI-3 kinase or homologues or variants thereof, or mTOR or S6-kinase or homologues or variants or fragments thereof.

In some embodiments, the cell expressing a muscle related protein is a muscle cells, for example a myogenic cell. In some embodiments, the muscle cell is a skeletal muscle cell. In some embodiments the cell is from a cell line, and in some embodiments, the cell line is a myogenic cell line for example but not limited to C2C12 cells. In alternative embodiments, the cell is a mouse or human cell. In alternative embodiments, the muscle cell is a primary muscle cells, for example a human primary muscle cell or an animal muscle cell.

In some embodiments, the cells are obtained from transgenic animals of the present invention. In some embodiments, the cells are, for example but not limited to, liver cells, adipose cells, muscle cells, smooth muscle cells, skeletal muscle cells, cardiac muscle cells, vascular endothelial cells, pancreatic cells, for example pancreatic β-cells and the like.

In other embodiments, the cell is a primary cell obtained from the transgenic animal of the present invention, or a cell line obtained from a transgenic animal model of the present invention, for example but not limited to a transgenic mouse model expressing the muscle related protein under an inducible and/or tissue specific promoter, for example a transgenic mouse expressing a constitutively active isoform of Akt1 under an inducible promoter, or a transgeneic animal expressing Akt2, Akt3, PI-3K, mTOR or S6-kinase or homologues, variants or functional derivatives thereof.

Methods to introduce the muscle related transgene into the cell are well known in the art, and are any suitable method as disclosed in the section entitled “transgenic mouse models” can be used. Such methods include for example, introduction of a muscle related transgene into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A muscle related transgene can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a muscle related transgene can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999); each incorporated by reference herein in its entirety.)

Uses of the Transgenic Animal Models and/or Cell Models of the Invention

In one embodiments, the transgenic animals of the present invention expressing a muscle related transgene, for example but not limited to Akt1, e.g., constitutively active isoform of Akt1, of the present invention may be bred into animals of varying genetic backgrounds, including animals with phenotypes of interest, e.g., obesity, diabetes, angiogenic defects, cardiovascular defects. Such animals are known to the skilled artisan, and, for example, can be found in the Mouse Genome Database (Blake J A, Richardson J E, Bult C J, Kadin J A, Eppig J T, and the members of the Mouse Genome Database Group. 2003. MGD: The Mouse Genome Database. Nucleic Acids Res 31: 193-195; Eppig J T, Blake, J A, Burkhart D L, Goldsmith C W, Lutz C M, Smith C L. 2002. Corralling conditional mutations: a unified resource for mouse phenotypes. Genesis 32:63-65) or the Oak Ridge National Laboratory mutant mouse database.

In alternative embodiments, cells and/or the transgenic animals of the present invention expressing a muscle related transgene, for example but not limited to Akt1, e.g., constitutively active isoform of Akt1 are used in an assay to identify proteins that affect muscle growth, angiogenesis, obesity, insulin sensitivity and/or cardiovascular function. In alternative embodiments, the cells and/or the transgenic animals of the present invention express a muscle related transgene, wherein the muscle related transgene encodes an inhibitor of a muscle related protein, where the muscle related protein is a negative inhibitor of muscle growth, for example, the muscle related protein is myostatin.

The present invention also provides methods for identifying genes and gene products regulated by the muscle related protein, for example Akt1, associated with muscle growth, obesity, insulin sensitivity and cardiovascular function include identification of mRNAs, functional RNAs, e.g., microRNAs, and/or proteins differentially expressed in transgenic animal of the present invention, for example in Akt1-induced transgenic tissue and/or cells in the cell-based assay. Such methods are known to the skilled artisan and may include methods described below.

Genes and gene products identified by the methods of the present invention are useful for further characterization in order to examine the effects of the identified genes and gene products on muscle growth and biology, as well as angiogenesis, insulin sensitivity and fat mass reduction/growth.

The genes identified by the methods of the present invention may be used for the construction of transgenic animals, e.g., knock-out animals, e.g., animals with exogenous expression, for the identification of muscle secreted factors or the study of muscle growth, angiogenesis, obesity, insulin sensitivity and cardiovascular function. Furthermore, transgenic, including knock-out, animals may be bred to the Akt1 transgenic mouse of the present invention.

The genes identified by the methods of the present invention may be analyzed computationally or experimentally for characteristics of interest. In one embodiment, the genes identified as associated with Akt1 expression are computationally screened for characteristics identifying the genes as secreted factors, i.e., possession of putative signal sequences and lack of putative transmembrane domains. Any other domain or sequence characteristics of interest, e.g., nucleic acid or amino acid sequence, may be utilized in selecting genes and gene products from the genes and gene products identified by the methods of the present invention.

Transgenic animal models and/or cells expressing a muscle related transgene can also be used to assay test compounds (e.g., a drug candidate) for efficacy on muscle development, muscle growth, obesity, insulin sensitivity and cardiovascular function in test animals, or in samples or specimens (e.g., a biopsy) from the test animals. In some cases, it will be advantageous to measure the markers of muscle growth, obesity, insulin sensitivity and cardiovascular function in samples, blood, which may be obtained from the test animal without sacrifice of the animal.

Assays for Identifying Proteins that Affect Muscle Growth, Angiogenesis, Obesity, Insulin Sensitivity and/or Cardiovascular Function.

One aspect of the present invention relates to methods to identify proteins that affect muscle growth, angiogenesis, obesity, insulin sensitivity, body weight and/or cardiovascular function. In one embodiment, the methods relate to the use of transgenic animals produced by the methods of the present invention. In another embodiment, the methods relate to the use of a cell-based assay produced by the methods of the present invention. In both embodiments, the methods of the present invention provide for comparative analysis of biological samples, e.g., cells and/or tissue derived from the transgenic animal and/or cell-based assay expressing the muscle related transgene with biological samples, e.g. cells and/or tissue derived from non-transgenic animals and/or cells not comprising and/or not expressing the muscle related transgene.

The cells and/or tissue may be utilized as extracted, e.g., as a tissue lysate, or may be enriched for a particular cell type of interest. The biological sample may include cell culture derived from the transgenic animals of the present invention.

In some embodiments, the tissue is any tissue or cells from the transgenic animal comprising the muscle related transgene. In some embodiments, the tissue is muscle, and in some embodiments the muscle is skeletal muscle. In alternative embodiments, the tissue is liver, adipose and other tissues. In some embodiments, the tissue comprises only one type of tissue, and in alternative embodiments, the tissue comprises a mixture of different tissues.

Dissociation of muscle usually includes digestion with a suitable protease, e.g. collagenase, dispase, etc., followed by trituration until dissociated into myofiber fragments. Fragments are then washed and further enzymatically dissociated to generate a population of myofiber associated cells. An appropriate solution is used for dispersion or suspension. Such solution will generally be a balanced salt solution, e.g. normal saline, PBS, Hanks balanced salt solution, etc., supplemented with fetal calf serum or other naturally occurring factors, in conjunction with an acceptable buffer at low concentration, generally from 5-25 mM. Convenient buffers include HEPES, phosphate buffers, lactate buffers, etc.

In some embodiments, the cells either from the transgenic animal or the cell-based assay are sorted according to a desired cell population. Separation of the desired cell population is achieved by affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, 7-AAD). Any technique may be employed which is not unduly detrimental to the viability of the selected cells.

### Identification of Differentially Expressed Gene Transcripts

In one aspect of the invention, methods to identify proteins that affect muscle growth, angiogenesis, obesity, insulin sensitivity and/or cardiovascular function is done by analysis of differentially expressed gene transcripts between cells and/or tissues from transgenic animals and/or cells from the cell-based assay comprising muscle related transcripts of the present invention compared with cells and/or tissues from animals or cells without muscle related transcripts of the present invention Of particular interest is the examination of gene expression in transgenic animals of the present invention, e.g., in muscle tissue derived from the transgenic animals. The expressed set of genes may be compared between, for example, induced and non-induced tissue, between transgenic and non-transgenic, between transgenics on different genetic backgrounds, between transgenics with differing additional transgenic genes and/or different muscle related transgenes inserted or disrupted. For example, comparison of the gene expression profile of tissue derived from transgenic animals comprising an Akt1 muscle related transgene with that of tissue derived from transgenic animals with a mutated version of Akt1, and/or Akt2 as the muscle related transgene.

In some embodiments, the expression profile of cells from the cell-based assay of the present invention (i.e cells comprising the muscle related transgene, for example cells expressing the activated isoform of the Akt1 transgene) can be compared with the expression profile of cells and/or tissue derived from a transgenic animal of the present invention (i.e cells derived from an transgenic animal expressing the muscle-related transgene, for example a transgenic animal expressing the activated isoform of the Akt1 transgene).

In alternative embodiments, the expressed set of genes (also known as the “expression profile”) may be compared against other subsets of cells, against stem or progenitor cells, against fetal muscle tissue, against adult muscle tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+mRNA. One of skill in the art can readily use these methods to determine differences in the size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

Further PCR-based techniques include, for example, cDNA subtraction; differential display (Liang and Pardee, Science 257:967-971 (1992)); amplified fragment length polymorphism (iAFLP) (Kawamoto et al., Genome Res. 12:1305-1312 (1999)); BeadArray™. technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the methods of the invention is described below in more detail. Hybridization analysis according to the invention can also be carried out using a Micro-Electro-Mechanical System (MEMS), such as the Protiveris' multicantilever array.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

### Identification of Differentially Expressed Proteins

Of further interest is the examination of protein expression in cell-based assay and/or transgenic animals of the present invention, e.g.,examination of the proteins expressed in the cells on induction (transiently or constitutively) of the muscle related transgene and/or examination or proteins expressed in tissue, for example, muscle, derived from the transgenic animals on induction of the muscle related transgene. Protein expression may be compared between, for example induced and non-induced tissue, between transgenic and non-transgenic, between transgenics on different genetic backgrounds, between transgenics with differing additional transgenic genes inserted or disrupted.

In some embodiments, the protein expression of cells from the cell-based assay of the present invention (i.e cells comprising the muscle related transgene, for example cells expressing the activated isoform of the Aka transgene) can be compared with the protein expression of cells and/or tissue derived from a transgenic animal of the present invention (i.e cells derived from an transgenic animal expressing the muscle-related transgene, for example a transgenic animal expressing the activated isoform of the Akt1 transgene).

In alternative embodiments, the expressed set of proteins may be compared against other subsets of cells, for example, against stem or progenitor cells, against fetal muscle tissue, against adult muscle tissue, and the like, as known in the art. Any suitable qualitative or quantitative methods known in the art for detecting specific proteins, including protein signatures, can be used. Differential expression of proteins can be detected by, for example, using protein microarrays, e.g., Plexigen, Inc., Cary N.C. One of skill in the art can readily use these methods to determine differences in the size or amount of proteins between two samples. Also included is the examination of proteins that bind to Akt1 or to other proteins identified by the methods of the present invention using the transgenic animals of the present invention. Methods to identify protein binding partners are well known to the skilled artisan. Such methods include, for example, yeast two-hybrid systems.

The present invention employs methods of separating proteins and for comparing protein expression profiles. Methods of separating proteins are well known to those of skill in the art and include, but are not limited to, various kinds of chromatography (e.g., anion exchange chromatography, affinity chromatography, sequential extraction, and high performance liquid chromatography), and mass spectrometry.

Two-Dimensional Electrophoresis

In one embodiment the present invention employs two-dimensional gel electrophoresis to separate proteins from a biological sample, e.g., a tissue sample derived from a transgenic animal of the present invention, into a two-dimensional array of protein spots.

Two-dimensional electrophoresis is a useful technique for separating complex mixtures of molecules, often providing a much higher resolving power than that obtainable in one-dimension separations. Two-dimensional gel electrophoresis can be performed using methods known in the art (See, e.g., U.S. Pat. Nos. 5,534,121 and 6,398,933). Typically, proteins in a sample are separated first by isoelectric focusing, during which proteins in a sample are separated in a pH gradient until they reach a spot where their net charge is zero (i.e., isoelectric point). This first separation step results in a one-dimensional array of proteins. The proteins in the one-dimensional array are further separated using a technique generally distinct from that used in the first separation step. For example, in the second dimension proteins may be further separated by polyacrylamide gel electrophoresis in the presence of sodium dodecyl sulfate (SDS-PAGE). SDS-PAGE allows further separation based on the molecular mass of the protein.

Proteins in the two-dimensional array can be detected using any suitable methods known in the art. Staining of proteins can be accomplished with calorimetric dyes (e.g., coomassie), silver staining, or fluorescent staining (Ruby Red; SyproRuby). As is known to one of ordinary skill in the art, spots or protein patterns generated can be further analyzed. For example, proteins can be excised from the gel and analyzed by mass spectrometry. Alternatively, the proteins can be transferred to an inert membrane by applying an electric field and the spot on the membrane that approximately corresponds to the molecular weight of a marker can be analyzed by mass spectrometry.

Mass Spectrometry

In certain embodiments the present invention employs mass spectrometry. Mass spectrometry provides a means of “weighing” individual molecules by ionizing the molecules in vacuum and making them “fly” by volatilization. Under the influence of combinations of electric and magnetic fields, the ions follow trajectories depending on their individual mass (m) and charge (z). The “time of flight” of the ion before detection by an electrode is a measure of the mass-to-charge ratio (m/z) of the ion. Mass spectrometry (MS), because of its extreme selectivity and sensitivity, has become a powerful tool for the quantification of a broad range of bioanalytes including pharmaceuticals, metabolites, peptides and proteins.

Matrix-assisted laser desorption ionization-time of flight mass spectrometry (MALDI-TOF MS) is a type of mass spectrometry in which the analyte substance is distributed in a matrix before laser desorption. MALDI-TOF MS has become a widespread analytical tool for peptides, proteins and most other biomolecules (oligonucleotides, carbohydrates, natural products, and lipids). In combination with 2D-gel electrophoresis, MALDI-TOF MS is particularly suitable for the identification of protein spots by peptide mass fingerprinting or microsequencing.

In MALDI-TOF analysis, the analyte is first co-crystallized with a matrix compound, after which pulse UV laser radiation of this analyte-matrix mixture results in the vaporization of the matrix which carries the analyte with it. The matrix therefore plays a key role by strongly absorbing the laser light energy and causing, indirectly, the analyte to vaporize. The matrix also serves as a proton donor and receptor, acting to ionize the analyte in both positive and negative ionization modes. A protein can often be unambiguously identified by MALDI-TOF analysis of its constituent peptides (produced by either chemical or enzymatic treatment of the sample).

Another type of mass spectrometry is surface-enhanced laser desorption ionization-time of flight mass spectrometry (SELDI-TOF MS). Whole proteins can be analyzed by SELDI-TOF MS, which is a variant of MALDI-TOF MS. In SELDI-TOF MS, fractionation based on protein affinity properties is used to reduce sample complexity. For example, hydrophobic, hydrophilic, anion exchange, cation exchange, and immobilized-metal affinity surfaces can be used to fractionate a sample. The proteins that selectively bind to a surface are then irradiated with a laser. The laser desorbs the adherent proteins, causing them to be launched as ions. The SELDI-TOF MS approach to protein analysis has been implemented commercially (e.g., Ciphergen).

Tandem mass spectrometry (MS/MS) is another type of mass spectrometry known in the art. With MS/MS analysis ions separated according to their m/z value in the first stage analyzer are selected for fragmentation and the fragments are then analyzed in a second analyzer. Those of skill in the art will be familiar with protein analysis using MS/MS, including QTOF, Ion Trap, and FTMS/MS. MS/MS can also be used in conjunction with liquid chromatography via electrospray or nanospray interface or a MALDI interface, such as LCMS/MS, LCLCMS/MS, or CEMS/MS.

Other Methods of Protein Analysis

In addition to the methods described above, other methods of protein separation and analysis known in the art may be used in the practice of the present invention. The methods of protein of protein separation and analysis may be used alone or in combination.

Of particular interest are various forms of chromatography. Chromatography is used to separate organic compounds on the basis of their charge, size, shape, and solubilities. Chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography that may be used include: adsorption, partition, affinity, gel filtration, and molecular sieve, and many specialized techniques for using them including column, paper, thin-layer, and gas chromatography (Freifelder, 1982).

Analysis of Protein Markers and Patterns

Following separation of the proteins, the protein markers and protein patterns may be further analyzed. Where, for example, the protein markers have been separated by two-dimensional gel electrophoresis, the protein markers may be visualized by staining the gel. Protein standards having known molecular weights and isoelectric focusing points can be used as landmarks. Gels are preferably stained by Spyro Ruby fluorescent dye. Other dyes, such as silver staining and coomassie blue, are known in the art and could be used.

Gel images may be compared visually and/or electronically. To compare gel images electronically, the gels are first scanned (e.g., Molecular Imager FX (Bio-Rad Laboratories)) and then analyzed using software such as PDQUEST (Bio-Rad Laboratories). Analysis includes spot normalization, spot detection, and comparisons of protein patterns. Spot density may be quantitatively normalized based on the density of each spot versus the total density of all detected spots. The image analysis software may be set up for the analysis of PPM for each spot and also for highlighting fold differences between spots in any set of image comparisons.

In one aspect of the invention, the gel images are compared between biological samples derived from induced transgenic animals and derived from non-induced transgenic animals to identify protein markers and protein patterns that differ between the two.

Following differential expression analysis, spots of interest can be excised from the gel for identification. Those of skill in the art will be familiar with methods, such as mass fingerprinting analysis and microsequencing, which may be used to identify the protein spots. In a preferred embodiment, the ProteomeWorks robotic spot cutter (Bio-Rad Laboratories) is used to excise the spots from the gel. Excised spots are then in-gel digested on a MultiPROBE II (Packard, Downers Grove, Ill.). The gel is then re-hydrated and the digested peptides are extracted from the gel.

Mass spectral analyses of the digested peptides can be performed to identify the protein markers. Those of skill in the art are familiar with mass spectral analysis of digested peptides. In a preferred embodiment, mass spectral analysis is conducted on MALDI-TOF Voyager DE PRO (Applied Biosystems). Spectra should be carefully scrutinized for acceptable signal-to-noise ratio (S/N) to eliminate spurious artifact peaks from the peptide molecular weight lists. Both internal and external standards may be employed. The internal or external standards are considered for calibration of any shift in mass values during mass spectroscopic analysis. External standards are a set proteins of known molecular weight and known m/z value in the mass spectrum. A mixture of external standards is placed on the mass spec chip well next to the well that includes a desired sample. Internal standards are characteristic peaks in the sample spectrum that belong to peptides of the proteolytic enzyme (e.g., trypsin) used to digest protein spots and extracted along with the digested peptides. Those peaks are used for internal calibration of any deviation of spectral peaks of the sample.

Corrected molecular weight lists can then be subjected to database searches (e.g., NCBI and Swiss Protein data banks). Those of skill in the art are familiar with searching databases like NCBI and Swiss Protein. In a preferred embodiment, values are set with a minimum matching peptide setting of 4, mass tolerance settings of 50-250 ppm, and for a single trypsin miss-cut.

Binding Partner Identification

One method for identifying proteins that bind to Akt1 includes: providing a library and selecting from the library one or more members that encode a protein that binds to the Akt1 antigen. The selection can be performed in a number of ways. For example, the library can be a display library. Akt1 can be tagged and recombinantly expressed. The Akt1 is purified and attached to a support, e.g., to affinity beads, or paramagnetic beads or other magnetically responsive particles. Akt1 can also be expressed on the surface of a cell. Members of the display library that specifically bind to the cell can be selected.

In one embodiment, a display library is used to identify proteins that bind to Akt1. A display library is a collection of entities; each entity includes an accessible protein component and a recoverable component (e.g., a nucleic acid) that encodes or identifies the protein component. The protein component can be of any length, e.g. from three amino acids to over 300 amino acids. In a selection, the protein component of each member of the library is probed with Akt1 protein and if the protein component binds to Akt1, the display library member is identified, e.g., by retention on a support. The display libraries can be constructed from cDNAs derived from tissue derived from the transgenic animals of the present invention wherein the accessible protein components are encoded by the cDNAs. The cDNAs contained in the display library may be the result of cDNA subtractions or other cDNA identification procedures outlined above. Thus, the cDNAs may be the result of comparing induced relative to non-induced transgenics or the result of comparing induced transgenics of varying genetic backgrounds or any other comparison of gene expression relating to the methods of the present invention.

Retained display library members are recovered from the support and analyzed. The analysis can include amplification and a subsequent selection under similar or dissimilar conditions. For example, positive and negative selections can be alternated. The analysis also can include determining the amino acid sequence of the protein component and purification of the protein component for detailed characterization.

A variety of formats can be used for display libraries. Examples include the following.

Phage Display. One format utilizes viruses, particularly bacteriophages. This format is termed “phage display.” The protein component is typically covalently linked to a bacteriophage coat protein. The linkage results form translation of a nucleic acid encoding the protein component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage fl, fd, and M13) as well as other bacteriophage (e.g., T7 bacteriophage and lambdoid phages; see, e.g., Santini (1998) J. Mol. Biol. 282:125-135; Rosenberg et al. (1996) Innovations 6:1-6; Houshmet al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof also can be used (see, e.g., WO 00/71694). In one embodiment, the fusion is to a domain of the gene III protein, e.g., the anchor domain or “stump,” (see, e.g., U.S. Pat. No. 5,658,727 for a description of the gene III protein anchor domain). It also is possible to physically associate the protein being displayed to the coat using a non-peptide linkage, e.g., a non-covalent bond or a non-peptide covalent bond. For example, a disulfide bond and/or c-fos and c-jun coiled-coils can be used for physical associations (see, e.g., Crameri et al. (1993) Gene 137:69 and WO 01/05950).

Bacteriophage displaying the protein component can be grown and harvested using standard phage preparatory methods, e.g. PEG precipitation from growth media. After selection of individual display phages, the nucleic acid encoding the selected protein components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, and spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and WO 03/029456, which describes a yeast display system that can be used to display immunoglobulin proteins such as Fab fragments and the use of mating to generate combinations of heavy and light chains.

In one embodiment, diverse nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30; and Schaffitzel et al. (1999) J Immunol Methods. 231(1-2):119-35.

Polypeptide-Nucleic Acid Fusions. Another format utilizes polypeptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the protein component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

ELISA. Proteins encoded by a display library can also be screened for a binding property using an ELISA assay. For example, each protein is contacted to a microtitre plate whose bottom surface has been coated with the target, e.g., a limiting amount of the target. The plate is washed with buffer to remove non-specifically bound polypeptides. Then the amount of the protein bound to the plate is determined by probing the plate with an antibody that can recognize the polypeptide, e.g., a tag or constant portion of the polypeptide. The antibody is linked to an enzyme such as alkaline phosphatase, which produces a colorimetric product when appropriate substrates are provided. The protein can be purified from cells or assayed in a display library format, e.g., as a fusion to a filamentous bacteriophage coat. Alternatively, cells (e.g., live or fixed) that express the target molecule, e.g., Akt1, e.g., constitutively active isoform of Akt1, can be plated in a microtitre plate and used to test the affinity of the peptides/antibodies present in the display library or obtained by selection from the display library.

In another version of the ELISA assay, each polypeptide of a diversity strand library is used to coat a different well of a microtitre plate. The ELISA then proceeds using a constant target molecule to query each well.

Homogeneous Binding Assays. The binding interaction of candidate protein with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate protein is attached to the display library vehicle, e.g., a bacteriophage or using a candidate protein as free molecule.

Surface Plasmon Resonance (SPR). The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (Kd), and kinetic parameters, including Kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

Protein Arrays. Polypeptides identified from the display library can be immobilized on a solid support, for example, on a bead or an array. For a protein array, each of the polypeptides is immobilized at a unique address on a support. Typically, the address is a two-dimensional address. See, for example, MacBeath et al., 2000, Science, 289:1760-1763 and Bertone et al. 2005, FEBS Journal, 272:5400-5411.

Cellular Assays. Candidate polypeptides can be selected from a library by transforming the library into a host cell; the library could have been previously identified from a display library. For example, the library can include vector nucleic acid sequences that include segments that encode the polypeptides and that direct expression, e.g., such that the polypeptides are produced within the cell, secreted from the cell, or attached to the cell surface. The cells can be screened or selected for polypeptides that bind to the Akt1, e.g., as detected by a change in a cellular phenotype or a cell-mediated activity. For example, in the case of an antibody that binds to Akt1, the activity may be an in vitro assay for cell invasion. In one embodiment, the antibody is contacted to an invasive mammalian cell, e.g., a carcinoma cell, e.g., JEG-3 (choriocarcinoma) cell. The ability of the cell to invade a matrix is evaluated. The matrix can be an artificial matrix, e.g., Matrigel, gelatin, etc., or a natural matrix, e.g., extracellular matrix of a tissue sample, or a combination thereof. For example, the matrix can be produced in vitro by a layer of cells.

### Differential Expressed Genes and Gene Products Identified

Determination of the human homologs of the gene transcripts and proteins identified by the methods of the present invention may be easily ascertained by the skilled artisan. “Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application.

In one embodiment, the term “human homolog” to a gene transcript identified as associated with muscle growth refers to a DNA sequence that has at least about 55% homology to the full length nucleotide sequence of the sequence of the gene transcript identified as associated with muscle growth as encoded by the genome of the transgenic animal of the present invention. In one embodiment, the term “human homolog” to a protein identified as associated with muscle growth refers to an amino acid sequence that has 40% homology to the full length amino acid sequence of the protein identified as associated with muscle as encoded by the genome of the transgenic animal of the present invention, more preferably at least about 50%, still more preferably, at least about 60% homology, still more preferably, at least about 70% homology, even more preferably, at least about 75% homology, yet more preferably, at least about 80% homology, even more preferably at least about 85% homology, still more preferably, at least about 90% homology, and more preferably, at least about 95% homology. As discussed above, the homology is at least about 50% to 100% and all intervals in between (i.e., 55%, 60%, 70%, 75%, 80%, 85%, 90%, 95%, 98%, etc.).

Characterization of Differentially Expressed Gene and/or Gene Products.

In some embodiments, the differentially expressed genes and/or proteins are characterized based on their functionality (functional characterization) and structure (structural characterization). In some embodiments, structural characterization is based, in part, on the identification of specific structural motifs, for example the presence of a signal peptide and/or absence of transmembrane domain indicates the gene and/or gene product is likely to act as a muscle secreted protein (herein referred to as “MSP”. In alternative embodiments, in particular differentially expressed gene identified on analysis of the transgenic animals of the present invention, the presence of a transmembrane domain and/or cytoplasmic domain indicates the gene and/or gene product is likely to act as a receptor for a secreted muscle related ligand and/or MSP.

In some embodiments, functional characterization is based, in part, on the likely function of the identified gene and/or gene product. In some embodiments, functional analysis can be determined in silco, for example but not limited to associating a particular function to a gene and/or gene product based on homology and/or association with a particular cellular pathway. In alternative embodiments, functional characterization can be determined by functional assays, for example but not limited to (i) muscle growth and/or muscle regeneration (ii) angiogenesis and (iii) glucose and/or and insulin sensitivity, (iii), obesity and (iv) muscle hypertophy, as disclosed in the Examples and discussed in more detail below. In such embodiments, genes and gene products identified to have desirable functional characteristics are potential therapeutic targets for a variety of disease and disorders associated with muscle-related and/or muscle-associated diseases and disorders, for example, muscle growth, angiogenesis, obesity, insulin sensitivity, insulin-dependent disorders, body weight and/or cardiovascular function

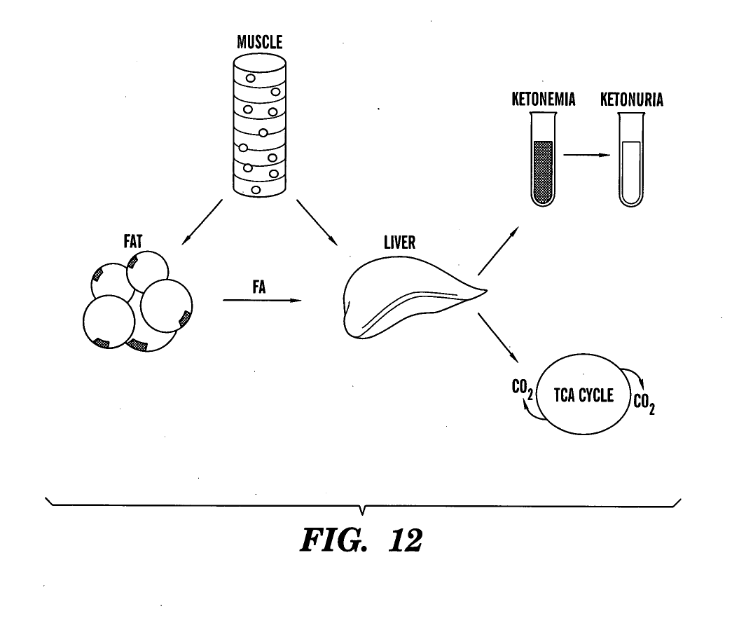

Muscle growth and/or muscle regeneration. Activation of satellite cells in muscle tissue can result in the production of new muscle cells in a subject (For example see FIGS. 12, 30 and 40). Muscle growth, also referred to herein as muscle regeneration refers to the process by which new muscle fibers form from muscle progenitor cells. A gene or gene product that is associated with muscle growth will usually confer art increase in the number of new fibers by at least 1%, more preferably by at least 20%, and most preferably by at least 50%. The growth of muscle may occur by the increase in the fiber size and/or by increasing the number of fibers. The growth of muscle may be measured by an increase in wet weight, an increase in protein content, an increase in the number of muscle fibers, an increase in muscle fiber diameter; etc. An increase in growth of a muscle fiber can be defined as an increase in the diameter where the diameter is defined as the minor axis of ellipsis of the cross section.

In some embodiments, the muscle growth associated with a gene or gene product can be compared with the muscle growth associated with a positive control. In some embodiments, the positive control is an inhibitor of myostatin, for example a neutralizing antibody of myostatin or inhibitory nucleic acid of myostain, for example RNAi of myostatin.

Muscle growth may also be monitored by the mitotic index of muscle. For example, cells may be exposed to a labeling agent for a time equivalent to two doubling times. The mitotic index is the fraction of cells in the culture which have labeled nuclei when grown in the presence of a tracer which only incorporates during S phase (i.e., BrdU) and the doubling time is defined as the average S time required for the number of cells in the culture to increase by a factor of two. Productive muscle regeneration may be also monitored by an increase in muscle strength and agility.

Muscle growth may also be measured by quantitation of myogenesis, i.e. fusion of myoblasts to yield myotubes. An effect on myogenesis results in an increase in the fusion of myoblasts and the enablement of the muscle differentiation program. For example, the myogenesis may be measured by the fraction of nuclei present in multinucleated cells in relative to the total number of nuclei present. Myogenesis may also be determined by assaying the number of nuclei per area in myotubes or by measurement of the levels of muscle specific protein by Western analysis.

The survival of muscle fibers may refer to the prevention of loss of muscle fibers as evidenced by necrosis or apoptosis or the prevention of other mechanisms of muscle fiber loss. Muscles can be lost from injury, atrophy, and the like, where atrophy of muscle refers to a significant loss in muscle fiber girth.

In some embodiments, a gene and/or gene that is associated with muscle growth will usually confer an increase in the number of new fibers by at least 1%, more preferably by at least 20%, and most preferably by at least 50%. In such embodiments, such a gene and/or gene target is exemplified in Example 7, for example Insl6 functions to promote muscle growth by increasing the number of satellite cells and thus increasing the number of new muscle fibers.

Characterization of muscle growth and/or muscle regeneration is exemplified in Example 7. In some embodiments, increase in satellite cells is determined by analysis of muscle tissue by histological methods, and assessing increase in satellite cells, as well as in increase in BrdU incorporation in satellite cells surrounding myofibrils. In alternative embodiments, immunostaining for activated satellite markers can be done, for example immunostaining for MyoD, as demonstrated in Example 7. A gene and/or gene product associated with muscle growth will is identified by its ability to increase the number of satellite cells compared to a muscle in the absence of a gene that promotes muscle growth. Additionally, a gene and/or gene product associated with muscle growth is identified to improve muscle regeneration after a muscle degeneration injury, for example after intramuscular administration of cardiotoxin (CTX) compared to a muscle in the absence of a gene that promotes muscle growth, for example see FIGS. 38 and 38.

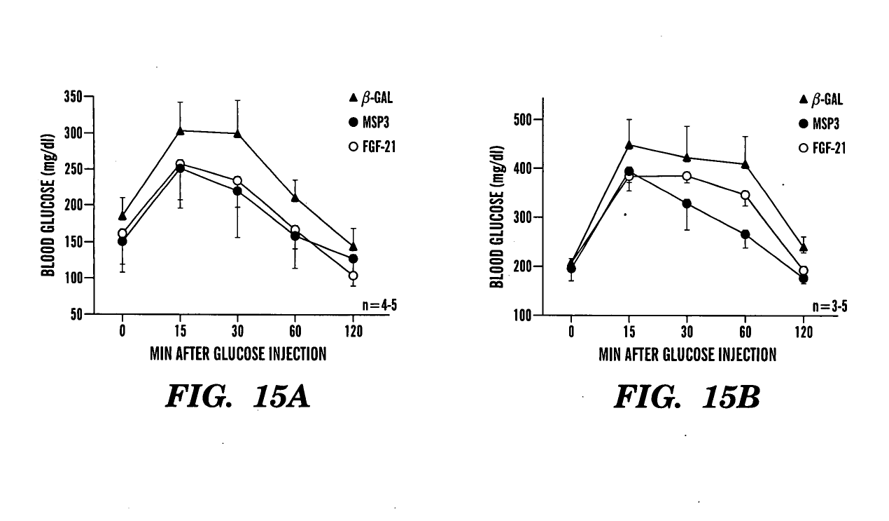

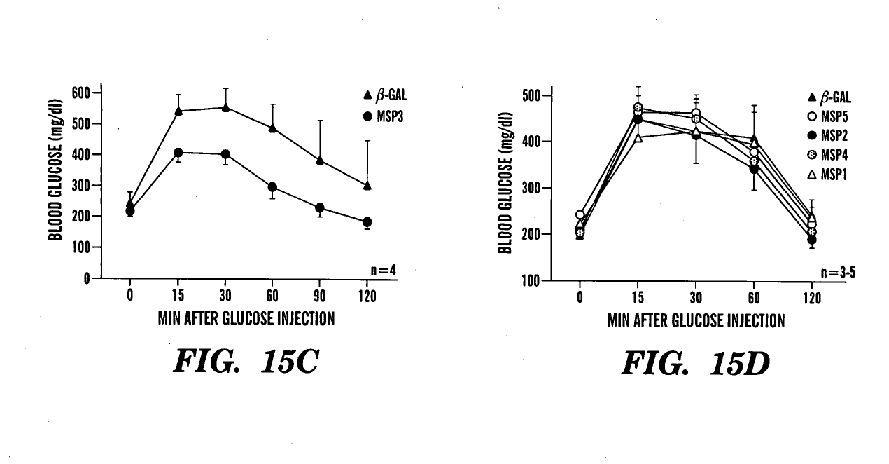

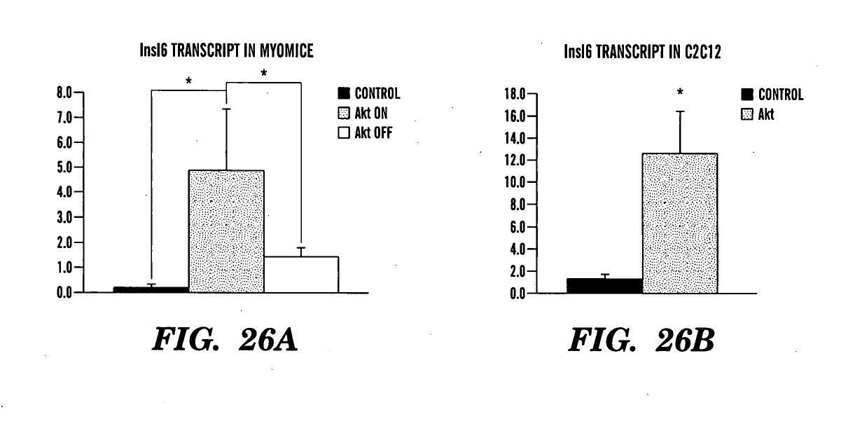

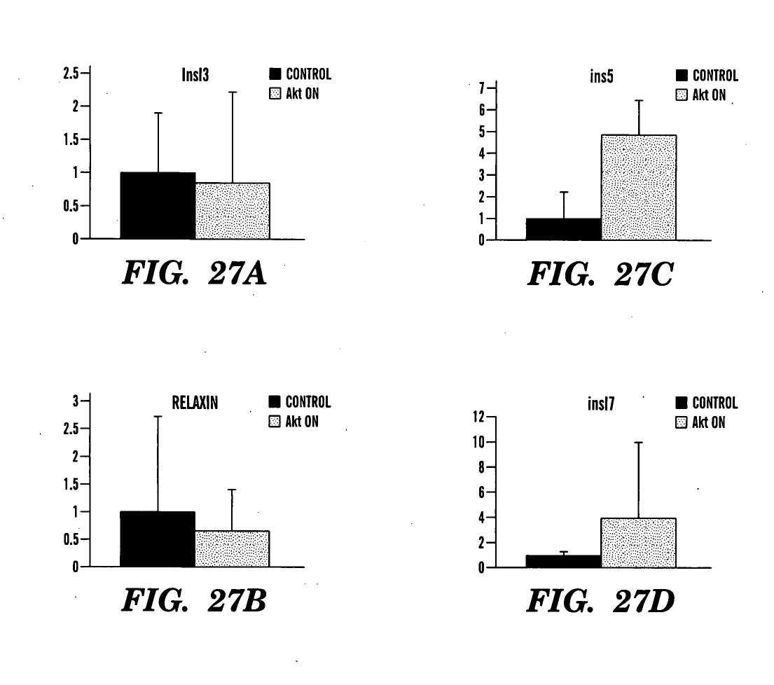

Angiogenesis. The present invention also provides methods to characterize the genes and/or gene products identified by the methods of the present invention with respect to their ability to promote angiogenesis. Methods to assess the ability of a gene and/or gene product to promote angiogenesis are well known to persons of ordinary skill in the art, and include for example, but are not limited to in vitro anlaysis of endothelial cell migration, proliferation, survival, nitric oxide production, or any model of ischemia and/or oxygen deprivation which is routinely used in the art and are commonly known by persons of ordinary skill in the art are encompassed for use in the present invention. An exemplary example of assessing the ability of a gene and/or gene product to promote angiogenesis is its ability to promote revascularization and/or neovascularization in a model of ischemia. Use of such a model is describe in Examples 5 and 6 (see also FIG. 27) where mice are subjected to unilateral hind limb surgery (J. Biol. Chem. 2004; 279:28670-28674; Circ. Res. 2005; 96(8):838-846; Circ. Res. 2006; 98(2):254-61), and the gene of interest is expressed in the muscle of the limb surgery, for example by viral mediated gene expression prior to surgery. Blood vessel growth can be monitored by any means, for example by Laser Doppler analysis on legs and feet immediately before surgery and on postoperative days 0, 3, 7, 14, and 28. A gene and/or gene product associated with promoting angiogenesis or to function as an angiogenic factor is identified to improve blood vessel growth after ischemic limb injury as compared to a muscle in the absence of a gene that promotes angiogenesis for example see FIGS. 15 and 26.

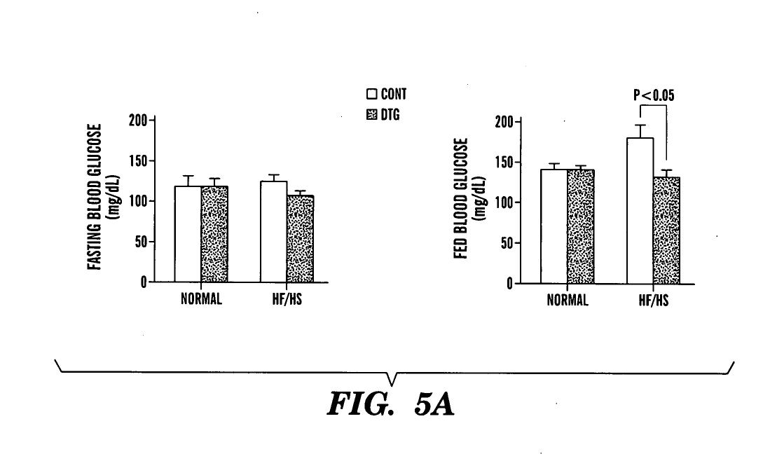

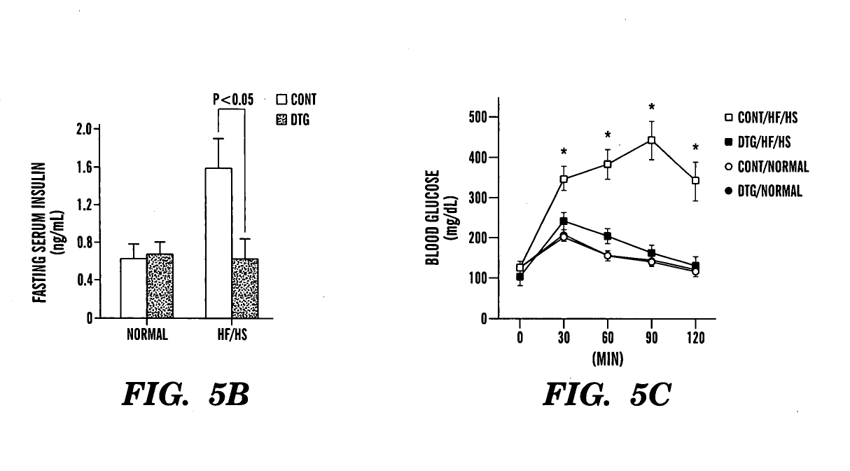

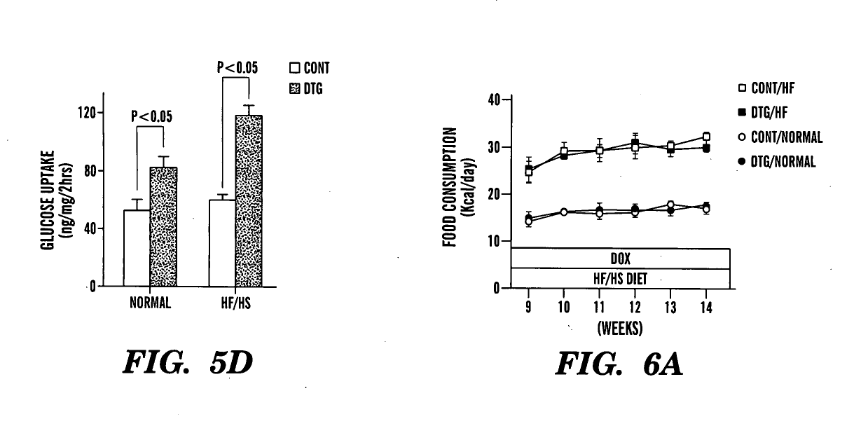

Metabolic regulator. The present invention also provides methods to characterize the genes and/or gene products identified by the methods of the present invention with respect to their ability to function as a metabolic regulator. In particular, a metabolic regulator can be identified on the basis of its ability to regulate glucose and/or insulin sensitivity. An exemplary methods to analyze the ability of a gene and/or gene product to increase sensitivity to glucose and/or insulin is assessment of the blood glucose level after glucose injection in an animal model, for example mice fed a high fat, high sucrose diet (HF/HS) diet to induce obesity in the present or absence of the gene or gene product (for example viral mediated expression of the gene), and blood glucose and blood serum assessed using a glucose tolerance test (GTT). A gene and/or gene product is identified to function as a metabolic regulator if mice fed a HF/HS diet have a lower glucose blood level after injection and/or reduced fasting serum glucose and/or insulin level as compared to a mice in the absence of a gene that functions to increase sensitivity to glucose and/or insulin, for example as discussed in the Examples, such as Example 1 and FIGS. 5, 17 and 18.

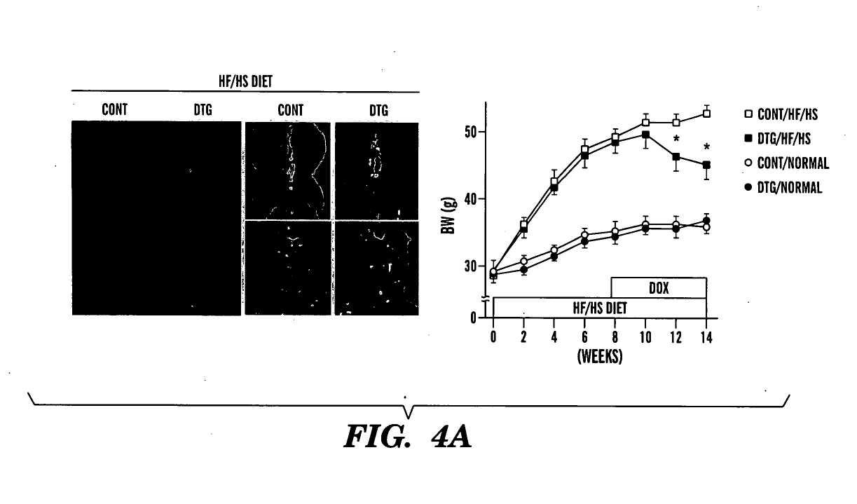

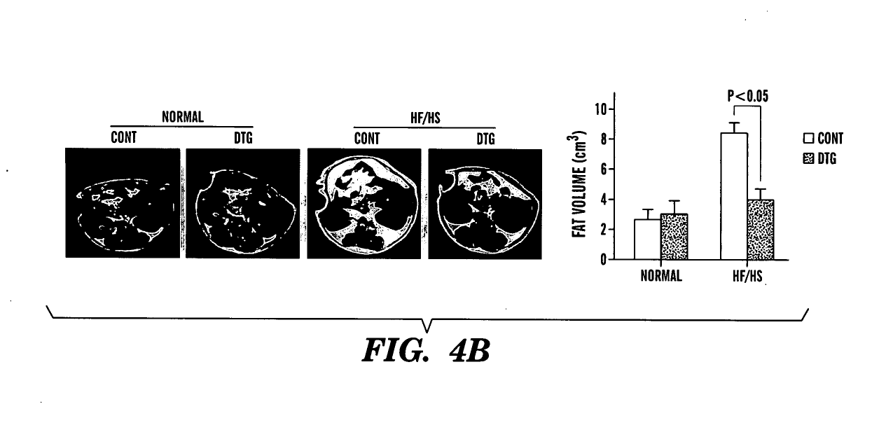

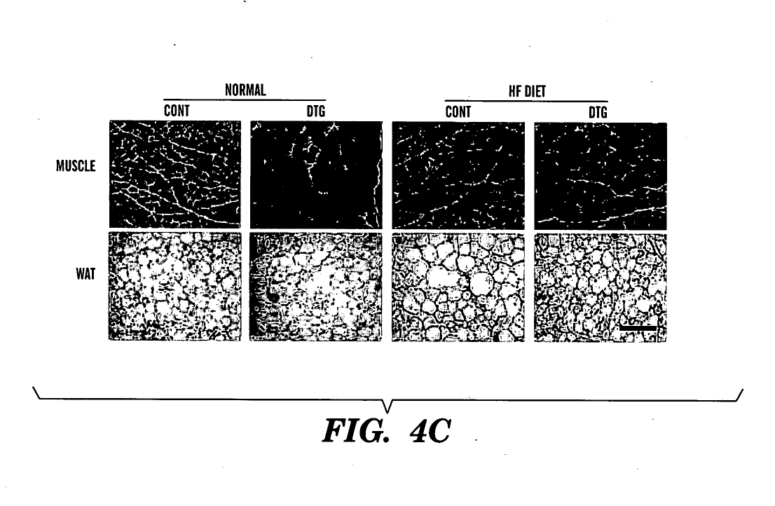

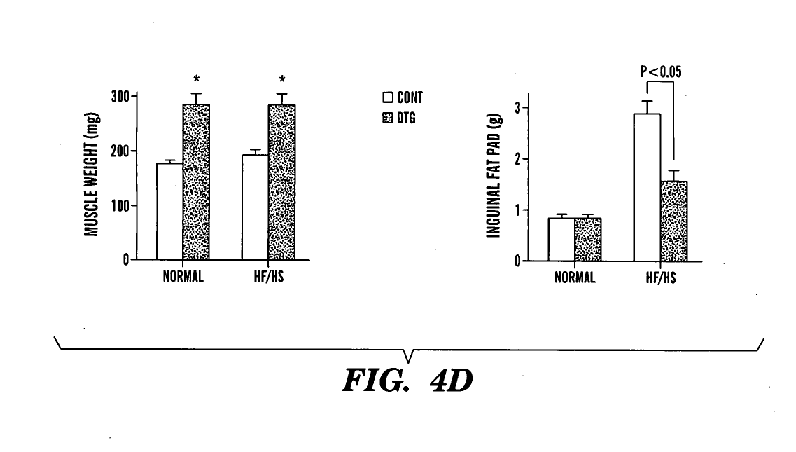

Obesity. The present invention also provides methods to characterize the genes and/or gene products identified by the methods of the present invention with respect to their ability to reduce body weight and fat mass. Exemplary methods to analyze the ability of a gene and/or gene product to reduce body weight and fat mass are described in Example 1, including assessment of total body weight, levels of excess fat by MRI, quantification of adipose cell size, muscle weight, and inguinal fat pad weight in an animal model of obesity, for example mice fed a high fat, high sucrose diet (HF/HS) diet to induce obesity in the presence or absence of the gene or gene product (for example viral mediated expression of the gene). A gene and/or gene product is identified to reduce body mass and/or fat mass if in an obesity animal model, for example mice fed a HF/HS diet, the animals have a lower body weight and/or lower excessive fat as detected by, for example MRI, and/or smaller adipose cell size and/or decreased muscle weight and/or decreased inguinal fat pad weight as compared to a mice in the absence of a gene that functions to increase sensitivity to glucose and/or insulin, for example as discussed in Example 1 and FIG. 4.

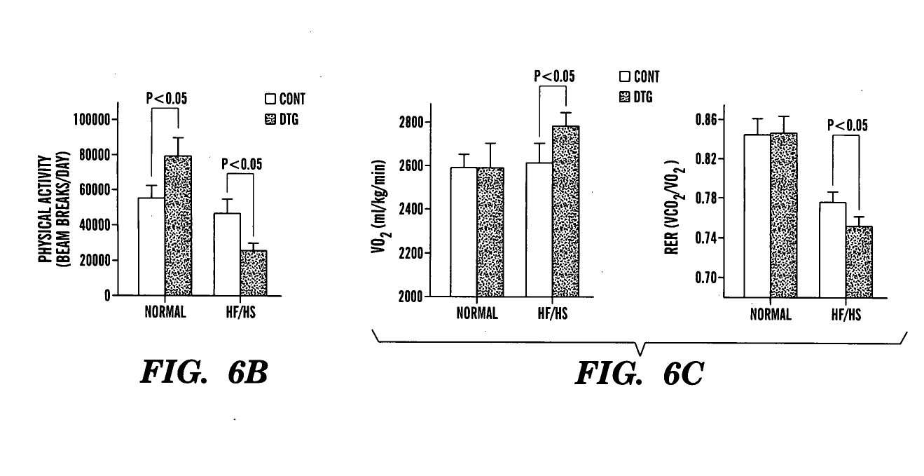

Further characterization to assess the ability of gene and/or gene product to reduce body weight and/or reverse excessive fat accumulation is described in Example 1, (see FIG. 6) can be done by analyzing the energy balance, such as food intake and energy expenditure an animal model of obesity, for example mice fed a high fat, high sucrose diet (HF/HS) diet to induce obesity in the presence or absence of the gene or gene product (for example viral mediated expression of the gene) as discussed in FIG. 6. Measurements of energy intake, such as food and water intake, energy expenditure by whole body O₂ consumption (VO₂) and Respiratory exchange ratio (RER) which reflects the ratio of carbohydrate to fatty acid oxidation can be done, as well as quantitative analysis, for example by quantatiative PCR or QRT-PCR of genes associated with fatty acid oxidation and mitochondrial biogenesis in the skeletal muscle and/or liver. In addition, liver morphology and lipid oxidative function can be analyzed, as well as the effect on HF/HS diet-induced lipid deposition in the liver, as well as serum ketone bodies, which synthesized in the liver and can be used as an indirect marker of hepatic fatty acid oxidation, as well as quantitative analysis of molecules that stimulate fatty acid oxidation in the liver, for example HNF4α, L-CPT1 and PGC1-α. A gene and/or gene product is identified as being capable of reducing body mass and/or fat mass if, in an obesity animal model, for example mice fed a HF/HS diet, the animals have an increased VO2 and/or decreased RER indicating a greater ratio of use of fatty acid as a fuel source, and/or decreased lipid deposition, and/or increased fatty acid oxidation and/or increased serum ketone bodies in the liver and increased expression of markers for fatty acid oxidation in the liver and/or skeletal muscle as compared to a mice in the absence of a gene that functions to increase sensitivity to glucose and/or insulin, for example as discussed in Example 1 and FIG. 4.

Muscle hypertophy and/or myogenic factor. The present invention also provides methods to characterize the genes and/or gene products identified by the methods of the present invention with respect to their ability to increase muscle hypertrophy, for example genes and/or gene products that function as a myogenic factor. Exemplary methods to analyze the ability of a gene and/or gene product to increase muscle cell size is described in Examples 1 and 6. In particular, a myogenic factor and/or muscle hypertrophy factor can be identified on the basis of its ability to increase the size of myofibers in vivo and in vitro, and increases myofiber size and/or width, and increases protein synthesis as disclosed in example 6. A gene and/or gene product is identified to increase muscle hypertrophy, and function as a myogenic factor is the size and/or width of the myofibril increases, and/or the protein synthesis increases in myofibrils in the absence of a gene that functions to increase sensitivity to glucose and/or insulin, for example as discussed in Example 1 and FIGS. 5, 17 and 18.

### Methods of Treatment

The methods of the present invention can be utilized to identify therapeutic agents such as proteins and/or nucleic acids to treat a number of disorders and diseases, for example but not limited to, muscle associated diseases and disorders, muscle growth, angiogenesis, obesity, insulin sensitivity, insuli-dependent disorders, muscle-related diseases and/or cardiovascular function. In one embodiment, a method for treating such disorders comprises administration of an effective amount of a protein identified by the methods of the present invention—Muscular disorders include muscular dystrophy, Duchenne dystrophy; Becker muscular dystrophy; congenital myopathies including nemaline myopathy or myopathy caused by mutations in the gene for the ryanodine receptor; mitochondrial myopathies due to mutations in both mitochondrial and nuclear-encoded genes including progressive external ophthalmoplegia, the Kearns-Sayre syndrome, the MELAS, the MERFF syndrome, infantile myopathy; glycogen storage diseases of muscle including Pompe's disease; channelopathies; myotonic dystrophy (Steinert's disease); myotonia congenita (Thomsen's disease); familial periodic paralysis including hypokalemic (due to mutation in the dihydropyridine receptor-associated calcium channel gene on chromosome 1q) and hyperkalemic form (due to mutation in SCN4A on chromosome 17q).

The inventors have discovered that using the methods of the present invention using either a cell-based and/or transgenic animal model assay expressing a muscle related gene in skeletal muscle, for example Akt1, they are able to identify factors regulated by muscle related proteins that promote (i) muscle growth (i.e. promote muscle hypertopy) for example MSP5 (see Example 6), (ii) angiogenesis, for example MSP3 and MSP5 (see Examples 5 and 6), (iii) increase in glucose and insulin sensitivity, for example MSP3 (see Example 5), and (iv) muscle regeneration and satellite cell recruitment, for example Insl6 (see Example 7).

Accordingly, the present invention relates to methods to identify therapeutic agents to treat disease and/or disorders associated with angiogenesis, insulin insensitivity, muscle degeneration and/or fat mass regulation, insulin-dependent diseases and muscle-related diseases.

In one embodiment, the present invention provides a method for modulating angiogenesis. In one embodiment of the present invention, the invention provides methods to increase angiogenesis by administration of an effective amount of a protein and/or gene or homologue or variant thereof to increase angiogenesis as identified by the methods of the present invention and characterized to improve blood vessel growth, as discussed in the section above entitled “angiogenesis” and also exemplified in Examples 5 and 6. In an alternative embodiment, the present invention also provides a method for reducing angiogenesis by administration of an effective amount of an inhibitor of a gene identified by the methods of the present invention and characterized to improve blood vessel growth.

In another embodiment, the present invention provides a method for modulating muscle mass. In one embodiment, the present invention provides methods to increase muscle mass, comprising administering to a cell an effective amount of a protein and/or gene or homologue or variant thereof to increase muscle growth, muscle mass and/or induce muscle hypertropy as identified by the methods of the present invention and characterized to promote muscle hypertrophy as discussed in the section above entitled “muscle hypertrophy” and also exemplified in Example 6. In an alternative embodiment, the present invention also provides a method for increasing muscle mass by administration of an effective amount of a factor associated with muscle growth identified by the methods of the present invention. In another embodiment, the present invention also provides a method for decreasing muscle mass by administration of an effective amount of an antagonist or inhibitor of a gene identified by the methods of the present invention and characterized to promote muscle hypertrophy.

In yet another embodiment, the present invention provides a method for modulating muscle mass, for example muscle regeneration. In one embodiment of the present invention, the methods provides a method for enhancing muscle regeneration, comprising administering to a cell an effective amount of a protein and/or gene or homologues or functional derivatives thereof that is identified by the methods of the present invention and characterized to increase recruitment of satellite cells to myofibrils as, as discussed in the section above entitled “muscle regeneration” and also in Example 7. The present invention also provides a method for decreasing muscle regeneration by administration of an effective amount of an antagonist or inhibitor to the protein and/or gene that is characterized as increasing the recruitment of satellite cells to myofibrils as identified by the methods of the present invention.

In another embodiment, the present invention provides a method for modulating body weight. In one embodiment of the present invention, methods are provided to promote weight loss, comprising administering to a cell an effective amount of a protein and/or gene or homologue or variant thereof that is identified to increase sensitivity to glucose and/or increase sensitivity of insulin and/or promote weight loss as identified by the methods of the present invention and characterized to increase sensitivity to glucose as discussed in the section above entitled “metabolic regulator” and also in Example 5. The invention also provides a method for promoting weight loss by administration of an effective amount of an agent that functions as an agonist to increase gene expression or activity or the gene and/or gene product identified as a factor associated with increasing sensitivity to glucose and/or insulin as identified by the methods of the present invention.

In yet another embodiment, the present invention provides a method for enhancing insulin sensitivity, comprising administering to a cell an effective amount of a protein that is identified to increase sensitivity to glucose and insulin and/or promote weight loss as identified by the methods of the present invention, as discussed in the section above entitled “metabolic regulator” and also in Example 5. The invention also provides a method for enhancing insulin sensitivity by administration of an effective amount of a factor associated with increasing sensitivity to glucose and/or insulin as identified by the methods of the present invention.

Polypeptides, e.g. a factor associated with muscle growth identified by the methods of the present invention, fragments and derivatives thereof can be obtained by any suitable method. For example, polypeptides can be produced using conventional recombinant nucleic acid technology such as DNA or RNA, preferably DNA. Guidance and information concerning methods and materials for production of polypeptides using recombinant DNA technology can be found in numerous treatises and reference manuals. See, e.g., Sambrook et al, 1989, Molecular Cloning—A Laboratory Manual, 2nd Ed., Cold Spring Harbor Press; Ausubel et al. (eds.), 1994, Current Protocols in Molecular Biology, John Wiley & Sons, Inc.; Innis et al. (eds.), 1990 PCR Protocols, Academic Press.

Alternatively, polypeptides, e.g., a factor associated with muscle growth, or fragments thereof can be obtained directly by chemical synthesis, e.g., using a commercial peptide synthesizer according to vendor's instructions. Methods and materials for chemical synthesis of polypeptides are well known in the art. See, e.g., Merrifield, 1963, “Solid Phase Synthesis,” J. Am. Chem. Soc. 83:2149-2154.

In some embodiments, the polypeptides are modified to increase stability, for example but not limited to, PEGylation or alteration of N- and C-terminal amino acids for altered stability and increased half-life. Such methods are commonly known by persons of ordinary skill in the art and are encompassed for use in the methods of the present invention.

A preformed polypeptide, e.g., a factor associated with muscle growth, can be introduced into a cell using conventional techniques for transporting proteins into intact cells, e.g., by fusing the polypeptide to the internalization peptide sequence derived from Antennapedia (Bonfanti et al., Cancer Res. 57:1442-1446) or to a nuclear localization protein such as HIV tat peptide (U.S. Pat. No. 5,652,122).

Alternatively, the polypeptide, e.g., a factor associated with muscle growth, can be expressed in the cell following introduction of a DNA encoding the protein, e.g., a factor associated with muscle growth, e.g., in a conventional expression vector or by a catheter or by ex vivo transplants.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding the human homologs of factors associated with muscle growth of the present invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the human homolog of the factor associated with muscle growth to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdrl gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In one preferred embodiment, adenovirus vectors are used. In another embodiment, lentiviral vectors are used, such as the HIV based vectors described in U.S. Pat. Nos. 6,143,520; 5,665,557; and 5,981,276, which are herein incorporated by reference.

Use of Adeno-associated virus (AAV) vectors are also contemplated (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals. Such cationic lipid complexes or nanoparticles can also be used to deliver protein. The protein will preferably contain a nuclear localization sequence.

For general reviews of the methods of gene and protein therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

Method for Screening for an Agent that Modulates a Factor Associated with Muscle Growth

The present invention provides for methods to screen for agents that modulate factors associated with muscle growth identified by the methods of the present invention. Transgenic animal models and/or cells expressing a muscle related transgene can also be used to assay test compounds (e.g., a drug candidate) for efficacy on muscle development, muscle growth, obesity, insulin sensitivity and cardiovascular function in test animals, or in samples or specimens (e.g., a biopsy) from the test animals. In some cases, it will be advantageous to measure the markers of muscle growth, obesity, insulin sensitivity and cardiovascular function in samples, blood, which may be obtained from the test animal without sacrifice of the animal.

Test Compounds

The term “compound” or “agent” as used herein and throughout the specification means any organic or inorganic molecule, including modified and unmodified nucleic acids such as antisense nucleic acids, RNAi, such as siRNA or shRNA, peptides, peptidomimetics, receptors, ligands, and antibodies.

In the methods of the present invention, a variety of test agents and physical conditions from various sources can be screened for the ability of the compound to alter expression and/or activity of the factors associated with muscle growth that are identified by the methods of the present invention.

Generally, the effect of an agent s) on a test animal or cell is compared with a test animal or cell in the absence of test compound(s). In cases where the animal is sacrificed, a baseline can be established based on an average or a typical value from a control animals) that have not received the administration of any test compounds or any other substances expected to affect cancer progression and/or metastasis. Once such a baseline is determined, test compounds can be administered to additional test animals, where deviation from the baseline indicates that the test compound had an effect on cancer progression or metastasis.

The test agent can be any molecule, compound, or other substance which can be administered to a test animal. In some cases, the test agent does not substantially interfere with animal viability. Suitable test compounds may be small molecules, biological polymers, such as polypeptides, polysaccharides, polynucleotides, and the like. The test compounds will typically be administered to the animal at a dosage of from 1 ng/kg to 10 mg/kg, usually from 10 μg/kg to 1 mg/kg. Test compounds can be identified that are therapeutically effective, such as anti-proliferative agents, or as lead compounds for drug development.

In some embodiments, test agent can be from diversity libraries, such as random or combinatorial peptide or non-peptide libraries. Many libraries are known in the art, such as, for example, chemically synthesized libraries, recombinant phage display libraries, and in vitro translation-based libraries.

Examples of chemically synthesized libraries are described in Fodor et al. (Science 251:767-73 (1991)), Houghten et al. (Nature 354:84-86 (1991)), Lam et al. (Nature 354:82-84 (1991)), Medynski (Bio/Technology 12:709-10 (1994)), Gallop et al. (J. Med. Chem. 37:1233-51 (1994)), Ohlmeyer et al. (Proc. Natl. Acad. Sci. USA 90:10922-26 (1993)), Erb et al. (Proc. Natl. Acad. Sci. USA 91:11422-26 (1994)), Houghten et al. (Biotechniques 13:412-21 (1992)), Jayawickreme et al. (Proc. Natl. Acad. Sci. USA 91:1614-18 (1994)), Salmon et al. (Proc. Natl. Acad. Sci. USA 90:11708-12 (1993)), International Patent Publication WO 93/20242, and Brenner and Lerner (Proc. Natl. Acad. Sci. USA 89:5381-83 (1992)).

Examples of phage display libraries are described in Scott and Smith (Science 249:386-90 (1990)), Devlin et al. (Science 249:404-06 (1990)), Christian et al. (J. Mol. Biol. 227:711-18 (1992)), Lenstra (J. Immunol. Meth. 152:149-57 (1992)), Kay et al. (Gene 128:59-65 (1993)), and International Patent Publication WO 94/18318.

In vitro translation-based libraries include, but are not limited to, those described in International Patent Publication WO 91/05058, and Mattheakis et al. (Proc. Natl. Acad. Sci. USA 91:9022-26 (1994)). By way of examples of nonpeptide libraries, a benzodiazepine library (see, e.g., Bunin et al., Proc. Natl. Acad. Sci. USA 91:4708-12 (1994)) can be adapted for use. Peptide libraries (see, e.g., Simon et al., Proc. Natl. Acad. Sci. USA 89:9367-71(1992)) can also be used. Another example of a library that can be used, in which the amide functionalities in peptides have been permethylated to generate a chemically transformed combinatorial library, is described by Ostresh et al. (Proc. Natl. Acad. Sci. USA 91:11138-42 (1994)).

The following examples are provided merely as illustrative of various aspects of the invention and shall not be construed to limit the invention in any way.

Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred test compound is a small molecule, nucleic acid and modified nucleic acids, peptide, peptidomimetic, protein, glycoprotein, carbohydrate, lipid, or glycolipid. Preferably, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection, Bethesda, MD, NCI's Developmental Therapeutics Program, or the like.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

The compound formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472.).

Screening compounds for potential effectiveness in modulating transcription and/or protein expression of factors associated with muscle growth can be accomplished by a variety of means well known by a person skilled in the art.

To screen the compounds described above for ability to modulate transcription and/or expression of factors associated with muscle growth, the test compounds should be administered to the test subject. In one embodiment the test subject is a culture of cells comprised of cells derived from muscle, e.g., skeletal muscle. The cells derived from muscle may be a primary cell culture or an immortalized cell line from a normal or a tumorous muscle. In another embodiment, the test subject is an animal with muscle, e.g., skeletal muscle. The animal with muscle can be, but is not limited to, a fruit fly, a frog, a rodent such as a mouse or a rat, a rabbit, a non-human primate, and a human. The muscle derived cells can be obtained from the muscle of a an animal, including but not limited to, fruit fly, a frog, a rodent such as a mouse or a rat, a rabbit, a non-human primate and a human.

The test compounds can be administered, for example, by diluting the compounds into the medium wherein the cell is maintained, mixing the test compounds with the food or liquid of the animal with muscle, topically administering the compound in a pharmaceutically acceptable carrier on the animal with msucle, using three-dimensional substrates soaked with the test compound such as slow release beads and the like and embedding such substrates into the animal, intramuscularly administering the compound, parenterally administering the compound.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The language “pharmaceutically acceptable carrier” is intended to include substances capable of being co-administered with the compound and which allows the active ingredient to perform its intended function of preventing, ameliorating, arresting, or eliminating a disease(s) of the nervous system. Examples of such carriers include solvents, dispersion media, adjuvants, delay agents and the like. The use of such media and agents for pharmaceutically active substances is well known in the art. Any conventional media and agent compatible with the compound may be used within this invention.

The compounds can be formulated according to the selected route of administration. The addition of gelatin, flavoring agents, or coating material can be used for oral applications. For solutions or emulsions in general, carriers may include aqueous or alcoholic/aqueous solutions, emulsions or suspensions, including saline and buffered media. Parenteral vehicles can include sodium chloride, potassium chloride among others. In addition intravenous vehicles can include fluid and nutrient replenishers, electrolyte replenishers among others.

Preservatives and other additives can also be present. For example, antimicrobial, antioxidant, chelating agents, and inert gases can be added (see, generally, Remington's Pharmaceutical Sciences, 16th Edition, Mack, 1980).

Screening for a compound that causes an increase in transcription or protein expression of a factor associated with muscle growth or screening for a compound that ablates activity of a factor associated with muscle growth can be accomplished using measurements of gene transcription and/or measurements of protein expression of the factor associated with muscle growth. Measurements of gene transcription can include direct measurements of gene transcription of the factor associated with muscle growth or measurements of a reporter gene. Similarly, measurements of protein expression can include measurements of protein expression of the factor associated with muscle growth or measurements of a reporter gene.

As noted above, screening assays are generally carried out in vitro, for example, in cultured cells, in a biological sample, e.g., muscle, e.g., skeletal muscle, or fractions thereof. For ease of description, cell cultures, biological samples, and fractions are referred to as “samples” below. The sample is generally derived from an animal (e.g., any of the research animals mentioned above), preferably a mammal, and more preferably from a human.

The reporter gene assay (Tamura, et al., Transcription Factor Research Method, Yodosha, 1993) is a method for assaying the regulation of gene.expression using as the marker the expression of a reporter gene.

Detection and quantification gene expression of the factor associated with muscle growth may be carried out through any of the methods described above in connection with identification of factors associated with muscle growth. Any gene transcription and polypeptide or protein expression assays known to the skilled artisan can be used to detect either the transcription and/or expression of the factor associated with muscle growth. Alternatively, when a reporter gene is utilized, the transcription and/or expression of the reporter gene may also be detected in place of the factor associated with muscle growth utilizing the amplification based, hybridization based and/or polypeptide based assays.

Suitable amplification based methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

Methods of detecting and/or quantifying polynucleotides using nucleic acid hybridization techniques, e.g., Northern Blots, are known to those of skill in the art (see Sambrook et Molecular Cloning: A Laboratory Manual, 2d Ed. vol. 1-3, Cold Spring Harbor Press, NY, 1989). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

Polypeptides of factors associated with muscle growth can be detected and quantified by any of a number of methods well known to those of skill in the art. Examples of analytic biochemical methods suitable for detecting protein include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

Antibodies to the factor associated with muscle growth (preferably anti-mammalian; more preferably anti-human) may be produced by methods well known to those skilled in the art. Fragments of antibodies to the factor associated with muscle growth may be produced by cleavage of the antibodies in accordance with methods well known in the art. For example, immunologically active F(ab′) and F(ab)2 fragments may be generated by treating the antibodies with an enzyme such as pepsin.

### Delivery of Therapeutics

The genes and/or gene products identified by the methods of the present invention, or their homologues, variants, functional derivatives, agonists and antagonists thereof-are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

It should be noted that it can be administered as a compound or as a pharmaceutically acceptable salt and can be administered alone or as an active ingredient in combination with pharmaceutically acceptable carriers, diluents, adjuvants and vehicles.

It is also noted that humans are treated generally longer than the mice or other experimental animals exemplified herein, which treatment has a length proportional to the length of the disease process and drug effectiveness. The doses may be single doses or multiple doses over a period of several days, but single doses are preferred.

When administering the compound of the present invention parenterally, it will generally be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those presented in U.S. Pat. Nos. 5,225,182; 5,169,383; 5,167,616; 4,959,217; 4,925,678; 4,487,603; 4,486,194; 4,447,233; 4,447,224; 4,439,196 and 4,475,196. Other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver the compound orally or intravenously and retain the biological activity are preferred.

In another embodiment, the pharmaceutically acceptable formulations comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used.

Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing an active compound such variables as the efficiency of active compound encapsulation, labiality of the active compound, homogeneity and size of the resulting population of vesicles, active compound-to-lipid ratio, permeability, instability of the preparation, and pharmaceutical acceptability of the formulation should be considered.

Prior to introduction, the formulations can be sterilized, by any of the numerous available techniques of the art, such as with gamma radiation or electron beam sterilization.

When the agents are delivered to a patient, they can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. Administration can be local or systemic as indicated. Agents can also be delivered using viral vectors, which are well known to those skilled in the art.

The pharmaceutically acceptable formulations can be suspended in aqueous vehicles and introduced through conventional hypodermic needles or using infusion pumps.

All publications, including published patent applications and issued patents, mentioned herein are incorporated by reference in their entireties. Having described the invention in general terms, reference is now made to specific examples. It is to be understood that these examples are not meant to limit the present invention, the scope of which is to be determined by the appended claims.

### EXAMPLES

Methods

Skeletal muscle-specific conditional Akt1 TG mice. MCK-rtTA TG mice (Grill et al., 2003) were crossed with Tet-myrAkt1 TG mice (Shiojima et al., 2005) to generate DTG mice. For Akt1 transgene expression, DTG mice were treated with DOX (0.5 mg/ml) in drinking water, and DOX water was removed to repress the transgene expression. MCK-rtTA single TG littermates were used as controls and treated with DOX in the same manner as DTG mice.

Animal care and diet treatments. Study protocols were approved by the Institutional Animal Care and Use Committee at Boston University. Mice were housed at 24° C. on a fixed 12-h light/dark cycle. Mice were fed either a normal chow diet or a high-fat/sucrose diet (HF diet: Diet No. F1850, BIO-SERV) (Harte et al., 1999) as indicated. Food consumption and body weight was monitored daily in individually caged mice.

Physiological measurements. O₂ consumption, CO₂ release rates, and ambulatory activity levels were determined by using a 4-chamber Oxymax system (Columbus Instruments), with 1 mouse per chamber as previously described (Yu et al., 2000). Forced treadmill exercise test was performed by using the treadmill (Columbus Instruments) as previously described (Shalom-Barak et al., 2004). Muscle strength in mice was measured using an automated Grip Strength Meter (Columbus Instruments) as previously described (Acakpo-Satchivi et al., 1997).

MRI measurements. MRI was performed on a Bruker Avance 500 wide bore spectrometer (11.7 T; 500 MHz for proton) fitted with a gradient amplifier for imaging (Viereck et al., 2005). Data were processed with Paravision software provided by the vendor.

Metabolic measurements. Blood glucose was assayed with an Accu-check glucose monitor (Roche Diagnostics Corp.). Serum insulin was determined by enzyme-linked immunosorbent assay, using mouse insulin as a standard (Crystal Chem Inc.). Glucose tolerance tests (GTT) was performed on 6 hours fasted mice. Mice were injected intraperitoneally with D-glucose (1 g/kg of body weight), and blood glucose levels were determined immediately before and at 30, 60, 90, and 120 min after injection. Glucose uptake in vivo skeletal muscle was determined as previously described (Koh et al., 2006). The rate of fatty acid β-oxidation in liver was examined as described previously (Nemoto et al., 2000).

Hindlimb Ischemia model. Mice were anaesthetized with a mixture of ketamine (80 mg/kg) and xyaline (10 mg.kg). The left femoral artery was ligated at the point of entry through the inguinal ligament, at the origin of the popliteal artery, and at midway through the saphenous artery. Small branches were cauterized, and the portion of the artery between the ligatures was removed. Blood flow was measured using a deep penetrating laser Dopper probe (Perimed) placed directly on the gastrocnemius muscle. Flow measurements were made just before, immediately after and at 2, and 4 weeks after femoral arteriectomy. At 4 weeks after femoral resection, gastrocnemius and soleus muscle form the ischemic and control limbs were fixed with methanol and embedded in paraffin. Five micron sections were stained with TRITC-labeled lectin (Bandeiraea Simplicifolia; Sigma-Aldrich).

Histology. Skeletal muscle and liver tissues were embedded in OCT compound (Sakura Finetech USA Inc) and snap-frozen in liquid nitrogen. White adipose tissues were fixed in 10% formalin, dehydrated, embedded in paraffin. Tissue sections were stained with H&E for overall morphology, masson-trichrome (MT) for fibrosis and Oil red-O for lipid deposition by standard methods.

Western blotting. Western blot analysis was performed as described previously (Shiojima et al., 2002). The antibodies used were: phospho-Akt (ser-473) from Cell Signaling Technology; Akt1 and VP16 from Santa Cruz Biotechnology Inc.; HA (12CA5) from Roche Diagnostics Corp.; and tubulin from Calbiochem.

Quantitative real-time PCR. Total RNA from whole gastrocnemius muscle and liver was prepared by Qiagen using protocols provided by the manufacturer. cDNA was produced using ThermoScript RT-PCR Systems (Invitrogen). Real-time PCR was performed as described previously (Izumiya et al., 2006). Transcript levels were determined as the relative number of transcripts to those of 18S rRNA, and normalized to the mean value of control samples. Primer sequences are available upon request.

Statistical Analysis. All data are presented as mean±SEM. Statistical comparison of data from two experimental groups were made by using Student's t test. Comparison of data from multiple groups was made by ANOVA with Fisher's PLSD test. A level of P<0.05 was accepted as statistically significant.

### Example 1

### Skeletal Muscle-Specific Akt1 Transgenic Mice

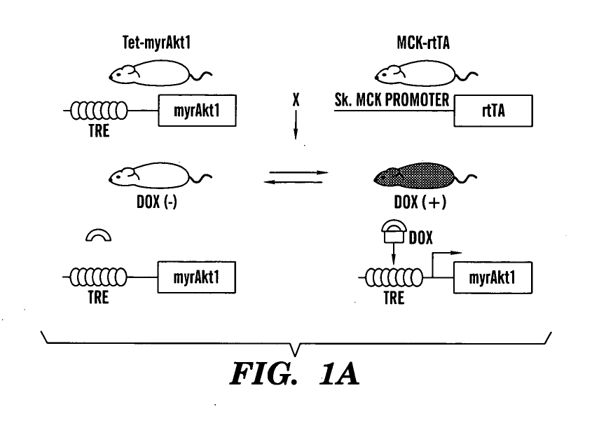

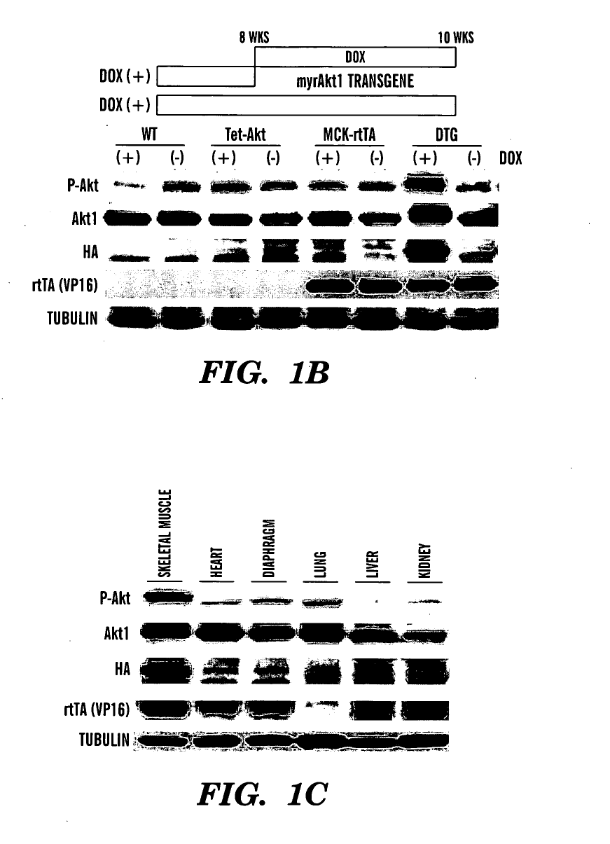

GENERATION OF SKELETAL MUSCLE-SPECIFIC INDUCIBLE AKT1 TG MICE: Two lines of TG mice (Tet-myrAkt1 and MCK-rtTA) were used to generate skeletal muscle-specific conditional Akt1 TG mice (FIG. 1A). Tet-myrAkt1 TG line harbours an active form of Akt1 (myrAkt1) transgene under the control of tetracycline responsive element (TRE) (Shiojima et al., 2005), and MCK-rtTA TG line expresses reverse tetracycline transactivator (rtTA: a fusion protein of TRE and VP16 transactivation domain) in the skeletal muscle driven by mutated MCK promoter (Grill et al., 2003). Treatment of double transgenic (DTG) mice harboring both of two transgenes with doxycycline (DOX) results in myrAkt1 transgene expression because DOX associates with rtTA enable to binding to TRE. On the other hand, withdrawal of DOX inhibited rtTA to bind to TRE and repression of myrAkt1 expression in the skeletal muscle. Mating of Tet-myrAkt1 mice and MCK-rtTA mice resulted in the generation of mice with four different genotypes (wild type (WT), Tet-myrAkt1 TG mice, MCK-rtTA TG mice and DTG mice) in expected frequencies. To examine the regulated expression of Akt1 transgene, these mice were divided into DOX (−) and DOX (+) groups: DOX (+) group was treated with normal water until the age of 8 weeks old followed by DOX treatment for 2 weeks, and DOX (−) group was treated with normal water until the age of 10 weeks old (FIG. 1B). Western blot analysis of gastrocnemius muscle lysates harvested at 10 weeks of age revealed that transgene expression detected by anti-HA blot was observed only in the DTG mice with DOX, indicating that the expression of Akt1 transgene in the skeletal muscle is tightly regulated in a DOX-dependent mariner. The induced expression of Akt1 transgene was associated with marked increase in phosphorylation levels of Akt at Ser473, moderate increase in total Akt protein levels (FIG. 1B). As shown in FIG. 1C, Akt1 transgene expression was detected only in skeletal muscle, indicating that the expression of transgene was regulated skeletal muscle-specific manner. The inventors have also established the second Tet-myrAkt1 transgenic mouse line that exhibits relatively lower expression levels of Akt1 transgene (data not shown). Because the first Tet-myrAkt1 line exhibited more robust growth regulatory effects and permitted a better assessment of skeletal muscle growth and function, further experiments were performed using this line of Tet-myrAkt1 mice.

ACTIVATION OF AKT1 IN SKELETAL MUSCLE CAUSES FUNCTIONAL TYPE II MUSCLE FIBER HYPERTROPHY: To examine the consequence of Akt1 transgene expression in skeletal muscle, mice were treated with DOX at 8 weeks of age and transgene was induced for 2 weeks, and repressed it by removing DOX for 2 weeks. As shown in FIG. 2A, activation of Akt1 signaling for 2 weeks induced robust muscle growth. Akt1-induced muscle growth was completely reversed 2 weeks after withdrawal of DOX. The time course of transgene expression and gastrocnemius muscle weight was examined (FIG. 2B). Akt1 transgene expression was first detected at day 3, which indicated that the initial transgene expression occurred immediately after DOX treatment. Transgene expression reached its maximal level on day 14. Marked repression of transgene expression was observed as early as 2 days after withdrawal of DOX, and transgene expression was completely suppressed by day 14 after withdrawal of DOX. Gastrocnemius muscle weight was significantly increased at day 7, and it was further increased at day 14. When DOX was withdrawal, dramatically repression of hypertrophy was observed at 2 days after withdrawal of DOX. And gastrocnemius muscle weight was almost completely reversed to basal level at 7 days after withdrawal of DOX.

Histological analysis revealed that individual myofiber size was apparently increased, and it was reversed after transgene repression (FIG. 2C). An analysis of the individual fiber sizes demonstrates a significant shift to the right in gastrocnemius muscle. Average cross sectional area was about 2-fold increased 2 weeks after Akt1 activation (2657±195 vs. 1338±180 μm2, p<0.01). Maintaining the transgene expression for 6 weeks induced further skeletal muscle hypertrophy (FIG. 2D). Levels of Akt1 transgene expression was similar extent between 2 weeks and 6 weeks after induction. Gastrocnemius muscle weight was significantly higher than that of control at both time points. As revealed by histology, prolonged Akt1 activation in skeletal muscle induced more pronounced muscle hypertrophy without any pathological change such as interstitial fibrosis or inflammation.

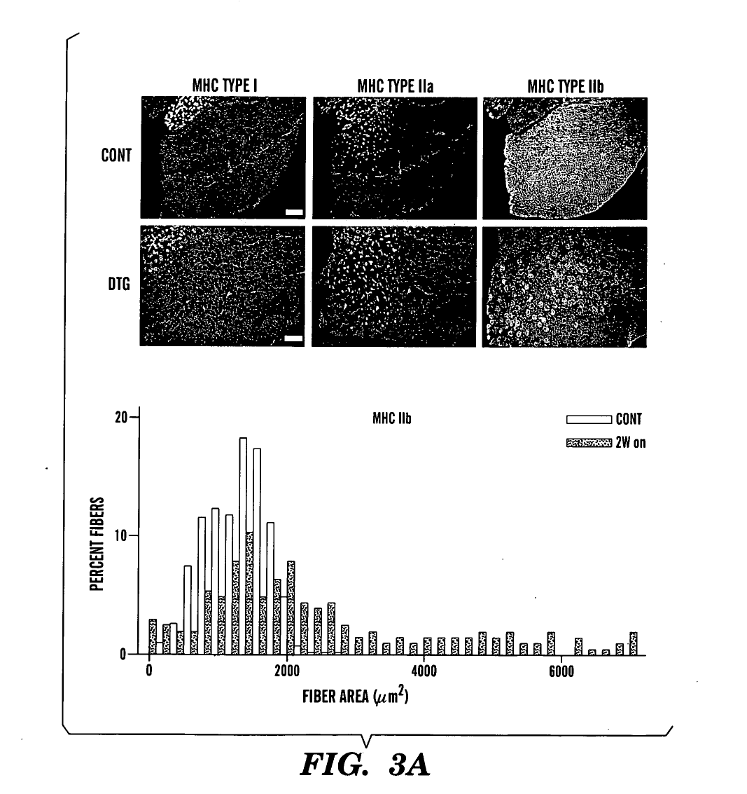

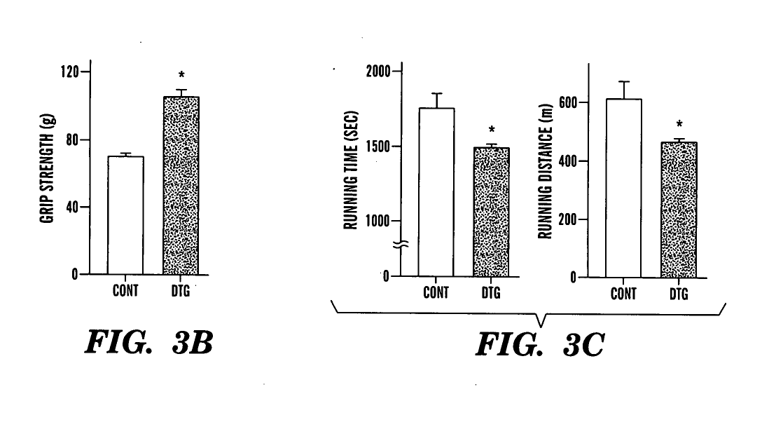

PHYSICAL PERFORMANCE OF AKT-MEDIATED HYPERTROPHIED ADULT SKELETAL MUSCLE: To examine which muscle fiber preferentially express Akt1 transgene, gastrocnemius muscle sections were stained with anti-HA and MHC isoform antibody. As shown in FIG. 3A, Akt1 transgene detected by anti-HA antibody was preferentially expressed in type IIb fibers. Type IIb fibers are classified as fast/glycolytic muscle which is responsible for force generation. Consistent with Akt1 transgene expression profile, the peak grip force for the DTG was about 50% greater than that of control 2 weeks after Akt1 induction (104.7±3.7 vs. 69.8±0.8 g, p<0.05). On the other hand, forced treadmill exercise test revealed that DTG has less running capacity than control (FIG. 3B). These results reveal that Akt1 activation in type II muscle fibers enable to generate of mice strain with “resistance training” phenotype.

AKT1-MEDIATED TYPE II MUSCLE FIBER GROWTH REGRESS DIET-INDUCED OBESITY AND OBESITY-RELATED METABOLIC DISORDERS: To investigate the relationships between type II muscle growth and obesity, mice were fed high fat/sucrose (HF/HS) diet to induce obesity. Under conditions of repressed Akt1 activation, no significant difference was observed in body weight gain in control and DTG mice fed HF/HS diet (FIG. 4A). However, once Akt1 was activated in type II skeletal muscle of obese mice, body weight was dramatically decreased compared with control mice. MRI analysis revealed that accumulation of excessive amount of fat was significantly decreased in DTG mice (FIG. 4B). Histological analysis revealed that myofiber hypertrophy was obvious in DTG mice (FIG. 4C). Adipocyte cell size was enlarged by HF/HS diet in control mice, however, it was apparently smaller in DTG mice. Gastrocnemius muscle weight was significantly increased in DTG mice compared with control mice (FIG. 4D). Inguinal fat pad weight was increase by HF/HS diet in control mice, however, it was dramatically reduced in DTG mice (FIG. 4D).

The inventors next examined the effect of Akt1-mediated type II muscle growth on whole body glucose metabolism. There is no difference in blood glucose levels in each group in fasting period, however, it was significantly higher in HF/HS diet fed control mice in fed period (FIG. 5A). Fasting serum insulin level was significantly increased only in control mice fed HF/HS diet, indicating that these mice developed insulin resistance and Akt1-mediated type H muscle growth improved insulin resistance (FIG. 5B). To investigate this point further, we performed glucose tolerance test (GTT). As shown in FIG. 5C, DTG mice fed a HF/HS diet maintained glucose levels similar to those in mice on normal diet after GTT, whereas control mice fed a HF/HS diet showed higher glucose levels after injection. These results indicate that HF/HS diet-induced severe glucose intolerance was clearly improved after Aka activation in type II skeletal muscle. To examine whether the improved glucose tolerance is owing to increased glucose disposal in skeletal muscle, we measured skeletal muscle glucose uptake in vivo and found that muscle glucose uptake was 1.6-fold and 2.0-fold higher in DTG fed normal diet and HF/HS diet, respectively (FIG. 5D).

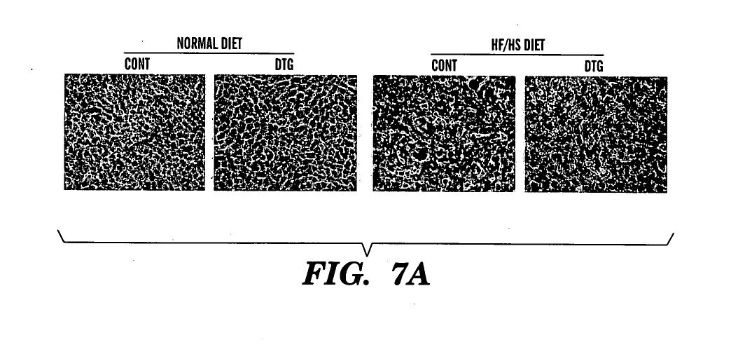

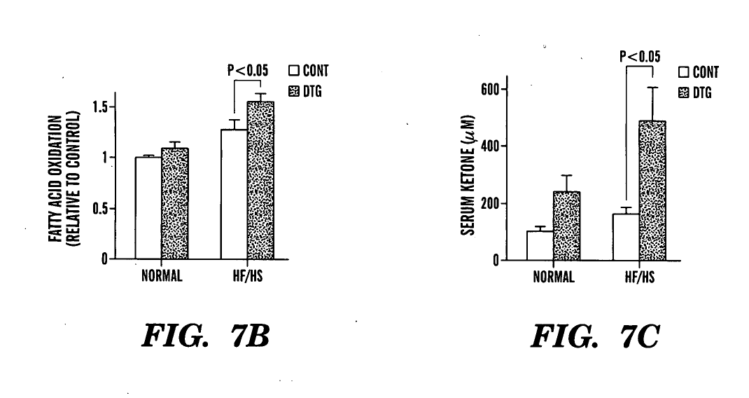

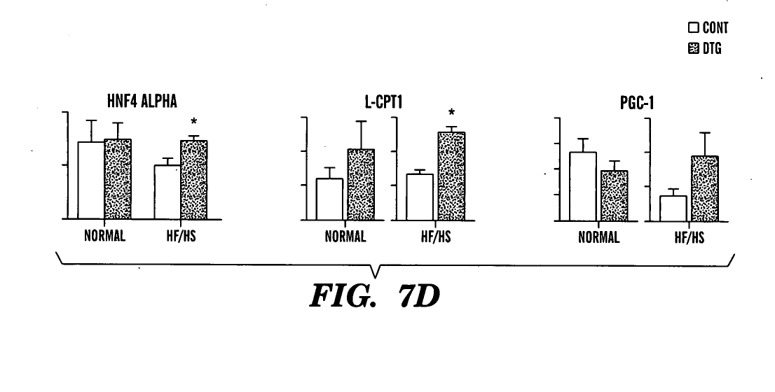

To investigate the mechanism by which excessive fat accumulation was reversed by type II muscle growth, the inventors examined the energy balance: food intake and energy expenditure. Both control and DTG mice show similar food intake throughout the experimental period (FIG. 6A). Although ambulatory activity levels of HF/HS diet fed DTG mice was ˜40% lower than that of control mice (FIG. 6B), energy expenditure estimated by whole-body O2 consumption (VO2) was significantly higher than that of control mice (FIG. 6C). Respiratory exchange ratio (RER), which reflects the ratio of carbohydrate to fatty acid oxidation, was significantly decreased in HF/HS diet fed DTG mice indicating that these mice using a relative greater ratio of fatty acid as a fuel source during the fasting period. However, quantitative real-time PCR analysis revealed that most of genes associated with fatty acid oxidation and mitochondrial biogenesis were not upregulated in skeletal muscle (Table 1). Because liver is a metabolically active organ as well as skeletal muscle, we checked liver morphology and lipid oxidative function. As revealed by histology, HF/HS diet-induced lipid deposition in liver was dramatically resolved in DTG mice (FIG. 7A). To investigate why lipid deposition was decreased in DTG mice, we measured fatty acid oxidation in vivo liver and found that greater fatty acid was oxidized in liver in HF/HS diet fed DTG mice (FIG. 7B). Serum ketone bodies, which synthesized in the liver and can be used as an indirect marker of hepatic fatty acid oxidation, was significantly increased in HF/HS diet fed DTG mice (FIG. 7C). Finally, quantitative real-time PCR analysis showed significant increase in expression of HNF4α, L-CPT1 and PGC1-α in liver in HF/HS diet fed DTG mice, suggesting that Akt1-mediated type II muscle growth activates molecules involved in stimulating fatty acid oxidation in liver.

TABLE 1
Gene Expression associated with energy expenditure in skeletal
muscle in HF/HS fed control or DTG mice.

| Gene | Cont | DTG |
|---|---|---|
| Akt1 | 0.8 ± 0.07 | 9.3 ± 3.92* |
| Cytochrome c | 1.4 ± 0.06 | 1.3 ± 0.04 |
| COX II | 1.4 ± 0.13 | 1.6 ± 0.22 |
| COX IV | 1.8 ± 0.24 | 1.8 ± 0.18 |
| CPT1 | 0.8 ± 0.10 | 1.1 ± 0.09 |
| FATP | 0.9 ± 0.07 | 0.6 ± 0.06 |
| MCAD | 1.0 ± 0.14 | 0.9 ± 0.11 |
| UCP2 | 1.1 ± 0.16 | 4.5 ± 0.82* |
| UCP3 | 0.9 ± 0.14 | 0.9 ± 0.18 |
| PGC1-α | 1.0 ± 0.08 | 0.5 ± 0.05* |
| PPARα | 1.3 ± 0.30 | 0.5 ± 0.03* |
| PPARδ | 1.0 ± 0.04 | 0.8 ± 0.13 |

Values are fold change vs. normal diet control.
*= p < 0.005 vs. HF/HS Diet control.

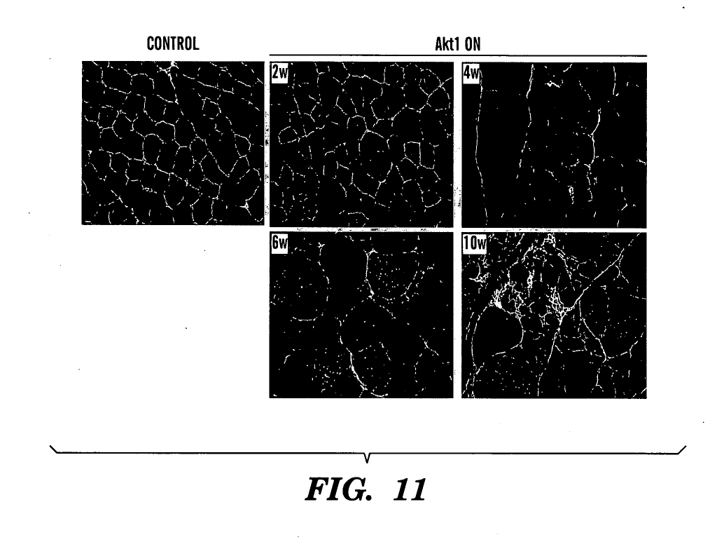

In summary, myogenic Akt transgene activation in obese mice confers the following phenotype: 1) increased muscle mass and strength, 2) diminished fat mass, 3) diminished body weight, 4) improved insulin sensitivity, 5) diminished steatosis (fatty liver), 6) increased angiogenesis in skeletal muscle and 7) increased muscle growth via the incorporation of satellite cells (FIG. 11). These effects occur despite constant levels of food intake and physical activity.

In the present study, the inventors have discovered that type II skeletal muscle growth regress obesity and obesity-related metabolic disorders in obese mice. Akt1 activation in type II skeletal muscle dramatically induced muscle hypertrophy, which was accompanied by an apparent reduction in body weight, especially in fat mass, as well as an improvement of glucose intolerance induced by HF/HS diet. These effects were achieved without dietary and activity modifications. Furthermore, type II skeletal muscle growth led to increased fatty acid oxidation and decreased lipid deposit in liver. Because type II muscle fibers are dramatically decreased upon aging (Larsson, 1983), and cross-sectional studies revealed that muscle strength is inversely correlated with the prevalence of metabolic syndrome (Jurca et al., 2005; Jurca et al., 2004), the inventors have discovered that resistance training aimed at increasing type II muscle fibers is beneficial intervention for the patients, particularly in elderly, with obesity and obesity-related metabolism.

In the present study, the inventors have discovered that type II muscle fiber growth led to increase whole-body energy expenditure independent of physical activity levels in obese mice (FIGS. 6B, C). This discovery indicates that building and maintaining type II muscle per se energy expensive. Reduced energy supplies to adipose tissue as a result of increase energy demand from large skeletal muscles might contribute to regression of obesity. Furthermore, the inventors have discovered that Akt1 activation in type II muscle fibers significantly increased glucose uptake into skeletal muscle, and impaired glucose tolerance induced by HF/HS diet was dramatically improved by Akt1 activation in type II muscle (FIG. 5). Therefore, the.inventors have discovered that Akt1-mediated type II fiber growth leads to improvement of HF/HS diet-induced glucose intolerance. Another remarkable discovery disclosed herein is that HF/HS diet-induced liver seatosis was dramatically resolved and fatty acid oxidation was significantly increased in liver in DTG mice (FIG. 7), indicating that activation of Akt1 signaling in skeletal muscle produces some secreted factor and directly affect liver in a paracrine manner. In a previous study, the inventors have previously reported that Akt1 activation in skeletal or cardiac muscle induced muscle hypertrophy and coordinated blood vessel recruitment by secreting pro-angiogenic factors released from myocyte (Shiojima et al., 2005; Takahashi et al., 2002). The discovery herein indicates that Akt1-mediated type II muscle growth induce coordinated regulation of glucose/lipid metabolism with other organs.

In conclusion, the inventors have discovered for the first time that type II skeletal muscle growth improves obesity-related metabolism by modulating lipid oxidation in liver. This discovery provides a novel concept that type II skeletal muscle fibers regulate glucose/lipid metabolism by communicating remote organs.

### Example 2

### Detailed Characterization of Activation Akt1in Skeletal Muscle

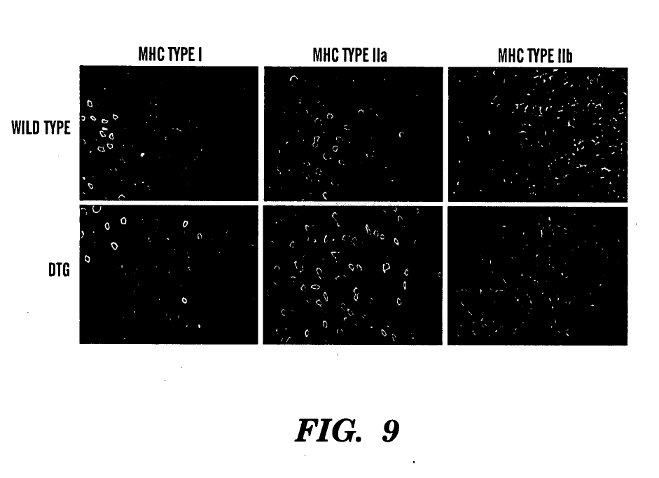

Transgenic mice with inducible expression of Akt in muscle were further characterized, as shown in FIGS. 9-12. When expression of Akt was induced by administration of DOX(DTG) to the drinking water hypertrophy of Type IIb muscle fibers, typically glyolytic/fast twitch fibers is seen compared to wild type mice, and less Type I and Type IIa fibers occur in DOX treated Akt mice compared to control.

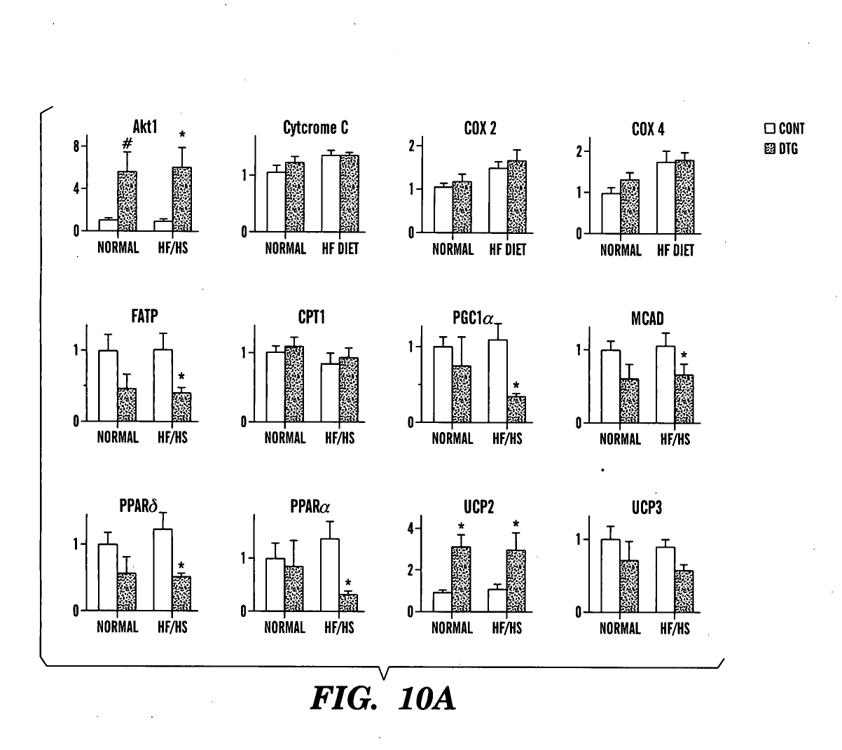

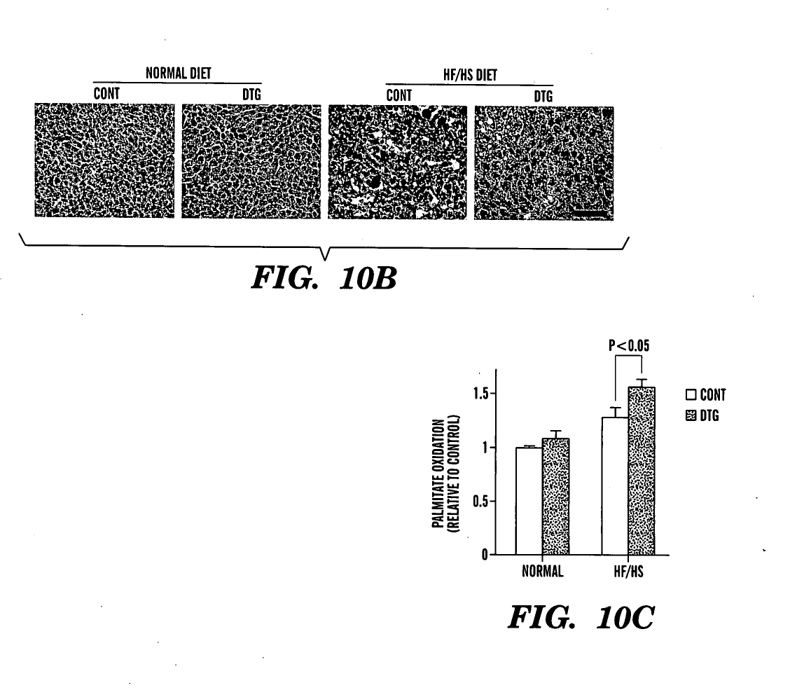

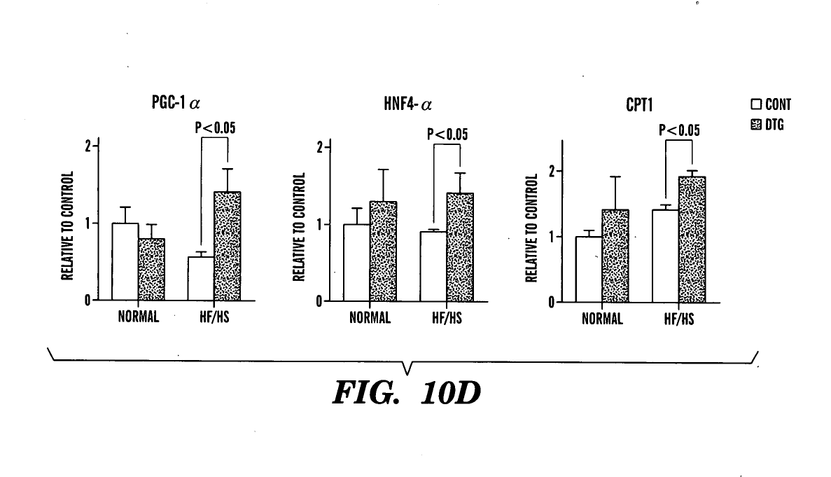

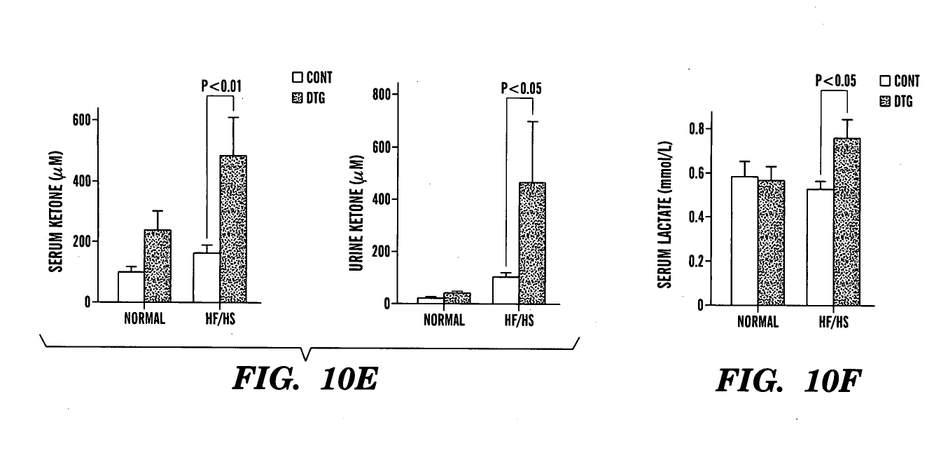

Transgenic Akt mice fed DOX fed high fat and high sugar (HF/HS) also have increased lipid peroxidation in the liver but not muscle compared to control mice fed HF/HS diet, as detected by quantitative gene expression analysis if a number of mRNAs associated with fatty acid oxidation and mitochondrial biogenesis (FIG. 10A) and increase in total fatty acid β-oxidation of palmitic acid (FIG. 10C), and also morpholological analysis of liver using oil red-O stain (FIGS. 7A and 10B). In Akt transgenic mice fed HF/HS diet, PGC-1, HNF4-α and CPT-1, genes associated with fatty acid oxidation are increased (FIG. 10D) as well as increases in increases in serum and urine ketone bodies (FIG. 10E) and serum lacatate levels (FIG. 10F) compared to HF/HS fed control mice, indicating Akt activation in the muscle increases fatty acid metabolism in the liver as well.

Analysis of the effect of induction of Akt expression in the muscle on other tissues can be evaluated using differential gene expression analysis, for example using tussues from liver, adipose cells, and other organs, for example kidney, spleen, pancreas, nervous system tissue etc (see FIG. 12),

### Example 3

INDUCEBLE EXPRESSION OF AKT1 IN VITRO: Transduction of cells in vitro or tissues in vivo with Akt1, Akt2 or Akt3 (constitutively-active or dominant-negative forms) should lead to similar changes in transcript levels because we have shown previously that Akt2 (Fujio Y. et al. 2001 Cell Death Duff 8:1207-1212), and Akt3 (Y. Taniyama 2005 J Mol Cell Cardiol 38:375-385) share function properties with Akt1.

To examine if activation of Akt in skeletal cells in vitro leads to similar changes in gene expression, a myogenic cell line, C2C12 cells, was transduced with an adenovirus expressing Akt1 (Adv-myrAkt). Comparison of gene expression of Adv-myrAkt cells transfected cells 1 day after with skeletal muscle cells of induced expression of Akt1 in skeletal muscle in mice have a highly similar gene expression profile, indicating skeletal muscle cells expressing Akt1 cultured in vitro are effective tools for studying muscle secreted proteins (MSP) or myokines

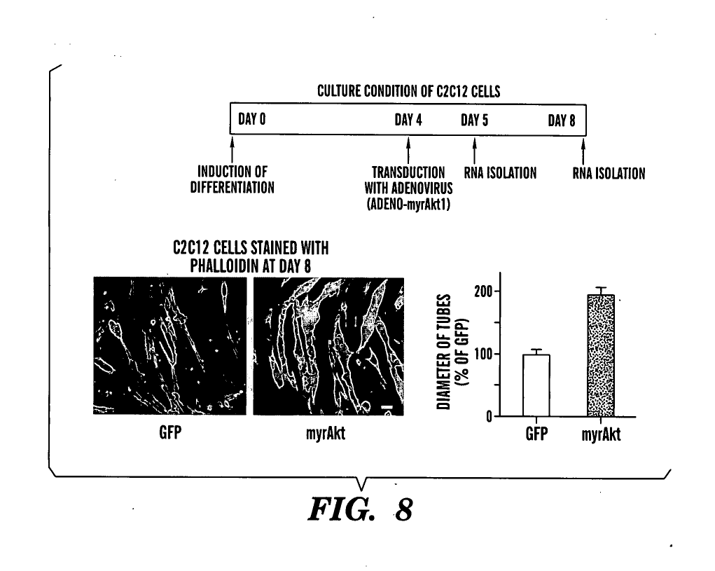

As shown in FIG. 8, the a protocol was devised to identify muscle secreted proteins (MSPs) in an in vitro assay, by expressing myrAkt in a skeletal muscle cell line, for example C2C12. Other skeletal muscle cell lines can be utilized, for example human skeletal muscle cell lines.

### Example 4

IDENTIFICATION OF FACTORS ASSOCIATED WITH MUSCLE GROWTH, ANGLIOGENEIS, OBESITY, INSULIN SENSITIVITY AND CARDIOVASCULAR FUNCTION: A protocol was devised to identify novel muscle secreted proteins (MSPs) that confer the phenotypes, for example, but not limited to increased glucose sensitivity and insulin sensitivity, decreased fat mass and decrease fat cell size, decreased liver deposition, increased capillary density and increased satellite cell recruitment and incorporation of satellite cells into the fibers. First, we performed microarray analysis on muscle of control and DTG mice. Total RNA from gastrocnemius muscle of inducible Akt transgenic mouse (3 groups; no transgene induction (control), 2 weeks induction (2 w on), and 2 weeks induction/2 days repression (2 w on/2 d off)) was analyzed by Affymetrix GeneChip® Mouse Expression Set 430 microarrays. Among the transcripts upregulated by Akt induction, unknown genes were selected which have full-length open reading frame cDNAs available in the NCBI website. Predicted amino acid sequences were then examined for putative signal sequences using Signal IP software. Unknown transcripts with predicted signal sequences were then analyzed with SOSUI signal beta version software to predict whether they encode for secreted proteins versus an integral membrane proteins. This subset of cDNAs was then validated by real-time PCR in the gastrocnemius muscle of DTG mice in the presence or absence of DOX

Based upon the above examination, 8 selected cDNAs were further analyzed to test whether the gene products are secreted by mammalian cells. Full-length cDNAs were obtained by PCR, and subcloned into pcDNA3.1/V5-His that express the unknown protein as a fusion to the V5 epitope in the N-terminus and His tag in C-terminus for detection. These expression vectors were then transfected into HEK293 cells. After 2 days the cell pellets and media fractions were collected and analyze by western blot using anti-V5 antibody where recombinant protein can be detected both in the cell pellet and in the medium, indicating that this cDNA encodes a secreted protein. In all, 6 of the 8 cDNAs encoded secreted proteins as assessed by this HEK293 cell transfection assay, whereas 2 cDNAs did not (i.e. protein could be detected in the cell pellet but not media).

In summary, based upon microarray analysis and transfection assays as described above, the inventors have identified 6 novel cDNAs encoding secreted proteins that are upregulated during Akt-mediated muscle growth (Table 2). These factors are referred to as muscle-secreted proteins 1-6 (MSP 1-6). The Riken identification numbers and the GenBank Accession Numbers for MSP 1-5 are described in Table 2. MSP6 is FGF21, a metabolic factor that may have utility for diabetes and obesity (J. Clin. Invest. 2005; 115:1627-1635).

TABLE 2
Muscle secreted proteins

| | Akt transgene | | | |
|---|---|---|---|---|
| | 2 w | 2 w on/ | | |
| Gene | on | 2 d off | Secretion | Homology |
| MSP 1 | 6.8 | 3.8 | yes | Thrombospondin, type I like |
| MSP 2 | 4.7 | 2.9 | yes | None |
| MSP 3 | 15.1 | 7.5 | yes | Coiled coil domain |
| MSP 4 | 2.4 | 1.1 | yes | peptidase M20 domain |
| MSP 5 | 1.9 | 0.7 | yes | calcium-binding |
| | | | | EGF-like domain |
| MSP 6 | 68.0 | 5.8 | yes | similar to fibulin-1 C (N-term) |
| (FGF21) | | | | |

MSP 1 corresponds to 2610028F08Rik (GenBank Accession No. BC052844) (SEQ ID NO: 16),
MSP 2 corresponds to 2310043I08Rik (GenBank Accession No. AK009779) (SEQ ID NO: 17),
MSP 3 corresponds to 1110017I16Rik (GenBank Accession No. NM_026754) (SEQ ID NO: 1),
MSP 4 corresponds to 4732466D17Rik (GenBank Accession No. BC025830, AK028883) (SEQ ID NO: 18), and
MSP 5 corresponds to 1600015H20Rik (GenBank Accession No. AK005465) (SEQ ID NO: 12).

### Example 5

### MSP3 as Metabolic Regulator

PRODUCTION OF ADENOVIRAL VECTORS EXPRESSING MSPS AND EFFICICY IN ISCHEMIC HIND LIMB ANGIOGENESIS ASSAY: Adenoviral expression vectors to all six MSP cDNAs were produced by homologous recombination in HEK 293 cells as described previously (Mol. Cell. Biol. 2002; 22:680-691). In brief, MSP cDNAs were subcloned into an adenovirus shuttle vector designated Adeno-MSPs. Shuttle vector containing the MSP cDNAs were linearized and cotransformed into Escherichia coli with the adenoviral backbone plasmid pAdEasy-1. The resultant recombinant adenoviral DNA with MSP cDNAs were transfected into packaging cell line 293 cells to produce the recombinant adenoviral vectors. All viral constructs were amplified in 293 cells and purified by CsCl ultracentrifugation that is routine in the lab (Mol. Cell. Biol. 2002; 22:680-691; J. Biol. Chem. 2005; 280:20814-20823).

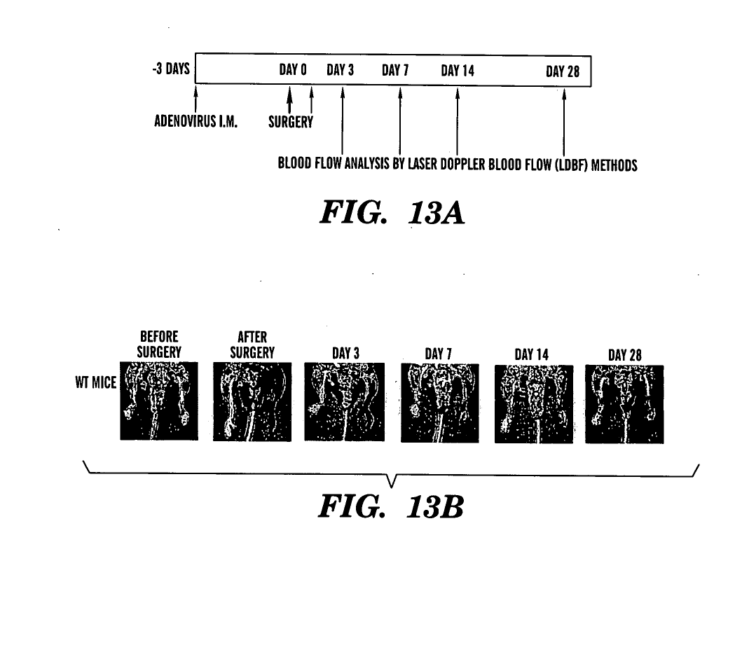

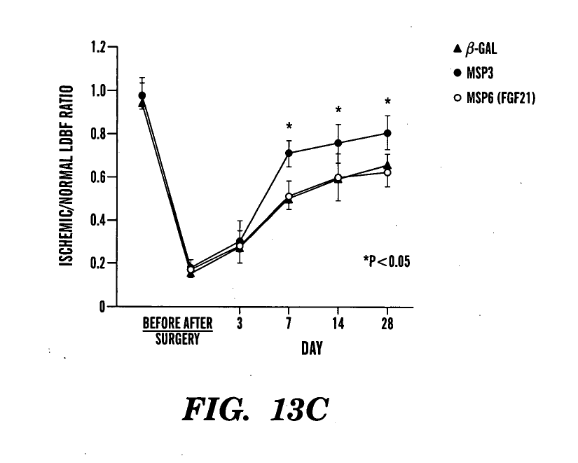

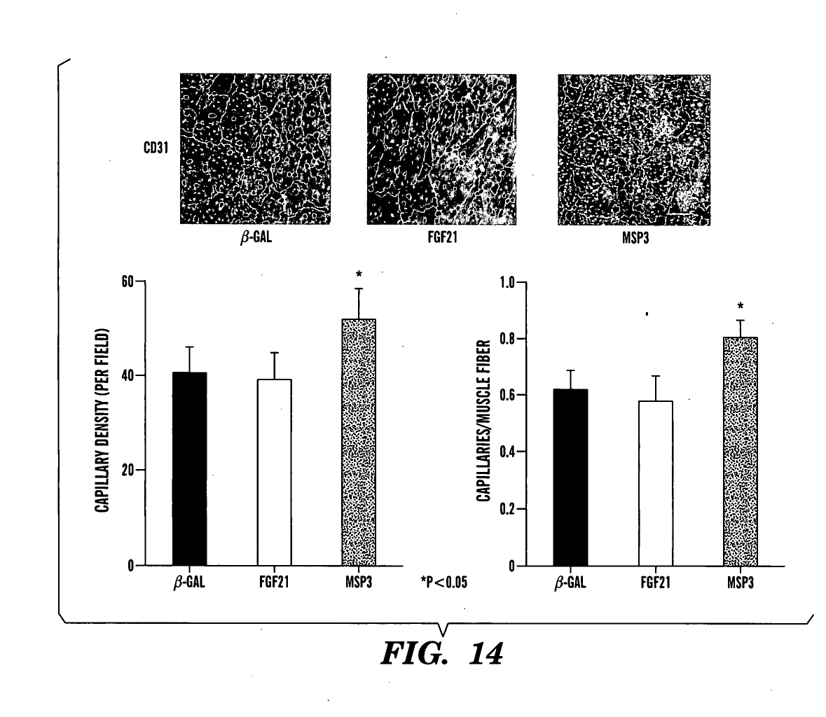

MSP3 corresponds to 1110017I16Rik (GenBank Accession No. NM_{—}026754) (SEQ ID NO:1) and was discovered to be differentially regulated in response to expression of muscle related transgenes and muscle growth. To evaluate the potential angiogenic properties of two MSPs in vivo, MSP3 and MSP6 (FGF-21), mice at the ages of 10 weeks were subjected to unilateral hind limb surgery (J. Biol. Chem. 2004; 279:28670-28674; Circ. Res. 2005; 96(8):838-846; Circ. Res. 2006; 98(2):254-61). Adenovirus-mediated gene transfer was performed with adenoviral vectors expressing MSP3 and MSP6 by direct injection into five different sites of adductor muscle in the ischemic limb 3 days before surgery. Blood vessel growth was monitored by Laser Doppler analysis on legs and feet immediately before surgery and on postoperative days 0, 3, 7, 14, and 28 (FIG. 13). As shown in FIG. 13, adeno-MSP3-treated mice showed a significant increase in flow recovery at 7, 14, and 28 days after hind limb surgery as determined by laser Doppler blood flow analysis. On the other hands, adeno-MSP6 did not affect on flow recovery compared to control mice. These results suggest that MSP3, but not MSP6 (i.e. FGF21), functions as an angiogenesis-regulatory protein. This is also shown in FIG. 14, where Adv-MSP3 improves capillary density and microvessel formation, as identified by CD31 immunostaining, and quantitative analysis of capillary density compared to Adv-FGF21 or control Adv-β-gal treated mice.

MSP3 as a Metabolic Regulator of Glucose Sensitivity and Regresses Diet-Induced Obesity and Obesity-Related Metabolic Disorders:

A protocol to assess diet-induced obesity model to test MSP metabolic function was established, in which mice fed a high fat, high sucrose diet are injected intramuscularily with Adenovirus (Adv) expressing MSP3 (at 1×10¹⁰ pfu) and body weight assessed at 7, 14, 21 and 28 days after Adv-injection, and blood glucose assessed at 14 and 28 days.

On Intramuscular injection of Adeno-MSP3, diet induced obesity mice had improved metabolic response and glucose sensitivity compared to Adv-β-gal injected mice (FIGS. 15A and B). Adenovirus-encoded MSP3 appears functionally equivalent to adenovirus-delivered FGF-21 (also known as MSP6), with blood glucose (mg/dl) returning to the same level with Adv-MSP3 and Adv-FGF21 by 120 minutes after glucose injection. Furthermore, this improved metabolic response and glucose sensitivity was not observed for other MSPS; (MSP5, MSP2, MSP4 and MSP1) (FIG. 15D).

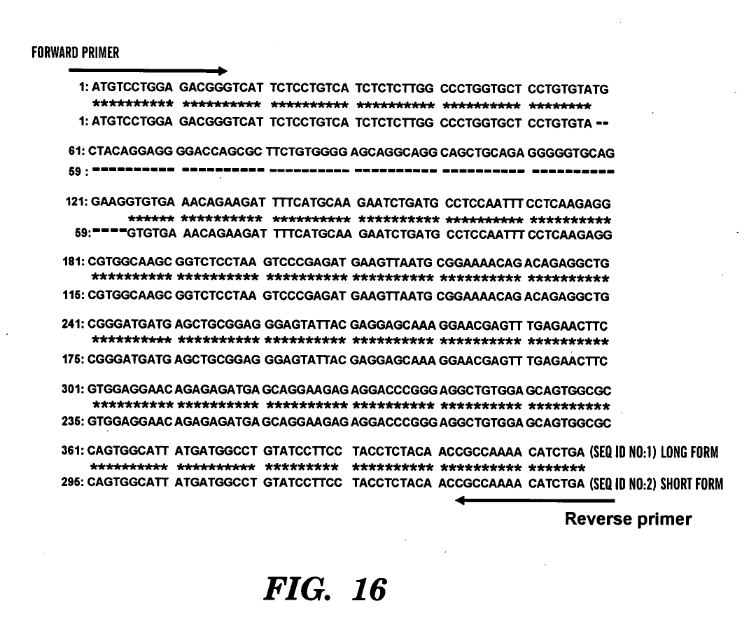

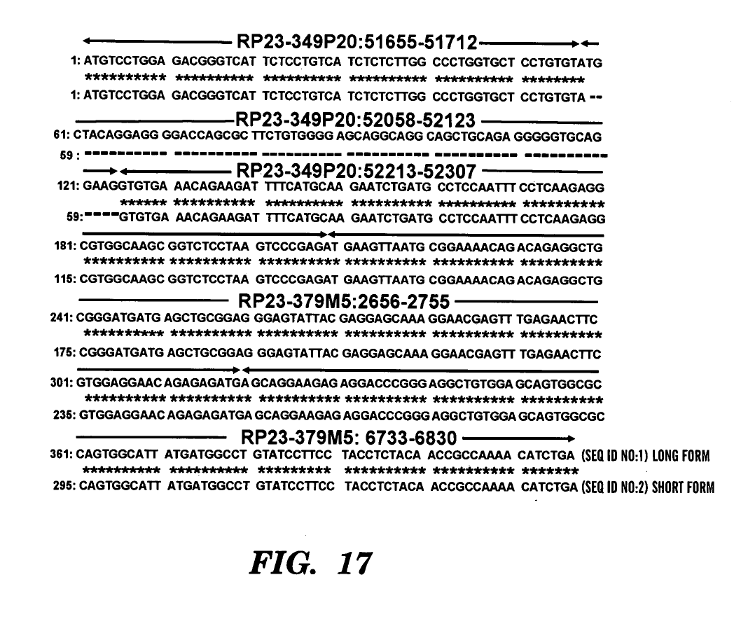

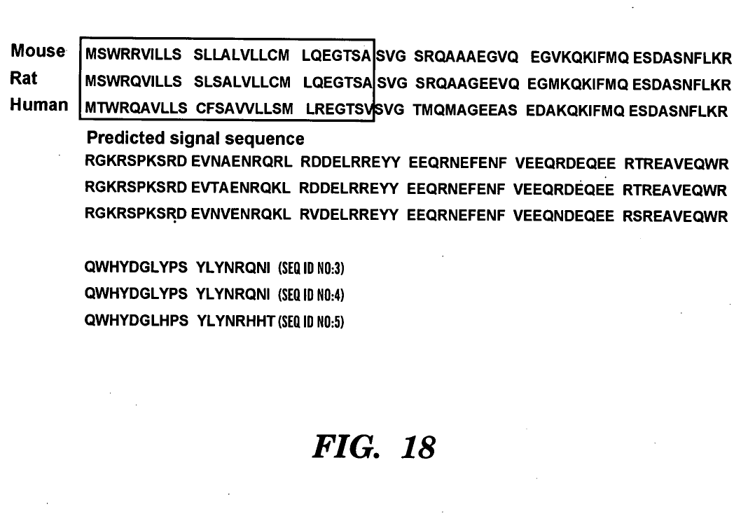

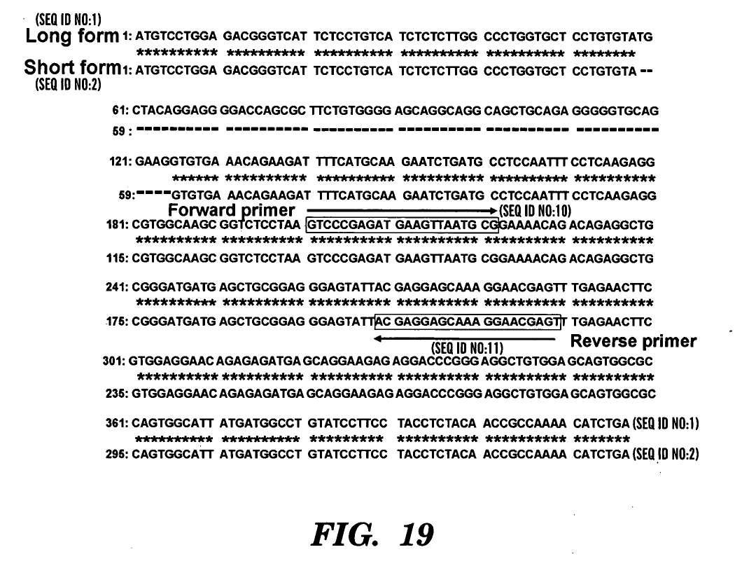

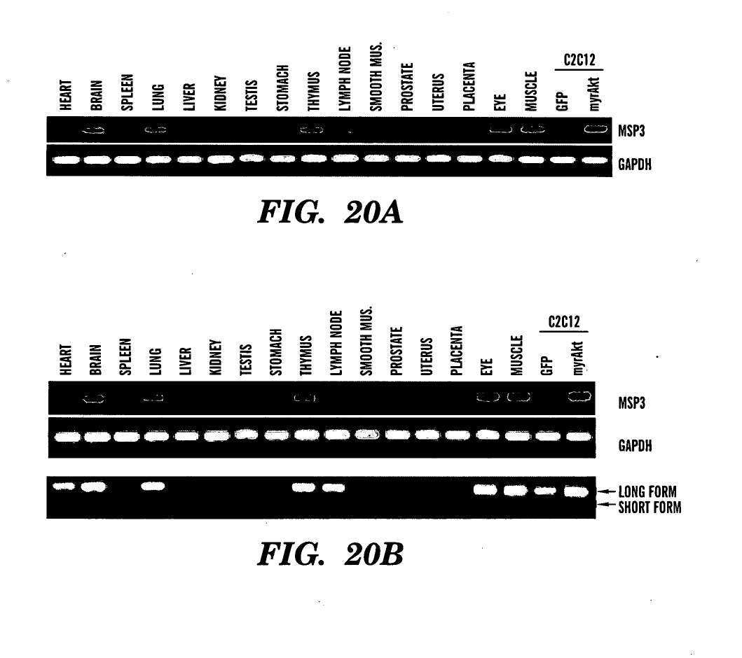

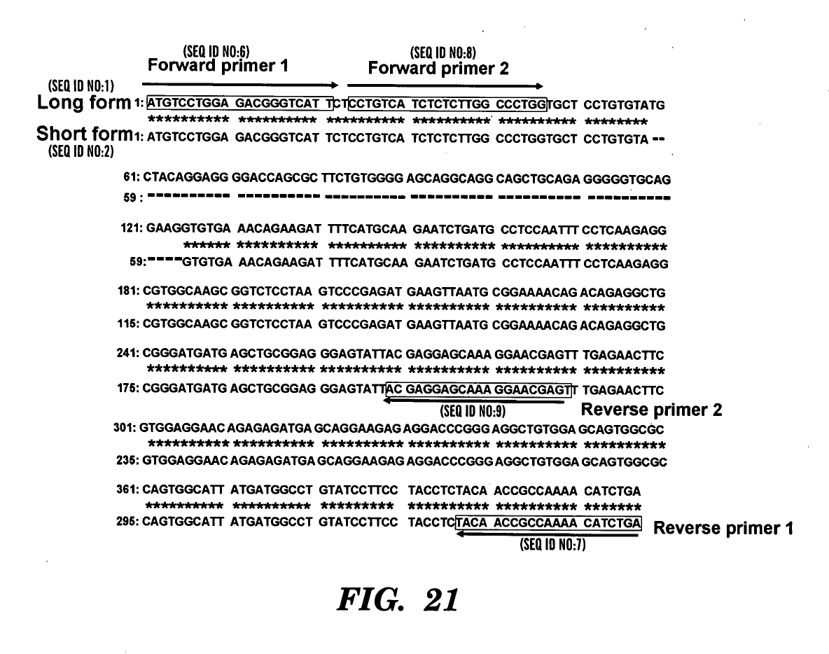

Quantities RT-PCR was performed to analyze the expression profile of MSP2, which is located on Chromosome 2 (FIG. 17) and exists as two alternatively spliced isoforms; a long isoform (SEQ ID NO:1) and a short isoform (SEQ ID NO:2) (FIG. 16), and analysis of the amino acid sequences predicts MSP3 has a signal sequence and high homology between rodent and human isoforms; the sequence identity between mouse (SEQ ID NO: 3) and rat (SEQ ID NO:4) is 94% and the sequence identity between mouse and human (SEQ ID NO:5) is 79% (FIG. 18). Analysis of total MSP3 expression using primers designed to detect both the long and short isoforms (SEQ ID NOS: 10 and 11, FIG. 19), shows MSP3 has a restricted expression in heart, brain, lung, thymus, lymph node, eye and skeletal muscle (FIG. 20A). In addition, MSP3 was expressed in C2C12 cells, a myocyte cell line, and was expression.was further induced by transfection of C2C12 cells with adenovirus expressing constitutively active Akt (MyrAkt) (FIG. 20A). Analysis of the expression of long and short isforms of MSP3 was done using isoform-specific primers to each of the long and short isoforms (SEQ ID NOS: 6-9, FIG. 21), shows the long form of MSP3 is predominantly expressed (upper band) comparatively to the short form (lower band) (FIG. 20B).

In summary, the inventors have discovered that MSP3 has a dual function as a metabolic regulator that is not shared by FGF2, as MSP3 functions as an angiogenesis factor (FIGS. 13 and 14), and also regulates sensitivity to glucose in obese mouse model (FIG. 15), whereas FGF21 only functions to regulate sensitivity to glucose.

### Example 6

### MPS5 as a Muscle Hypertrophy Factor (Myogenic Factor)

MSP5 was discovered to be differentially regulated in response to expression of muscle related transgenes and muscle growth. MSP5 corresponds to 1600015H20Rik (GenBank Accession No. AK005465) (SEQ ID NO:12)

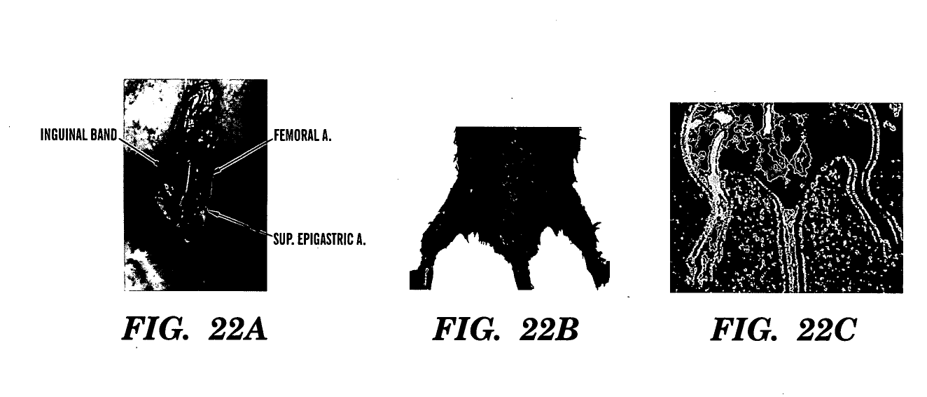

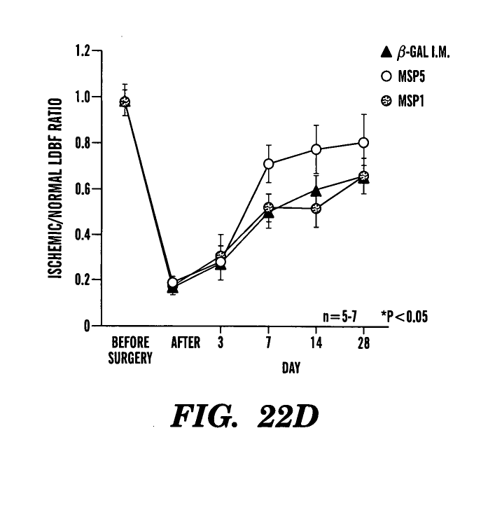

To evaluate the potential angiogenic properties of MSP5, mice at the ages of 10 weeks were subjected to unilateral hind limb surgery (J. Biol. Chem. 2004; 279:28670-28674; Circ. Res. 2005; 96(8):838-846; Circ. Res. 2006; 98(2):254-61). Adenovirus-mediated gene transfer was performed with adenoviral vectors expressing MSP5 and MSP1 by direct injection into five different sites of adductor muscle in the ischemic limb 3 days before surgery. Blood vessel growth was monitored by Laser Doppler analysis on legs and feet immediately before surgery and on postoperative days 0, 3, 7, 14, and 28 as in previₒus experiments, and Adv-MSP5 transfected mice had improved ischemic/Normal LDBF ratio as compared β-gal control or Adv-MSP1 transfected mice (FIG. 22), indicating MSP5, but not MSP1 functions to increase angiogenesis.

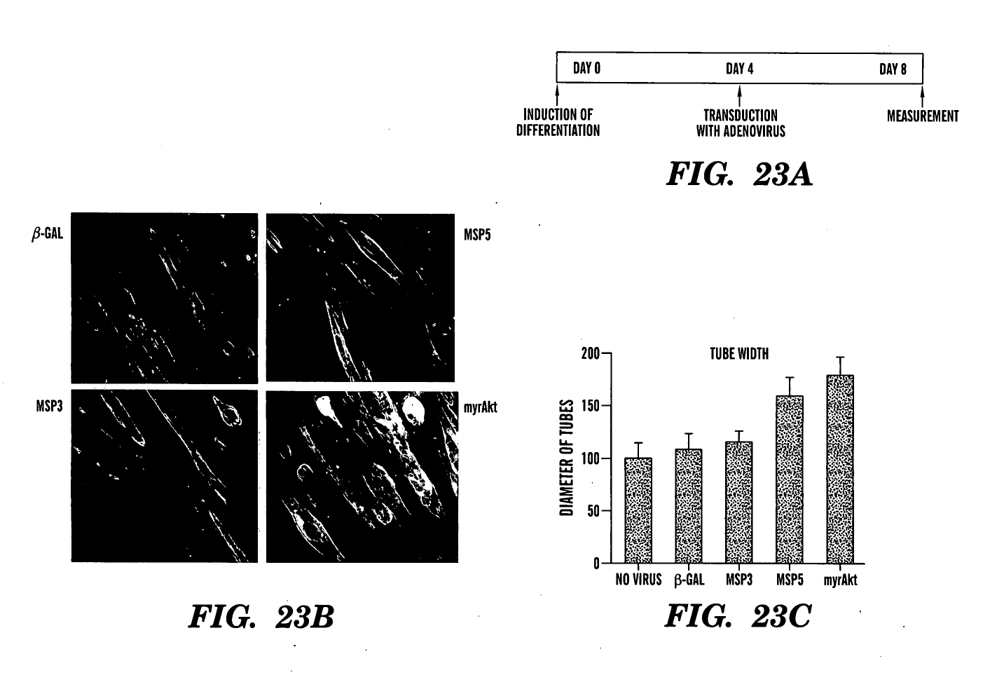

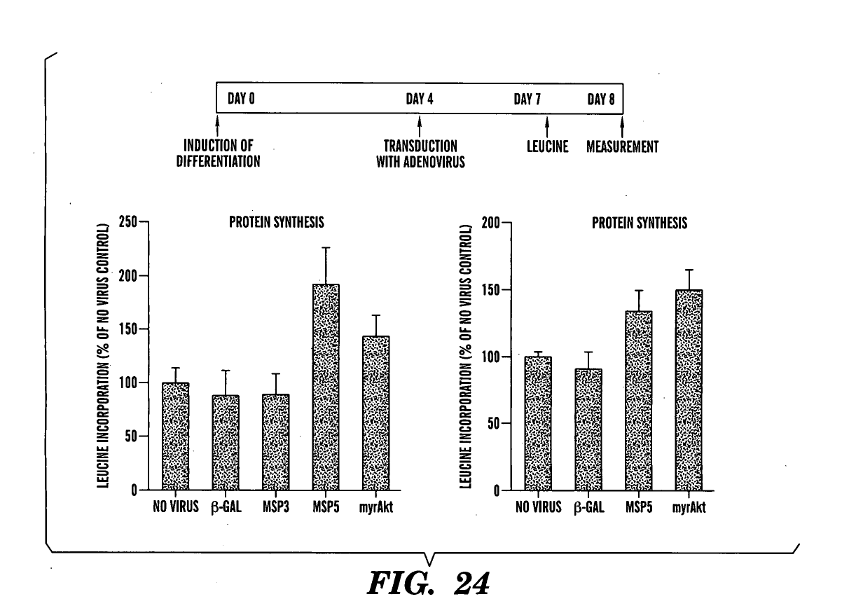

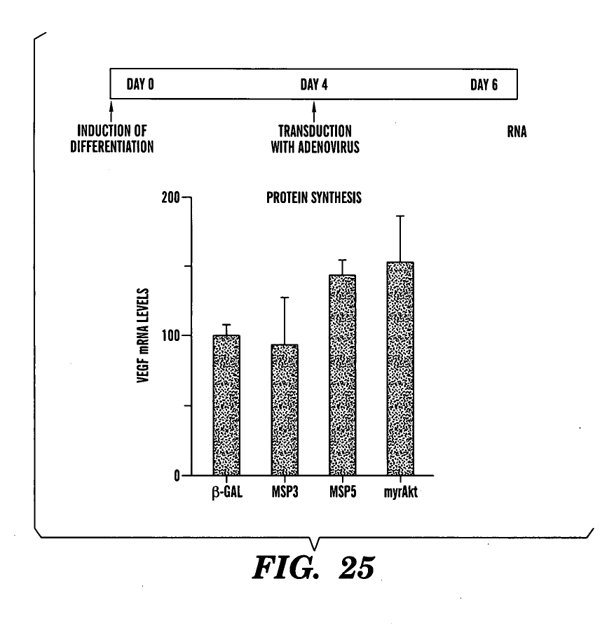

To evaluate the effect of MSP5 on growth of skeletal muscle cells in vitro, C2C12 cells four days after differentiation into myocytes were transfected with either Adv-MSP5, Adv-MSP3, adv-βGal or Adv-myrAkt and their morphology and tube diameter assessed. C2C12 cells transfected with Adv-MSP5 or adv-myrAkt are enlarged (FIG. 23B) and have increased tube diameter (FIG. 23C) compared to Adv-MSP3 or control Adv-β-gal transfected C2C12 cells. Furthermore, by assessing ³H-leucine incorporation as a measure of protein synthesis and myofibril growth, protein synthesis is increased in Adv-MSP5 and Adv-myrAkt transfected C2C12 cells compared to Adv-MSP3 and Adv-β-gal transfected (FIG. 24). C2C12 cells transfected with Adv-MSP5 or Adv-myrAkt promoted VEGF expression, an angiogenic factor, which was not observed in Adv-MSP3 or Adv-β-gal transfected cells (FIG. 25).

In summary, MSP5 functions as a muscle hypertophy factor or a myogenic factor, and promotes hypertrophy in skeletal muscle. MSP5 also stimulates angiogenesis in ischemic limb, and increases expression of VEGF in C2C12 cells. The revascularization of Adv-MSP5 transduced ischemic limb may be the consequence of increased muscle growth and increased secretion of VEGF.

### Example 7

### Insulin-like 6 (Insl6) Promotes Muscle Regeneration

Insulin-like 6 was also identified to be differentially expressed in response to expression of muscle related transgenes and muscle growth. Insulin-like 6 (Insl6) belongs to the relaxin family, and corresponds to GenBank Accession No. NM 007179 (SEQ ID NO:20)

An increase in satellite proliferation was observed 2, 4, 6, 8 and 10 weeks (w) after activation of Akt1 in the skeletal muscle of transgenic mice that have inducible expression of a muscle-related protein, for example in mice expressing constitutively active, which was not observed in control mice (FIG. 11), as determined by a centralized nuclei in muscle cells which indicate progenitor cell recruitment as myofibrils grow. Satellite cell proliferation at 2 weeks after transgene activation is shown immunohistochemistry of BrdU incorporation into DNA which was evident in muscle histological sections from mice with induced Akt but not in control mice (cont).The cells incorporating the BrdU are multinucleated and are located around the myocyte indicating the myofibril is recruiting the satellite cells (data not shown). Further analysis by immunostaining for an activated satellite marker (myo-D), double (homozygous) Akt transgenice mice show increased myoD positive satellite cells, which are not detected in muscle from control mice (data not shown). Approximately 2-4 MyoD-positive satellite cells were seen per cross-section of gastrocnemial muscle in MyoMice, but no MyoD cells were detected in the muscle from the control mice (data not shown).

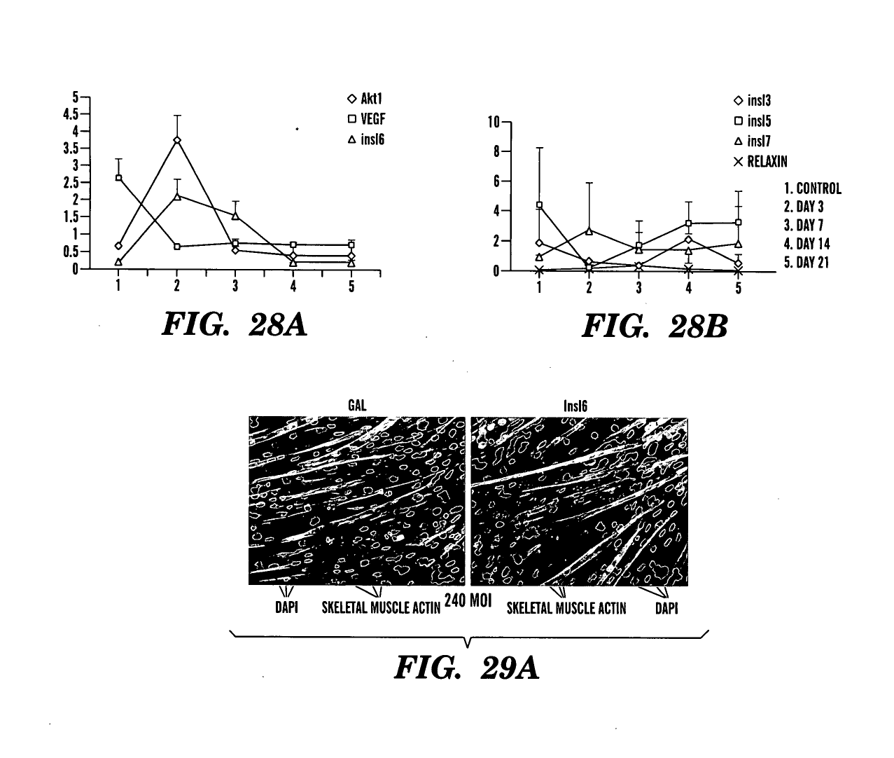

Using either transgenic mice with inducible expression of Akt, or C2C12 cells transfected with adenovirus expressing Adv-Akt, Insl6 is significantly upregulated, approximately 9-fold, in mice after induction of Akt and also upregulated in C2C12 cells expressing Adv-Akt (FIG. 26), indicating insulin-like 6 is regulated by Akt in muscle both in vitro and in vivo. Interestingly, other relaxin family members, including Insl3, Insl5, relaxin and insl7 are not regulated by Akt (FIG. 27). Furthermore, Insulin-like 6 transcript is dramatically upregulated 24-fold in transgenic mice 2 weeks after induction of Akt expression and 10-fold in C2C12 cells following transduction with Adeno-myrAkt1 (FIG. 26). In a separate experiment, Insl6 expression was analyzed in a model of muscle regeneration, where administration of cardiotoxin to tibialis anterius (TA) muscle stimulated muscle regeneration and repair. Both Akt and Insl6 transcript are upregulated during muscle regeneration following cardiotoxin administration to tibialis anterius (TA) muscle, whereas VEGF is downregulated and other members of the relaxin family (as illustrated in FIG. 41) such as Insl3, Insl5, relaxin, Insl7(relaxin 3) are not regulated in cardiotoxin-injured mouse muscle (FIG. 28).

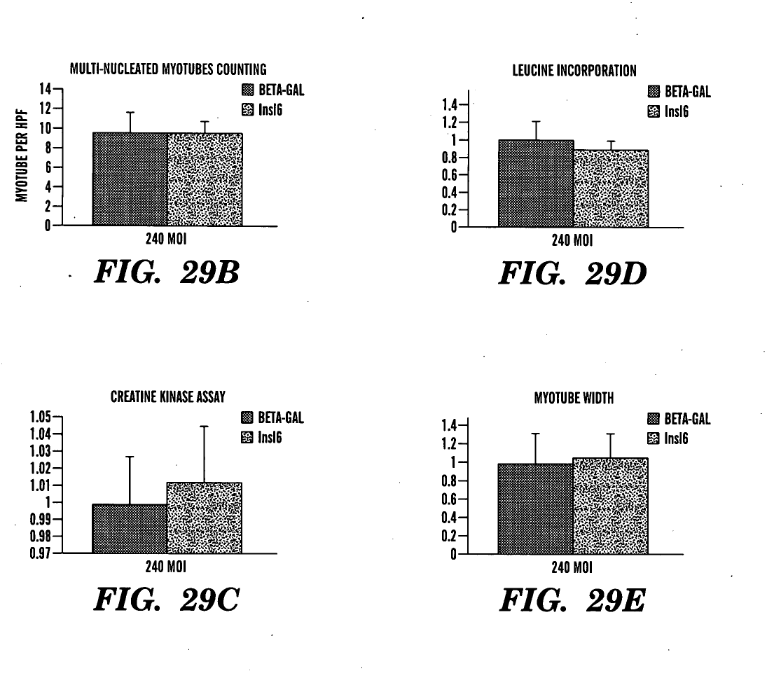

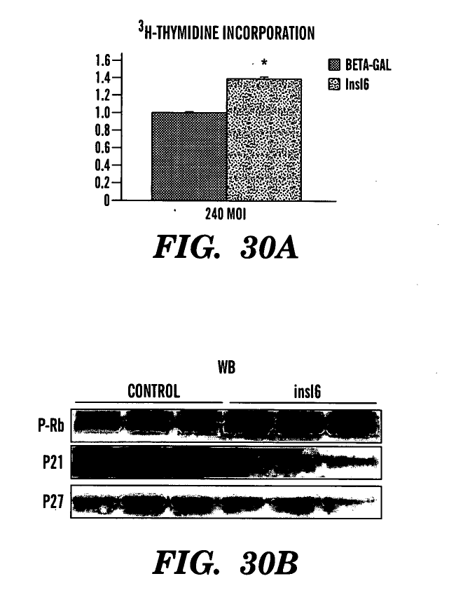

To investigate the functional role of Insl6, the inventors generated adenovirus expressing Insl6 and assessed its effect on C2C12 cells in vitro. FIG. 36 shows that C2C12 cells transfected with Adv-Insl6 or Adv-βGal at 240 MOI (multiplicities of infection), no change in morphology such as myofibril hypertrophy or differentiation of C2C12 cells occurred (FIGS. 29A and 29E), nor was there an increase in number of myotybules or change in creatine kinase expression or Leucine incorporation (FIGS 29B-E) compared to Adv-β-gal transfected C2C12 cells. Interestingly, these results with Insl6 are in contrast to what was observed for MSPS (in Example 6 above). In additional experiments, Adv-Insl6 stimulated the proliferation of satellite cells in skeletal muscle cells, as shown by increased thymidine (³H-thymidine) incorporation in skeletal muscle (FIG. 30A), which was accompanied by increase in retinoblastoma (Rb) protein and phosphorylated Rb (p-Rb) (FIG. 30B).

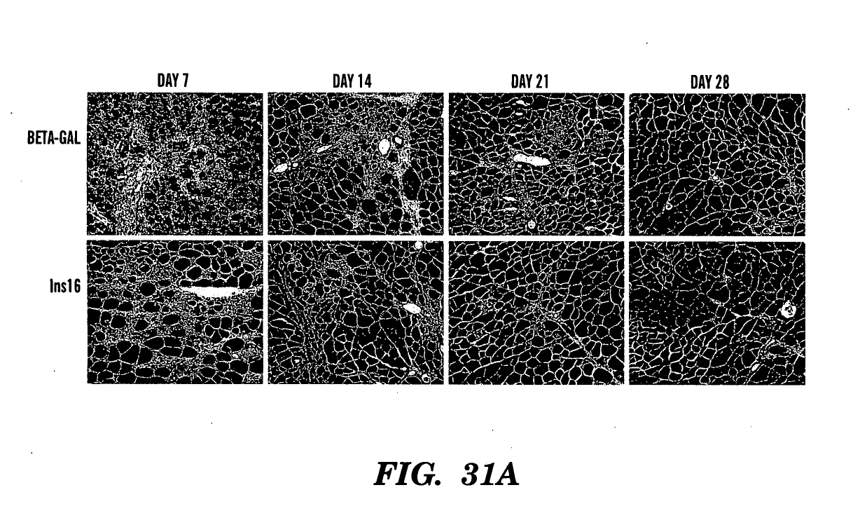

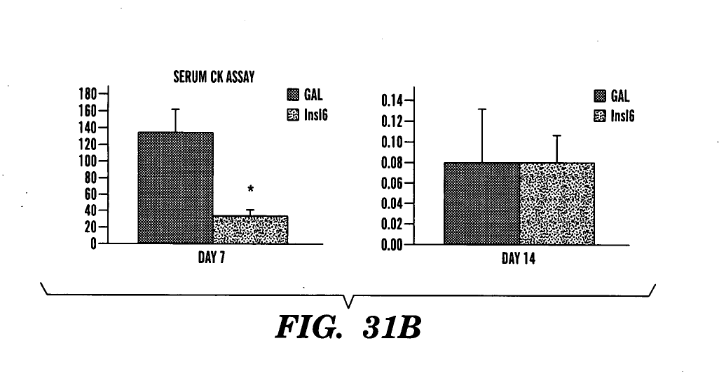

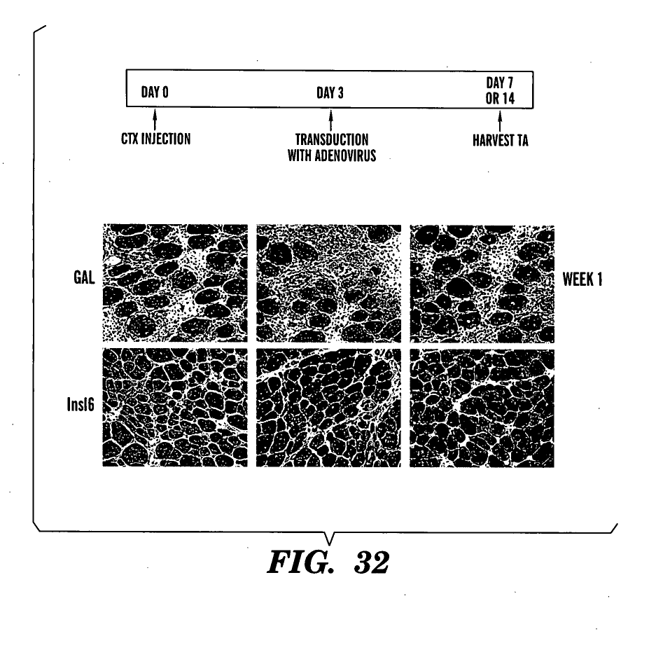

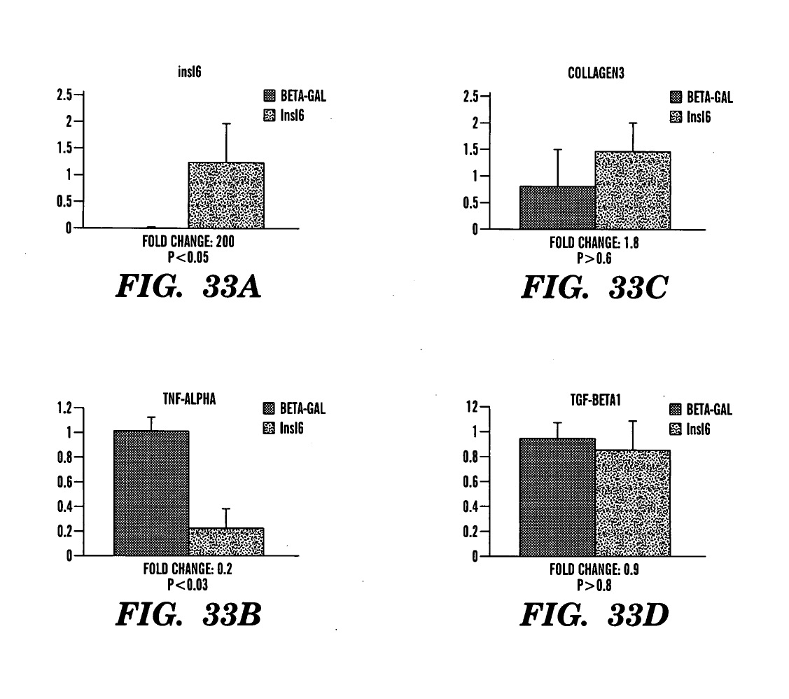

In the in vivo model of muscle regeneration using intramuscular injection of cardiotoxin (CTX) Adv-Insl6 facilitates TA muscle regeneration after cardiotoxin (CTX) injury (FIGS. 31 and 32) compared to Adv-βGal control injected mice. Improved regeneration is most notable at 7 and 14 days in histological sections (also shown in FIG. 32). At 7 days Insl6 overexpression repressed creatine kinase release into sera (lower left panel) which was not observed at 14 days (lower right panel). Quantitative analysis of transcript levels of cardiotoxin mediated muscle degeneration indicates that Insl6 mediates changes in some transcript levels, as Adv-Insl6 transfected muscle, Insl6 is increased 200-fold (p<0.05), and Adv-Insl6 expression reduces TNFα (0.2 fold, p<0.03) and TNFβ1 (0.9 fold, p>0.8), and increases the expression of collagen 3 (1.8 fold, p>0.6) (FIG. 33).

TABLE 3
Summary of Sequence listings and corresponding SEQ ID NOS:

| | | | SEQUENCE (gene |
|---|---|---|---|
| SEQ ID NO: | Clone | GenRef ID | name) |
| SEQ ID NO: 1 | MSP3 - long form (nucleotide) | NM_026754 | 1110017I16Rik |
| SEQ ID NO: 2 | MSP3 - short form (nucleotide) | NM_026754 | 1110017I16Rik |
| SEQ ID NO: 3 | MSP3 aa - mouse | NP_081030 | 1110017I16Rik |
| SEQ ID NO: 4 | MSP3 aa - rat | XP_001066258 | 1110017I16Rik |
| SEQ ID NO: 5 | MSP3 aa - human | NP_660357 | 1110017I16Rik |
| SEQ ID NO: 6 | MSP3 forward primer 1 | N/A | N/A |
| SEQ ID NO: 7 | MSP3 reverse primer 1 | N/A | N/A |
| SEQ ID NO: 8 | MSP3 forward primer 2 | N/A | N/A |
| SEQ ID NO: 9 | MSP3 reverse primer 1 | N/A | N/A |
| SEQ ID NO: 10 | MSP 3 forward primer 3 (detect both | N/A | N/A |
| | short and long forms) | | |
| SEQ ID NO: 11 | MSP 3 reverse primer 3 (detect both | N/A | N/A |
| | short and long forms) | | |
| SEQ ID NO: 12 | MSP 5 (clone 5) - nucleotide | AK005465/NM_024237 | 1600015H20Rik |
| SEQ ID NO: 13 | MSP 5 (clone 5) - amino acid - mouse | XP_001081124 | 1600015H20Rik |
| | | (Mouse) | |
| SEQ ID NO: 14 | MSP 5 (clone 2) - amino acid - rat | NP_077199 (Rat) | 1600015H20Rik |
| SEQ ID NO: 15 | MSP 5 (clone 2) - amino acid - human | NP_694946 | 1600015H20Rik |
| SEQ ID NO: 16 | MSP1 (clone 9) | BC52844 | 2160028F08Rik |
| SEQ ID NO: 17 | MSP2 (clone 8) | AK009779 | 2310043I08Rik |
| SEQ ID NO: 18 | MSP4 (clone 3) | BC025830/AK028883 | 4732466D17Rik |
| SEQ ID NO: 19 | MSP6 (FGF21) | NM-019113 | FGF21 |
| SEQ ID NO: 20 | Insl6 (nucleotide) | NM_007179/ | INSL6 |
| | | AF_156094 | |
| SEQ ID NO: 21 | insl6 (amino acid) | NP_009110 | INSL6 |
| SEQ ID NO: 22 | Akt1 (nucleic acid) | NM_005163/NM_001014431/ | RAC, PKB, PRKBA, |
| | | NM_0014432 | AKT, AKT1 |
| SEQ ID NO: 23 | Akt1 (amimo acid) | NP_001014431/NP_001014432/ | RAC, PKB, PRKBA, |
| | | NP_005154 | AKT, AKT1 |
| SEQ ID NO: 24 | Akt2 (nucleic acid) - human | NM_001626 | Akt2 |
| SEQ ID NO: 25 | Akt3 (nucleic acid) - human | NM_181690 | PKBG, RAC- |
| | | | gamma, PRKBG, |
| | | | Akt3 |
| SEQ ID NO: 26 | PI-3K | NM_002645 | P13K-C2 Alpha |
| SEQ ID NO: 27 | myostatin | NM_005259 | Myostatin, GDF8, |
| | | | MSTN |
| SEQ ID NO: 28 | Akt2 (nucleic acid) - mouse | NM_007434 | Akt2 |
| SEQ ID NO: 29 | Akt3 (nucleic acid) - mouse | NM_011785 | PKBG, RAC- |
| | | | gamma, PRKBG, |
| | | | Akt3 |

### REFERENCES

All references described herein are incorporated herein by reference.
1. Lecker S H, Solomon V, Mitch W E, Goldberg A L. Muscle protein breakdown and the critical role of the ubiquitin-proteasome pathway in normal and disease states. J. Nutr. 1999; 129:2275-2375.
2. Franzini-Armstrong C, Fischman D A. Morphogenesis of skeletal muscle fibers. In: Engel A G, Franzini-Armstrong C, editors. Myology: Basic and Clinical, 2nd ed. New York, NY: McGraw-Hill, Inc.; 1994. P. 74-96.
3. Degens H, Turek Z, Hoofd L J C, Van't Hof M A, Binkhorst R A. The relationship between capillarisation and fibre types during compensatory hypertrophy of the plantaris muscle in the rat. J. Anat. 1992; 180:455-463.
4. Degens H, Veerkamp J H, van Moerkerk H T B, Turek Z, Hoofd L J C, Binkhorst R A. Metabolic capacity, fibre type area and capillarization of rat plantaris muscle. Effects of age, overload and training and relationship with fatigue resistance. Int. J. Biochem. 1993; 25:1141-1148.
5. Ingjer F. Capillary supply and mitochondrial content of different skeletal muscle fiber types in untrained and endurance-trained men. A histochemical and ultrastructural study. Eur. J. Appl. Physiol. Occup. Physiol. 1979; 40:197-209.
6. Kano Y, Shimegi S, Masuda K, Ohmori H, Katsuta S. Morphological adaptation of capillary network in compensatory hypertrophied rat plantaris muscle. Eur. J. Appl. Physiol. Occup. Physiol. 1997; 75:97-101.
7. Carpenter S, Karpati G. Necrosis of capillaries in denervation atrophy of human skeletal muscle. Muscle Nerve 1982; 5:250-254.
8. Hudlicka O. The response of muscle to enhanced and reduced activity. In: Harris J B, Turnbull D M, editors. Bailliere's Clinical Endocrinology and Metabolism. Toronto: Bailliere-Tindal; 1990. p. 417-439.
9. Plyley M J, Olmstead B J, Noble E G. Time course of changes in capillarization in hypertrophied rat plantaris muscle. J. Appl. Physiol. 1998; 84:902-907.
10. Musaro A, McCullagh K, Paul A, Houghton L, Dobrowolny G, Molinaro M, Barton E R, Sweeney H L, Rosenthal N. Localized Igf-1 transgene expression sustains hypertrophy and regeneration in senescent skeletal muscle. Nat. Genet. 2001; 27:195-200.
11. Sutrave P, Kelly A M, Hughes S H. ski can cause selective growth of skeletal muscle in transgenic mice. Genes Dev. 1990; 4:1462-1472.
12. Lai K M, Gonzalez M, Poueymirou W T, Kline W O, Na E, Zlotchenko E, Stitt T N, Economides A N, Yancopoulos G D, Glass D J. Conditional activation of akt in adult skeletal muscle induces rapid hypertrophy. Mol. Cell. Biol. 2004; 24:9295-9304.
13. Reaven G M. Pathophysiology of insulin resistance in human disease. Physiol. Rev. 1995; 75:473-486.
14. Mueckler M. Insulin resistance and the disruption of Glut4 trafficking in skeletal muscle. J. Clin. Invest. 2001; 107(10):1211-1213.
15. Kadowaki T. Insights into insulin resistance and type 2 diabetes from knockout mouse models. J. Clin. Invest. 2000; 106:459-465.
16. Lillioja S, Mott D M, Spraul M, Ferraro R, Foley J E, Ravussin E, Knowler W C, Bennett P H, Bogardus C. Insulin resistance and insulin secretory dysfunction as precursors of non-insulin-dependent diabetes mellitus. Prospective studies of Pima Indians. N. Engl. J. Med. 1993; 329:1988-1992.
17. Martin B C, Warram J H, Krolewski A S, Bergman R N, Soeldner J S, Kahn C R. Role of glucose and insulin resistance in development of type 2 diabetes mellitus: results of a 25-year follow-up study. Lancet 1992; 340:925-929.
18. Knowler W C, Barrett-Connor E, Fowler S E, Hamman R F, Lachin J M, Walker E A, Nathan D M. Reduction in the incidence of type 2 diabetes with lifestyle intervention or metformin. N. Engl. J. Med. 2002; 346:393-403.
19. Datta S R, Brunet A, Greenberg M E. Cellular survival: a play in three Aids. Genes Dev. 1999; 13:2905-2927.
20. Chen W S, Xu P Z, Gottlob K, Chen M L, Sokol K, Shiyanova T, Roninson I, Weng W, Suzuki R, Tobe K, Kadowaki T, Hay N. Growth retardation and increased apoptosis in mice with homozygous disruption of the Akt1 gene. Genes Dev. 2001; 15:2203-2208.
21. Verdu J, Buratovich M A, Wilder E L, Birnbaum M J. Cell-autonomous regulation of cell and organ growth in Drosophila by Akt/PKB. Nat. Cell Biol. 1999; 1:500-506.
22. Ueki K, Yamamoto-Honda R, Kaburagi Y, Yamauchi T, Tobe K, Burgering B M, Coffer P S, Komuro I, Akanuma Y, Yazaki Y, Kadowaki T. Potential role of protein kinase B in insulin-induced glucose transport, glycogen synthesis, and protein synthesis. J. Biol. Chem. 1998; 273:5315-5322.
23. Rommel C, Clarke B A, Zimmermann S, Nunez L, Rossman R, Reid K, Moelling K, Yancopoulos G D, Glass D J. Differentiation stage-specific inhibition of the Raf-MEK-ERK pathway by Akt. Science 1999; 286:1738-1741.
24. Bodine S C, Stitt T N, Gonzalez M, Kline W O, Stover G L, Bauerlein R, Zlotchenko E, Scrimgeour A, Lawrence J C, Glass D J, Yancopoulos G D. Akt/mTOR pathway is a crucial regulator of skeletal muscle hypertrophy and can prevent muscle atrophy in vivo. Nat. Cell Biol. 2001; 3:1014-1019.
25. Hixon M L, Muro-Cacho C, Wagner M W, Obejero-Paz C, Millie E, Fujio Y, Kureishi Y, Hassold T, Walsh K, Gualberto A. Akt1/PKB upregulation leads to vascular smooth muscle cell hypertrophy and polyploidization. J. Clin. Invest. 2000; 106:1011-1020.
26. Ushio-Fukai M, Alexander R W, Akers M, Yin Q, Fujio Y, Walsh K, Griendling K K. Reactive oxygen species mediate the activation of Akt/protein kinase B by angiotensin II in vascular smooth muscle cells. J. Biol. Chem. 1999; 274:22699-22704.
27. Shiojima I, Sato K, Izumiya Y, Schiekofer S, Ito M, Liao R, Colucci W, Walsh K. Disruption of coordinated cardiac hypertrophy and angiogenesis contributes to the transition to heart failure. J. Clin. Invest. 2005; 115:2108-2118.
28. Takahashi A, Kureishi Y, Yang J, Luo Z, Guo K, Mukhopadhyay D, Ivashchenko Y, Branellec D, Walsh K. Myogenic Akt signaling regulates blood vessel recruitment during myofiber growth. Mol. Cell. Biol. 2002; 22:4803-4814.

1. A method for identifying factors associated with muscle growth, angiogenesis, obesity, glucose regulation, muscle regeneration, muscle hypertrophy comprising performing an assay to identify genes differentially expressed between:
(a) muscle cells containing a heterologous nucleic acid construct of a muscle-related transgene, wherein the muscle cells express the muscle-related transgene for a period of time; and
(b) muscle cells not containing the heterologous nucleic acid construct of a muscle-related transgene;
wherein the differentially expressed genes encode for factors associated with at least one of muscle growth, angiogenesis, obesity, glucose regulation, muscle regeneration, muscle hypertrophy.

2. The method of claim 1, wherein the nucleic acid construct of a muscle-related transgene comprises a nucleic acid sequence encoding a muscle related gene operatively linked to a muscle promoter, wherein the muscle related gene is a positive regulator of muscle growth.

3. The method of claim 1, wherein the nucleic acid construct of a muscle-related transgene comprises a nucleic acid sequence encoding an inhibitor to a muscle related gene operatively liked to a muscle promoter, wherein the muscle related gene is a negative regulator of muscle growth.

4. The method of claim 1, further comprising performing an assay to identify genes differentially expressed between:
(c) muscle cells comprising a nucleic acid construct of a muscle-related transgene, wherein the muscle cells express the muscle-related transgene and
(d) muscle cells comprising a nucleic acid construct of a muscle-related transgene, wherein the muscle cells express the muscle-related transgene for a period of time and
wherein the genes differentially expressed between (c) and (d) are compared to the genes differentially expressed between (a) and (b),
wherein a set of genes that is differentially expressed between (c) and (d) and are also differentially expressed between (a) and (b) code for factors associated with muscle growth.

5. The method of claim 1, wherein expression for a period of time is continual expression.

6. The method of claim 1, wherein expression for a period of time is at least one period of time where expression occurs followed by at least one period of repressed expression.

7. The method of claim 1, wherein expression for a period of time is at least one period of time of repressed expression followed by at least one period of time where expression occurs.

8. The method of claim 2, where in the muscle related gene is Akt or a homologue or variant thereof.

9. The method of claim 8, wherein Akt is a constitutively active isoform of Akt.

10. (canceled)

11. The method of claim 3, wherein the muscle-related transgene is PI-3 kinase or a homologue or variant thereof.

12. The method of claim 11, wherein the muscle-related transgene is myostatin.

13. The method of claim 11, wherein the inhibitor of a muscle-related transgene is a nucleic acid inhibitor or a dominant negative form of the muscle-related transgene.

14. The method of claim 13, wherein the nucleic acid inhibitor is selected from the group consisting of RNAi, siRNA, shRNAi, miRNA, antisense nucleic acids, antisense oligonucleic acid (ASO), neutralizing antibodies and variants thereof.

15. (canceled)

16. (canceled)

17. The method of claim 2 or 3, wherein the skeletal muscle promoter is selected from a group of MCK, α-myosin heavy chain, myosin-light chain 2, SM22a, or combinations or homologues or variants thereof.

18. The method of claim 2 or 3, wherein the muscle promoter is an inducible muscle promoter.

19. The method of claim 18, wherein the inducible muscle promoter is selected from the group of TetR, FK506/VP16/p65/castradiol, PU486/mitepristone, diphenylmuristoerone, rapamycin, Cre/LoxP and combinations thereof.

20. (canceled)

21. (canceled)

22. (canceled)

23. (canceled)

24. (canceled)

25. (canceled)

26. (canceled)

27. The method of claim 1, wherein the animal is a transgenic animal.

28. (canceled)

29. A transgenic animal comprising a nucleic acid construct of a muscle-related transgene and progeny thereof, wherein the nucleic acid construct of a muscle-related transgene comprises a nucleic acid sequence encoding a muscle related gene operatively linked to a muscle promoter, wherein the muscle related gene is a positive regulator of muscle growth.

30. A transgenic animal comprising a nucleic acid construct of a muscle-related transgene and progeny thereof, wherein the nucleic acid construct of a muscle-related transgene comprises a nucleic acid sequence encoding an inhibitor to a muscle related gene operatively linked to a muscle promoter, wherein the muscle related gene is a negative regulator of muscle growth.

31.-43. (canceled)

44. A cell line derived from the transgenic animal of claim 29.

45. A cell line derived from the transgenic animal of claim 30.

46. The cell line of claims 44 or 45, wherein the cell line is a skeletal muscle cell line.
